(19) United States
(12) Patent Application Publication
(10) Pub. No.: US 2026/0131052 A1
(43) Pub. Date: May 14, 2026

(54) ENGINEERED TISSUE CONSTRUCTS FOR THE TREATMENT OF HYPERAMMONEMIA

(71) Applicant: Satellite Biosciences, Inc., Cambridge, MA (US)

(72) Inventors: Arnav CHHABRA, Mansfield, MA (US); Amanda CHEN, Cambridge, MA (US); Joseph E. MARTURANO, Bedford, MA (US); Keval N. VYAS, Somerville, MA (US); Thomas Jay LOWERY, JR., Sudbury, MA (US); Fabiola MUNARIN, Smithfield, RI (US)

(21) Appl. No.: 18/704,217

(22) PCT Filed: Oct. 25, 2022
(86) PCT No.: PCT/US2022/047745

§ 371 Date: Apr. 24, 2024

Related U.S. Application Data
(60) Provisional application No. 63/271,552, filed on Oct. 25, 2021.

(51) Int. Cl. A61L 27/38; A61L 27/22; A61L 27/52

(52) U.S. Cl. CPC A61L 27/3834; A61L 27/225; A61L 27/52

(57) ABSTRACT

The present disclosure provides implantable engineered tissue constructs having hepatocytes and stromal cells for the treatment of hyperammonemia.

### FIELD OF THE INVENTION

The present disclosure concerns the use of engineered tissue constructs include hepatocytes and stromal cells for treating hyperammonemia.

### BACKGROUND OF THE INVENTION

Ammonia is highly toxic and generated during metabolism in all organs. Hyperammonemia is caused by the decreased detoxification and/or increased production of ammonia. In mammals, the urea cycle detoxifies ammonia by enzymatically converting ammonia into urea, which is then removed in the urine. For example, decreased ammonia detoxification may be caused by a urea cycle disorder (UCD) in which urea cycle enzymes are defective, such as a deficiency in ornithine transcarbamylase. The National Urea Cycle Disorders Foundation estimates that the prevalence of UCDs is 1 in 8,500 births. In addition, non-UCD disorders, such as organic acidemia, congenital lactic acidosis, fatty acid oxidation defect, dibasic amino acid deficiencies, transient hyperammonemia, Reye syndrome, severe perinatal asphyxia, virus-associated hyperammonemia, infection, drug-induced hyperammonemia, liver disease, acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder, gastric bypass surgery, gastrointestinal bleeding, malnutrition, generalized seizures, organ transplantation, and renal failure, can also cause hyperammonemia. Hyperammonemia can produce neurological manifestations, e.g., seizures, ataxia, stroke-like lesions, coma, psychosis, vision loss, acute encephalopathy, cerebral edema, as well as vomiting, respiratory alkalosis, hypothermia, or death.

Current therapies for hyperammonemia aim to reduce ammonia excess but are widely regarded as suboptimal. For example, in UCD, most patients require substantially modified diets consisting of protein restriction. However, a low-protein diet must be carefully monitored because when protein intake is too restrictive, the body breaks down muscle and consequently produces ammonia. Therefore, many patients require supplementation with ammonia scavenging drugs, such as sodium phenylbutyrate, sodium benzoate, and glycerol phenylbutyrate, and one or more of these drugs must be administered three to four times per day. Side effects of these drugs include nausea, vomiting, irritability, anorexia, and menstrual disturbance in females. In children, the delivery of food and medication may require a gastrostomy tube surgically implanted in the stomach or a nasogastric tube manually inserted through the nose into the stomach. When these treatment options fail, a liver transplant may be required. Thus, there is significant unmet need for effective, reliable, and long-term treatment for disorders associated with hyperammonemia.

### SUMMARY OF THE INVENTION

The present disclosure provides compositions and methods that can be used for treating hyperammonemia. Using the compositions and methods of the disclosure, a patient (e.g., a mammalian patient, such as a human patient) having a urea cycle disorder, organic acidemia, congenital lactic acidosis, fatty acid oxidation defect, dibasic amino acid deficiencies, transient hyperammonemia, Reye syndrome, severe perinatal asphyxia, virus-associated hyperammonemia, infection, drug-induced hyperammonemia, liver disease, acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder, gastric bypass surgery, gastrointestinal bleeding, malnutrition, generalized seizures, organ transplantation, or renal failure may be administered an engineered tissue construct including a population of hepatocytes and a population of fibroblasts in amounts effective to treat hyperammonemia.

In one aspect, the disclosure provides a method of treating hyperammonemia in a subject having a urea cycle disorder, organic acidemia, congenital lactic acidosis, fatty acid oxidation defect, dibasic amino acid deficiencies, transient hyperammonemia, Reye syndrome, severe perinatal asphyxia, virus-associated hyperammonemia, infection (e.g., a urinary tract infection or an infection caused by Proteus mirabilis, E. coli, or Klebsiella), drug-induced hyperammonemia (e.g., caused by the drug valproate acid, topiramate, carbamazepine, salicylate, sulfadiazine, a carbonic anhydrase inhibitor, a carbonic anhydrase inhibitor with valproate acid, or a chemotherapy), liver disease (e.g., biliary atresia, alpha-1 antitrypsin deficiency, Wilson disease, cystic fibrosis, galactosemia, or tyrosinemia), acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder (e.g., multiple myeloma or acute leukemia), gastric bypass surgery, gastrointestinal bleeding, malnutrition, generalized seizures, organ transplantation, or renal failure, the method including implanting one or more engineered tissue construct including a population of hepatocytes and an optional population of stromal cells (e.g., fibroblasts) in amounts effective to treat hyperammonemia in the subject.

In another aspect, the disclosure provides a method of reducing ammonia levels in a subject in need thereof (e.g., a subject having urea cycle disorder, organic acidemia, congenital lactic acidosis, fatty acid oxidation defect, dibasic amino acid deficiencies, transient hyperammonemia, Reye syndrome, severe perinatal asphyxia, virus-associated hyperammonemia, infection (e.g., a urinary tract infection or an infection caused by Proteus mirabilis, E. coli, or Klebsiella), drug-induced hyperammonemia (e.g., caused by the drug valproate acid, topiramate, carbamazepine, salicylate, sulfadiazine, a carbonic anhydrase inhibitor, a carbonic anhydrase inhibitor with valproate acid, or a chemotherapy), liver disease (e.g., biliary atresia, alpha-1 antitrypsin deficiency, Wilson disease, cystic fibrosis, galactosemia, or tyrosinemia), acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder (e.g., multiple myeloma or acute leukemia), gastric bypass surgery, gastrointestinal bleeding, malnutrition, generalized seizures, organ transplantation, or renal failure), the method includes implanting one or more engineered tissue construct including a population of hepatocytes and an optional population of stromal cells (e.g., fibroblasts) in amounts effective to reduce ammonia levels in the subject.

In some embodiments of any of the foregoing aspects, the population of hepatocytes includes an amount of hepatocytes that is equivalent to 0.5% to 30% (e.g., 0.5% to 30%, 0.6% to 30%, 0.7% to 30%, 0.8% to 30%, 0.9% to 30%, 1% to 30%, 2% to 30%, 3% to 30%, 4% to 30%, 5% to 30%, 10% to 30%, or 20% to 30%) of the total liver mass of the subject.

In some embodiments of any of the foregoing aspects, the population of hepatocytes includes an amount of hepatocytes that is equivalent to 0.5% to 20% (e.g., 0.5% to 20%, 0.6% to 20%, 0.7% to 20%, 0.8% to 20%, 0.9% to 20%, 1% to 20%, 2% to 20%, 3% to 20%, 4% to 20%, 5% to 20%, or 10% to 20%) of the mass of the liver reserve of the subject.

In some embodiments of any of the foregoing aspects, the population of hepatocytes includes 3×10⁵ to 1.8×10¹¹ (e.g., from about 4×10⁵ to about 1.8×10¹¹, from about 5×10⁵ to about 1.8×10¹¹, from about 6×10⁵ to about 1.8×10¹¹, from about 7×10⁵ to about 1.8×10¹¹, from about 8×10⁵ to about 1.8×10¹¹, from about 9×10⁵ to about 1.8×10¹¹, from about 1×10⁶ to about 1.8×10¹¹, from about 2×10⁶ to about 1.8×10¹¹, from about 3×10⁶ to about 1.8×10¹¹, from about 4×10⁶ to about 1.8×10¹¹, from about 5×10⁶ to about 1.8×10¹¹, from about 6×10⁶ to about 1.8×10¹¹, from about 7×10⁶ to about 1.8×10¹¹, from about 8×10⁶ to about 1.8×10¹¹, from about 9×10⁶ to about 1.8×10¹¹, from about 1×10⁷ to about 1.8×10¹¹, from about 2×10⁷ to about 1.8×10¹¹, from about 3×10⁷ to about 1.8×10¹¹, from about 4×10⁷ to about 1.8×10¹¹, from about 5×10⁷ to about 1.8×10¹¹, from about 6×10⁷ to about 1.8×10¹¹, from about 7×10⁷ to about 1.8×10¹¹, from about 8×10⁷ to about 1.8×10¹¹, from about 9×10⁷ to about 1.8×10¹¹, from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8×10¹¹, from about 7×10⁸ to about 1.8×10¹¹, from about 8×10⁸ to about 10.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×10⁹ to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes.

In some embodiments of any of the foregoing aspects, the optional population of stromal cells (e.g., fibroblasts) includes up to 1.8×10¹² (e.g., from about 1 to about 1.8×10¹², from about 10 to about 1.8×10¹², from about 100 to about 1.8×10¹², from about 1×10³ to about 1.8×10¹², from about 2×10³ to about 1.8×10¹², from about 3×10³ to about 1.8×10¹², from about 4×10³ to about 1.8×10¹², from about 5×10³ to about 1.8×10¹², from about 6×10³ to about 1.8×10¹², from about 7×10³ to about 1.8×10¹², from about 8×10³ to about 1.8×10¹², from about 9×10³ to about 1.8×10¹², from about 1×10⁴ to about 1.8×10¹², from about 2×10⁴ to about 1.8×10¹², from about 3×10⁴ to about 1.8×10¹², from about 4×10⁴ to about 1.8×10¹², from about 5×10⁴ to about 1.8×10¹², from about 6×10⁴ to about 1.8×10¹², from about 7×10⁴ to about 1.8×10¹², from about 8×10⁴ to about 1.8×10¹², from about 9×10⁴ to about 1.8×10¹², from about 1×10⁵ to about 1.8×10¹², from about 2×10⁵ to about 1.8×10¹², from about 3×10⁵ to about 1.8×10¹², from about 4×10⁵ to about 1.8×10¹², from about 5×10⁵ to about 1.8×10¹², from about 6×10⁵ to about 1.8×10¹², from about 7×10⁵ to about 1.8×10¹², from about 8×10⁵ to about 1.8×10¹², from about 9×10⁵ to about 1.8×10¹², from about 1×10⁶ to about 1.8×10¹², from about 2×10⁶ to about 1.8×10¹², from about 3×10⁶ to about 1.8×10¹², from about 4×10⁶ to about 1.8×10¹², from about 5×10⁶ to about 1.8×10¹², from about 6×10⁶ to about 1.8×10¹², from about 7×10⁶ to about 1.8×10¹², from about 8×10⁶ to about 1.8×10¹², from about 9×10⁶ to about 1.8×10¹², from about 1×10⁷ to about 1.8×10¹², from about 2×10⁷ to about 1.8×10¹², from about 3×10⁷ to about 1.8×10¹², from about 4×10⁷ to about 1.8×10¹², from about 5×10⁷ to about 1.8×10¹², from about 6×10⁷ to about 1.8×10¹², from about 7×10⁷ to about 1.8×10¹², from about 8×10⁷ to about 1.8×10¹², from about 9×10⁷ to about 1.8×10¹², from about 1×10⁸ to about 1.8×10¹², from about 2×10⁸ to about 1.8×10¹², from about 3×10⁸ to about 1.8×10¹², from about 4×10⁸ to about 1.8×10¹², from about 5×10⁸ to about 1.8×10¹², from about 6×10⁸ to about 1.8×10¹², from about 7×10⁸ to about 1.8×10¹², from about 8×10⁸ to about 1.8×10¹², from about 9×10⁸ to about 1.8×10¹², from about 1×10⁹ to about 1.8×10¹², from about 2×10⁹ to about 1.8×10¹², from about 3×10⁹ to about 1.8×10¹², from about 4×10⁹ to about 1.8×10¹², from about 5×10⁹ to about 1.8×10¹², from about 6×10⁹ to about 1.8×10¹², from about 7×10⁹ to about 1.8×10¹², from about 8×10⁹ to about 1.8×10¹², from about 9×10⁹ to about 1.8×10¹², from about 1×10¹⁰ to about 1.8×10¹², from about 2×10¹⁰ to about 1.8×10¹², from about 3×10¹⁰ to about 1.8×10¹², from about 4×10¹⁰ to about 1.8×10¹², from about 5×10¹⁰ to about 1.8×10¹², from about 6×10¹⁰ to about 1.8×10¹², from about 7×10¹⁰ to about 1.8×10¹², from about 8×10¹⁰ to about 1.8×10¹², from about 9×10¹⁰ to about 1.8×10¹², from about 1×10¹¹ to about 1.8×10¹², from about 2×10¹¹ to about 1.8×10¹², from about 3×10¹¹ to about 1.8×10¹², from about 4×10¹¹ to about 1.8×10¹², from about 5×10¹¹ to about 1.8×10¹², from about 6×10¹¹ to about 1.8×10¹², from about 7×10¹¹ to about 1.8×10¹², from about 8×10¹¹ to about 1.8×10¹², from about 9×10¹¹ to about 1.8×10¹², or from about 1×10¹² to about 1.8×10¹²) stromal cells (e.g., fibroblasts).

In some embodiments, the hepatocytes are primary human hepatocytes. In some embodiments, the hepatocytes are derived from stem cells (e.g., induced pluripotent stem cells).

In some embodiments, the stromal cells are fibroblasts. In some embodiments, the fibroblasts are selected from the group consisting of normal human dermal fibroblasts and neonatal foreskin fibroblasts. For example, in some embodiments, the fibroblasts are neonatal foreskin fibroblasts. In some embodiments, the fibroblasts are normal human dermal fibroblasts.

In some embodiments of any of the foregoing aspects, the ratio of hepatocytes to stromal cells (e.g., fibroblasts) is between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

In some embodiments of any of the foregoing aspects, the implant is from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume.

In some embodiments, the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). In some embodiments, the density of hepatocytes is 3 M/mL to 12 M/mL.

In some embodiments of any of the foregoing aspects, the engineered tissue construct further includes a biocompatible hydrogel scaffold. For example, in some embodiments, the biocompatible scaffold includes fibrin. In some embodiments, the biocompatible scaffold includes heparin. In some embodiments, the heparin is a synthetic heparin mimetic.

In some embodiments, the engineered tissue construct is implanted into the subject at an implantation site selected from the group consisting of the peritoneum (e.g., retroperitoneum), peritoneal cavity (e.g., omentum or mesentery), rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization. For example, in some embodiments, the peritoneum is the retroperitoneum. In some embodiments, the peritoneal cavity is the omentum. In some embodiments, the peritoneal cavity is the mesentery. In some embodiments, the omentum is the greater omentum or the omental bursa. In some embodiments, the omentum is the pedicled omentum. In some embodiments, the mesentery is the small intestinal mesentery. In some embodiments, the implant is implanted into the subject as a pedicled omental wrap or an omental wrap. In some embodiments, the implantation site is a site that is suitable for neovascularization.

In some embodiments, the engineered tissue construct is implanted into the subject at an implantation site that has a microvessel density of greater than about 3.6 vessels/mm² (e.g., greater than about 3.7 vessels/mm², 3.8 vessels/mm², 3.9 vessels/mm², 4 vessels/mm², 4.1 vessels/mm², 4.2 vessels/mm², 4.3 vessels/mm², 4.4 vessels/mm², 4.5 vessels/mm², 5 vessels/mm², 6 vessels/mm², 7 vessels/mm², 8 vessels/mm², 9 vessels/mm², 10 vessels/mm², 50 vessels/mm², 100 vessels/mm², 200 vessels/mm², 300 vessels/mm², 400 vessels/mm², 500 vessels/mm², 1000 vessels/mm², 2000 vessels/mm², 3000 vessels/mm², 4000 vessels/mm², or 4500 vessels/mm²).

In some embodiments, the engineered tissue construct provides a microenvironment that promotes the persistence of hepatocyte survival for at least three months (e.g., at least three months, at least four months, at least five months, at least six months, at least one year, at least five years, or at least ten years) in the subject.

In some embodiments, the subject is a human.

In some embodiments, following implantation of the engineered tissue construct, the subject exhibits a level of serum ammonia of less than or equal to about 90 μmol/L (e.g., less than about 89 μmol/L, 88 μmol/L, 87 μmol/L, 86 μmol/L, 85 μmol/L, 84 μmol/L, 83 μmol/L, 82 μmol/L, 81 μmol/L, 80 μmol/L, 79 μmol/L, 78 μmol/L, 77 μmol/L, 76 μmol/L, 75 μmol/L, 74 μmol/L, 73 μmol/L, 72 μmol/L, 71 μmol/L, 70 μmol/L, 69 μmol/L, 68 μmol/L, 67 μmol/L, 66 μmol/L, 65 μmol/L, 64 μmol/L, 63 μmol/L, 62 μmol/L, 61 μmol/L, 60 μmol/L, 50 μmol/L, 40 μmol/L, 30 μmol/L, 20 μmol/L, 25 μmol/L, or 10 μmol/L).

In some embodiments, following implantation of the engineered tissue construct, the subject exhibits a change in one or more parameters in a blood test relative to a reference level. In some embodiments, the blood test is a liver function test. For example, in some embodiments, the one or more parameters includes the level of gamma-glutamyl transferase, alkaline phosphatase, aspartate aminotransferase, alanine aminotransferase, or albumin.

In some embodiments, following implantation of the engineered tissue construct, the subject exhibits an improvement in a test of gallbladder ejection fraction. For example, in some embodiments, the test is a hepatobiliary iminodiacetic acid scan.

In another aspect, the disclosure provides a kit including an engineered tissue construct, wherein the kit further includes a package insert instructing a user of the kit to implant the engineered tissue construct to the subject in accordance with the method of any one of the foregoing embodiments.

### BRIEF DESCRIPTION OF THE DRAWINGS

FIG. 1 is a schematic showing the experimental outline of a study to validate an ammonia challenge in mice implanted with an engineered tissue construct. Beginning on day −6, wild-type (B6EiC3SnF1/J) and transgenic B6EiC3Sn a/A-Otc^{spf-ash}/J (spf^{ash}) mice underwent immunosuppression every two days or were untreated. Groups 1 (healthy controls) and 2 (unhealthy controls) were not subjected to surgery. On day 1, a pre-implantation blood draw was taken, followed by implantation of two engineered tissue constructs per mouse for Group 3 onto the parametrial fat pad in the intraperitoneal space, each construct including 1.41×10⁶ primary human hepatocytes and 2.82×10⁶ normal human dermal fibroblasts. Following implantation, mice were challenged with 7.5 mmol/kg NH₄Cl on days 4, 11, 18, 25, and 32, which was administered intraperitoneally. On days 7, 14, 21, and 28 a urine draw was collected, followed by sacrifice on day 32.

FIG. 2 is a graph showing the serum human albumin levels in spf^{ash} mice implanted with two engineered tissue constructs, as described in FIG. 1.

FIG. 3 shows the clinical observation of wild-type Group 1 healthy no-surgery control mice (B6EiC3SnF1/J), transgenic spf^{ash} Group 2 unhealthy no-surgery control mice, and transgenic spf^{ash} Group 3 mice challenged with 7.5 mmol/kg NH₄Cl and implanted with two engineered tissue constructs, as described in FIG. 1.

FIG. 4 is a graph showing the serum ammonia levels in wild-type healthy no-surgery control mice (C57BL/6), spf^{ash} unhealthy no-surgery control mice, and spf^{ash} mice challenged with 7.5 mmol/kg NH₄Cl and implanted with two engineered tissue constructs, as described in FIG. 1.

FIG. 5 is a graph showing the serum ammonia levels in spf^{ash} unhealthy no-surgery control mice and spf^{ash} mice implanted with two engineered tissue constructs challenged with 7.5 mmol/kg NH₄Cl, as described in FIG. 1.

FIG. 6 is a schematic showing the steps of a manufacturing build for engineered tissue constructs. In Step 1, stromal cells (e.g., fibroblasts) (e.g., neonatal human dermal fibroblasts) are thawed from their respective working cell bank (WCB), expanded, and tested to measure viability and cell count. Hepatocytes (e.g., primary human hepatocytes (PHH)) are thawed from the hepatocyte master cell bank (MCB) and tested to measure viability and cell count (Step 2); prior to being combined at a ratio (e.g., 1:2) with stromal cells (e.g., fibroblasts) (e.g., neonatal human fibroblasts), centrifuged into arrays of microwells (e.g., pyramidal microwells), and incubated for 2-3 days to promote self-assembly of the cells into multicellular hepatic aggregates. Hepatic aggregates are deemed acceptable for encapsulation after microscopic confirmation of compaction. In Step 6, the hepatocyte/fibroblast aggregates are encapsulated with a solution (e.g., a solution containing fibrinogen) that is polymerized (e.g., with thrombin). These encapsulation steps occur within a mold (e.g., a cylindrical mold) that controls the overall dimensions of the graft to be about 2 mm in thickness and with an outer diameter of 6 mm to 100 cm (e.g., 7 mm to 999 mm, 8 mm to 998 mm, 9 mm to 997 mm, 10 mm to 996 mm, 20 mm to 995 mm, 30 mm to 990 mm, 40 mm to 980 mm, 60 mm to 960 mm, 90 mm to 930 mm, 100 mm to 900 mm, 200 mm to 800 mm, 300 mm to 700 mm, 400 mm to 600 mm, or 500 mm). The thickness is controlled by the volume of cell-hydrogel suspension.

### DEFINITIONS

As used herein, the terms “implanting,” “implantation,” and the like, refer to directly giving a subject (e.g., a human subject) one or more engineered tissue construct at any effective implantation site, such as a site that is suitable for neovascularization. Exemplary implantation sites include the peritoneum (e.g., retroperitoneum), peritoneal cavity (e.g., omentum or mesentery), rectus abdominis muscle, abdominal oblique muscle, quadriceps femoris muscle, extraperitoneal fat, renal capsule, an extraperitoneal site, a site on the surface of the liver, and an extrapleural site, among others.

As used herein, the term a “subject” is a mammal. Mammals include, but are not limited to, domesticated animals (e.g., cows, sheep, cats, dogs, and horses), primates (e.g., humans and non-human primates such as monkeys), rabbits, deer, and rodents (e.g., mice and rats). In certain embodiments, the subject is a human.

As used herein, the terms “comprise,” “comprising,” “comprises,” and “comprised of” are synonymous with “include,” “including,” “includes” or “contain,” “containing,” “contains” and are inclusive or open-ended terms that specifies the presence of what follows e.g., a component and do not exclude or preclude the presence of additional, non-recited components, features, element, members, steps, known in the art or disclosed therein.

Whereas the terms “one or more” or “at least one,” such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

As used herein, the terms “amounts effect,” “effective amount,” “therapeutically effective amount,” and the like, when used in reference to an engineered tissue construct described herein, refer to a quantity of hepatocytes and stromal cells (e.g., fibroblasts) in the engineered tissue construct sufficient to, when implanted into the subject (e.g., a mammal e.g., a human) effect beneficial or desired results, such as clinical results. For example, in the context of treating hyperammonemia, such as in a patient having a urea cycle disorder, these terms refer to an amount of the hepatocyes and stromal cells (e.g., fibroblasts) sufficient to achieve a treatment response as compared to the response obtained without implantation of the engineered tissue construct of interest. An “effective amount,” “therapeutically effective amount,” and the like, of a engineered tissue construct of the present disclosure, also include an amount that results in a beneficial or desired result in a subject as compared to a control.

As used herein, the terms “treat” and “treatment” refer to therapeutic treatment, in which the object is to prevent or slow down (lessen) an undesired physiological change, such as the progression of hyperammonemia. Beneficial or desired clinical results include, but are not limited to, the reduction of ammonia, an improvement in a test of gallbladder ejection fraction, or the alleviation of hyperammonemia symptoms. The concentration of ammonia protein or the gallbladder ejection fraction may be determined using assays known in the art, for example, using a hepatobiliary iminodiacetic acid scan.

As used herein, the term “hyperammonemia” refers to a metabolic disturbance characterised by an excess of ammonia in the blood. As used herein, “hyperammonemia” includes primary hyperammonemia and secondary hyperammonemia as well as acquired hyperammonemia and congenital hyperammonemia.

As used herein, the term “urea cycle disorder” refers to any disorder that is caused by a defect or malfunction in the urea cycle. The urea cycle is a cycle of biochemical reactions that produces urea from ammonia, a product of protein catabolism. Specific types of urea cycle disorder include, but are not limited to, phosphate synthetase 1 (CPS1) deficiency, ornithine transcarbamylase (OTC) deficiency, argininosuccinate synthetase (ASS1) deficiency, argininosuccinate lyase (ASL) deficiency, arginase-1 (ARG1) deficiency, N-acetylglutamate synthetase (NAGS) deficiency, ornithine translocase (ORNT1) deficiency, and citrin deficiency. A urea cycle disorder may be characterized by an aberrant level of ammonia (e.g., an ammonia level of greater than or equal to about 90 μmol/L).

As used in the context of the present disclosure, an “engineered tissue construct” refers to a mixture of cultured hepatocytes (e.g., primary human hepatocytes) and stromal cells (e.g., fibroblasts) (e.g., neonatal foreskin stromal cells (e.g., fibroblasts). The relative volume of the engineered tissue construct may be between 0.5 mL to 5 L. In some embodiments, the engineered tissue construct further includes a biocompatible hydrogel scaffold (e.g., fibrin).

Cells can be from established cell lines or they can be primary cells, where “primary cells,” “primary cell lines,” and “primary cultures” are used interchangeably herein to refer to cells and cells cultures that have been derived from and allowed to grow in vitro for a limited number of passages, e.g., splitting, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. If the cells are primary cells, such cells can be harvested from an individual by any convenient method. For example, cells from tissues such as skin, muscle, bone marrow, spleen, liver, pancreas, lung, intestine, stomach, etc. are most conveniently harvested by biopsy. An appropriate solution can be used for dispersion or suspension of the harvested cells. Such solution will generally be a balanced salt solution, e.g., normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells can be used immediately, or they can be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures and thawed in a manner as commonly known in the art for thawing frozen cultured cells. For example, hepatocytes may be isolated by conventional methods (Berry et al., 1969, J. Cell Biol. 43:506-520) which can be adapted for human liver biopsy or autopsy material (e.g., to garner primary human hepatocytes).

As used herein, a hydrogel scaffold is considered “biocompatible” when is it does not exhibit toxicity when introduced into a subject (e.g., a human). In the context of the present disclosure, it is preferable that the biocompatible hydrogel scaffold does not exhibit toxicity towards the cells of the engineered tissue construct or when implanted in vivo in a subject (e.g., a human). Hepatotoxicity can be measured, for example, by determining hepatocytes apoptotic death rate (e.g., wherein an increase in apoptosis is indicative of hepatotoxicity), transaminase levels (e.g., wherein an increase in transaminase levels is indicative of hepatotoxicity), ballooning of the hepatocytes (e.g., wherein an increase in ballooning is indicative of hepatotoxicity), microvesicular steatosis in the hepatocytes (e.g., wherein an increase in steatosis is indicative of hepatotoxicity), biliary cells death rate (e.g., wherein an increase in biliary cells death rate is indicative of hepatotoxicity), γ-glutamyl transpeptidase (GGT) levels (e.g., wherein an increase in GGT levels is indicative of hepatotoxicity). A biocompatible hydrogel scaffold can include, but is not limited to, fibrin and heparin.

As used herein, the term “hydrogel” refers to a network of polymer chains that are hydrophilic in nature, such that the material absorbs a high volume of water or other aqueous solution. Hydrogels can include, for example, at least 70% v/v water, at least 80% v/v water, at least 90% v/v water, at least 95%, 96%, 97%, 98% and even 99% or greater v/v water (or other aqueous solution). Hydrogels can include natural or synthetic polymers, the polymeric network often featuring a high degree of crosslinking. Hydrogels also possess a degree of flexibility very similar to natural tissue, due to their significant water content. Hydrogels are particularly useful in tissue engineering applications as scaffolds for culturing cells. In certain embodiments, the hydrogels are made of biocompatible polymers.

The term “adherence material” is a material incorporated into the cell mixture disclosed herein to which a cell or microorganism has some affinity, such as a binding agent. The material can be incorporated, for example, into a hydrogel prior to implantation of the engineered cell mixture. The material and a cell or microorganism interact through any means including, for example, electrostatic or hydrophobic interactions, covalent binding or ionic attachment. The material may include, but is not limited to, antibodies, proteins, peptides, nucleic acids, peptide aptamers, nucleic acid aptamers, sugars, proteoglycans, or cellular receptors.

As used herein, the term “decompose,” refers to the physiological process of biochemical degradation, digestion, and/or break down of a molecule of interest (e.g., ammonia), to remove the molecule from the body (e.g., by renal clearance).

As used herein, the term “suitable for neovascularization” refers to conditions and/or environmental characteristics fit for the formation of new blood vessels. Generally, neovascularization means the formation of new blood vessels in injured tissue or in tissue not normally containing blood vessels or the formation of novel blood vessels (e.g., arterioles, venules, and capillaries) of a higher density than usual in said tissue. For example, a site that is suitable for neovascularization may have an existing microvessel density of greater than about 3.6 vessels/mm² (e.g., greater than about 3.7 vessels/mm², 3.8 vessels/mm², 3.9 vessels/mm², 4 vessels/mm², 4.1 vessels/mm², 4.2 vessels/mm², 4.3 vessels/mm², 4.4 vessels/mm², 4.5 vessels/mm², 5 vessels/mm², 6 vessels/mm², 7 vessels/mm², 8 vessels/mm², 9 vessels/mm², 10 vessels/mm², 50 vessels/mm², 100 vessels/mm², 200 vessels/mm², 300 vessels/mm², 400 vessels/mm², 500 vessels/mm², 1000 vessels/mm², 2000 vessels/mm², 3000 vessels/mm², 4000 vessels/mm², or 4500 vessels/mm²).

As used herein, the terms “liver function test” and “LFT” refer to a hepatic panel (e.g., a group of blood tests that provide information about the state of a patient's liver). A hepatic panel may include measurement of the level of gamma-glutamyl transferase, the level of alkaline phosphatase, the level of aspartate aminotransferase, the level of alanine aminotransferase, the level of albumin, the level of bilirubin, the prothrombin time, the activated partial thromboplastin time, or a combination thereof.

As used herein, the term “age-adjusted norms” refers to the process of a normalization of data by age, which is a technique that is used to allow populations of subjects to be compared when the age profiles of the populations are different. As used herein, the term “norm” refers to data that does not undergo a normalization by age, as populations of subjects across age profiles are similar.

As used herein, the term “level” refers to a level of a protein, as compared to a reference. The reference can be any useful reference, as defined herein. By a “decreased level” and an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an 15 increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, or ng/mL) or percentage relative to total protein in a sample.

By a “reference” is meant any useful reference used to compare protein levels related to hyperammonemia. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having hyperammonemia; a sample from a subject that has been treated for hyperammonemia; or a sample of a purified protein (e.g., any described herein) at a known normal concentration. By “reference standard or level” is meant a value or number derived from a reference sample. A “normal control value” is a pre-determined value indicative of non-hyperammonemic state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having hyperammonemia. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

### DETAILED DESCRIPTION

The present disclosure provides compositions and methods that can be used for treating hyperammonemia (e.g., in a subject having a urea cycle disorder, organic acidemia, congenital lactic acidosis, fatty acid oxidation defect, dibasic amino acid deficiencies, transient hyperammonemia, Reye syndrome, severe perinatal asphyxia, virus-associated hyperammonemia, infection, drug-induced hyperammonemia, liver disease, acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder, gastric bypass surgery, gastrointestinal bleeding, malnutrition, generalized seizures, organ transplantation, and/or renal failure) or reducing ammonia levels in a subject in need thereof. In accordance with the compositions and methods described herein, a subject (e.g., a human) having hyperammonemia may be implanted with one or more engineered tissue construct that includes a population of hepatocytes and an optional population of stromal cells (e.g., fibroblasts).

The present disclosure is based, at least in part, on the discovery that engineered tissue constructs, including hepatocytes and optionally stromal cells (e.g., fibroblasts), can be implanted and used for methods of treating hyperammonemia, thereby addressing the outstanding significant unmet medical need associated with the symptoms of hyperammonemia that exist in patients with a urea cycle disorder, organic acidemia, congenital lactic acidosis, fatty acid oxidation defect, dibasic amino acid deficiencies, transient hyperammonemia, Reye syndrome, severe perinatal asphyxia, virus-associated hyperammonemia, infection, drug-induced hyperammonemia, liver disease, acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder, gastric bypass surgery, gastrointestinal bleeding, malnutrition, generalized seizures, organ transplantation, or renal failure. The disclosure is also based, at least in part, on the inventors surprising discovery that implantation of engineered tissue constructs significantly lower levels of ammonia.

The sections that follow provide a description of engineered tissue construct. The following sections also describe various implantation sites and parameters for clinical monitoring following implantation of the engineered tissue construct that may be used in conjunction with the compositions and methods of the disclosure.

### Hyperammonemia

The urea cycle is a cycle of biochemical reactions that produces urea from ammonia, a product of protein catabolism. The urea cycle includes five key enzymes including carbamoyl phosphate synthetase 1 (CPS1), ornithine transcarbamoylase (OTC), argininosuccinate synthetase (ASS1), argininosuccinate lyase (ASL), and arginase 1 (ARG1), but also requires other enzymes, such as N-acetylglutamate synthetase (NAGS), and mitochondrial amino acid transporters, such as ornithine translocase (ORNT1) and citrin. The urea cycle mainly occurs in the mitochondria of liver cells. The urea produced by the liver enters the bloodstream where it travels to the kidneys and is ultimately excreted in urine. Genetic defects in any of the enzymes or transporters in the urea cycle can cause hyperammonemia (elevated blood ammonia), or the buildup of a cycle intermediate. Ammonia then reaches the brain through the blood, where it can cause cerebral edema, seizures, coma, long term disabilities in survivors, and/or death.

For example, the enzyme OTC is an important component of the urea cycle, catalyzing the formation of citrulline from carbamoyl phosphate and ornithine, which is critical to the removal of excess ammonia from the body. Deficiencies in the function of OTC can lead to hyperammonemia, which can damage the brain by a variety of mechanisms.

Hyperammonemia is an associated symptom of many diseases and disorders, including, for example, a urea cycle disorder (e.g., CPS1 deficiency, OTC deficiency, ASS1 deficiency, ASL deficiency, ARG1 deficiency, NAGS deficiency, ORNT1 deficiency, and citrin deficiency), organic acidemia, congenital lactic acidosis, fatty acid oxidation defect, dibasic amino acid deficiencies, transient hyperammonemia, Reye syndrome, severe perinatal asphyxia, virus-associated hyperammonemia, infection (a urinary tract infection or an infection caused by Proteus mirabilis, E. coli, or Klebsiella), drug-induced hyperammonemia (e.g., hyperammonemia is caused by the drug valproate acid, topiramate, carbamazepine, salicylate, sulfadiazine, a carbonic anhydrase inhibitor, a carbonic anhydrase inhibitor with valproate acid, or a chemotherapy), liver disease (e.g., biliary atresia, Alpha-1 antitrypsin deficiency, Wilson disease, cystic fibrosis, galactosemia, or tyrosinemia), acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder (e.g., multiple myeloma or acute leukemia), gastric bypass surgery, gastrointestinal bleeding, malnutrition, generalized seizures, organ transplantation, and renal failure, among others.

### Engineered Tissue Constructs

The engineered tissue constructs described herein include a population of hepatocytes and optionally a population of stromal cells (e.g., fibroblasts).

In another aspect, the cellular compositions are provided in the form of an aggregate of the hepatocyte and optional stromal cell populations. In some embodiments, the hepatocyte and optional stromal cell populations are admixed under conditions which cause the two cell populations to form aggregates. In some embodiments, the hepatocyte and optional stromal cell populations are admixed using tissue fabrication techniques. In some embodiments, the hepatocyte and optional stromal cell populations are co-cultured. In some embodiments, the hepatocyte and optional stromal cell populations are admixed using tissue fabrication techniques. In some embodiments, the hepatocyte and optional stromal cell populations are co-cultured. In some embodiments, the hepatocyte and optional stromal cell populations are cocultured by hanging drop, microwell molding, non-adhesive surfaces, spheroid suspension culture using a spinner flask, vertical wheel bioreactor, horizontal wheel bioreactor, or a microfluidic spheroid system.

In other aspects, the compositions provided herein can contain additional components, including but not limited to, growth factors, ligands, cytokines, drugs, etc. In some embodiments, the cell mixtures can include molecules which elicit additional microenvironmental cues such as small molecules or growth factors which stimulate or enhance proliferation and expansion of a cell population.

The properties of the cell aggregates of the present disclosure can be varied to suit a particular application. In certain embodiments, the density of the cell aggregates can be changed. In certain embodiments, cell aggregates of different diameters can be fabricated. In certain embodiments, the overall network organization of the one or more cell aggregates can be defined, for example, by the number, three-dimensional organization, alignment, diameters, density, and the like.

In certain embodiments, the engineered cell composition can contain one or more bioactive substances. Examples of bioactive substance(s) include, but are not limited to, hormones, neurotransmitters, growth factors, hormone, neurotransmitter or growth factor receptors, interferons, interleukins, chemokines, cytokines, colony stimulating factors, chemotactic factors, extracellular matrix components, and adhesion molecules, ligands and peptides; such as growth hormone, parathyroid hormone bone morphogenetic protein, transforming growth factor-alpha, TGF-beta1, TGF-beta2, fibroblast growth factor, granulocyte/macrophage colony stimulating factor, epidermal growth factor, platelet derived growth factor, insulin-like growth factor, scatter factor/hepatocyte growth factor, fibrin, dextran, matrix metalloproteinases, collagen, fibronectin, vitronectin, hyaluronic acid, an RGD-containing peptide or polypeptide, an angiopoietin and vascular endothelial cell growth factor.

In certain embodiments, the engineered cell mixtures disclosed herein include one or more adherence materials to facilitate maintenance of the desired phenotype of the grafted cells in vivo. The material may include, but is not limited to, antibodies, proteins, peptides, nucleic acids, peptide aptamers, nucleic acid aptamers, sugars, proteoglycans, or cellular receptors.

The type of adherence materials (e.g., extra-cellular matrix (ECM) materials, sugars, proteoglycans etc.) will be determined, in part, by the cell type or types (e.g., hepatocytes and stromal cells (e.g., fibroblasts)) to be cultured. ECM molecules found in a cell's native microenvironment are useful in maintaining the function of both primary cells, precursor cells, and/or cell lines.

In some embodiments, the engineered tissue constructs further includes a biocompatible hydrogel scaffold. For example, in some embodiments, the biocompatible scaffold is fibrin. In some embodiments, the biocompatible scaffold includes a synthetic heparin mimetic. In particular, the synthetic polymer of the invention may include an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin. Therefore, the synthetic polymer of the disclosure, as well as the hydrogel comprising the synthetic polymer described herein can bind various bioactive agents, e.g., growth factors, thereby preventing the bioactive agents from diffusing away and maintaining the bioactive agents at a high concentration locally, so that they can act on cells and promote various cell functions.

In some embodiments, the implant is from 0.1 mL to 5 L (e.g., 0.2 mL to 5 L, 0.3 mL to 5 L, 0.4 mL to 5 L, 0.5 mL to 5 L, 1 mL to 5 L, 5 mL to 5 L, 10 mL to 5 L, 100 mL to 5 L, 1 L to 5 L, 2 L to 5 L, 3 L to 5 L, or 4 L to 5 L) in volume. For example, in some embodiments, the implant is from 0.2 mL to 5 L in volume. In some embodiments, the implant is from 0.3 mL to 5 L in volume. In some embodiments, the implant is from 0.4 mL to 5 L in volume. In some embodiments, the implant is from 0.5 mL to 5 L in volume. In some embodiments, the implant is from 1 mL to 5 L in volume. In some embodiments, the implant is from 5 mL to 5 L in volume. In some embodiments, the implant is from 10 mL to 5 L in volume. In some embodiments, the implant is from 100 mL to 5 L in volume. In some embodiments, the implant is from 1 mL to 5 L in volume. In some embodiments, the implant is from 2 mL to 5 L in volume. In some embodiments, the implant is from 3 mL to 5 L in volume. In some embodiments, the implant is from 4 mL to 5 L in volume.

In some embodiments, the engineered tissue construct provides a microenvironment that promotes the persistence of hepatocyte survival for least three months (e.g., at least three months, at least four months, at least five months, at least six months, at least one year, at least five years, at least ten years, or the lifetime of a patient in which the engineered tissue construct is implanted into) in the subject. For example, in some embodiments, the engineered tissue construct provides a microenvironment that promotes the persistence of hepatocyte survival for least four months. In some embodiments, the engineered tissue construct provides a microenvironment that promotes the persistence of hepatocyte survival for least five months. In some embodiments, the engineered tissue construct provides a microenvironment that promotes the persistence of hepatocyte survival for least six months. In some embodiments, the engineered tissue construct provides a microenvironment that promotes the persistence of hepatocyte survival for least one year. In some embodiments, the engineered tissue construct provides a microenvironment that promotes the persistence of hepatocyte survival for least five years. In some embodiments, the engineered tissue construct provides a microenvironment that promotes the persistence of hepatocyte survival for least ten years.

In some embodiments, the engineered tissue construct may be any shape (e.g., cylindrical, square, or square with rounded corners).

In some embodiments, the engineered tissue construct has a serpentine topography (e.g., to increase surface area).

### Cell Populations

Cell populations are optimized to maintain the appropriate morphology, phenotype, and cellular function conducive to use in the methods of the disclosure. For example, primary human hepatocytes or neonatal foreskin fibroblasts (e.g., fibroblasts) can be isolated and/or pre-cultured under conditions optimized to ensure that the respective cells of choice initially have the desired morphology, phenotype and cellular function and, thus, are poised to maintain said morphology, phenotype and/or function in vivo following implantation of the engineered tissue constructs described herein.

### Hepatocytes

The engineered tissue constructs described herein include hepatocytes. In some embodiments, the hepatocytes are primary human hepatocytes (PHH). In some embodiments, the hepatocytes are derived from stem cells (e.g., induced pluripotent stem cells).

In some embodiments, the hepatocytes described herein are obtained by methods includes culturing and passaging the PHH to obtain a population of expanded PHH or obtaining the population of expanded PHH from a single PHH cell. In some embodiments, obtaining the population of expanded PHH includes culturing and passaging the PHH in an appropriate cell medium for human cells (e.g., for 3-120 days e.g., 4-119 days, 5-118 days, 10-117 days, 15-116 days, 20-115 days, 30-100 days, 40-90 days, 50-80 days, 60-70 days, or 65 days)). See e.g., U.S. Provisional Patent Application No. 63/271,441, incorporated herein by reference.

In some embodiments the engineered tissue construct includes a population of hepatocytes in an amount that is effective to treat hyperammonemia in a subject (e.g., a human).

In some embodiments the engineered tissue construct includes a population of hepatocytes in an amount that is effective to reduce ammonia levels in the subject (e.g., a human).

In some embodiments, the density of hepatocytes is 0.1 M/mL to 150 M/mL (e.g., 0.2 M/mL to 149 M/mL, 0.3 M/mL to 148 M/mL, 0.4 M/mL to 147 M/mL, 0.5 M/mL to 146 M/mL, 1 M/mL to 145 M/mL, 5 M/mL to 140 M/mL, 10 M/mL to 100 M/mL, 20 M/mL to 50 M/mL, or 30 M/mL to 40 M/mL). For example, in some embodiments, the density of hepatocytes is 0.2 M/mL to 149 M/mL. In some embodiments, the density of hepatocytes is 0.3 M/mL to 148 M/mL. In some embodiments, the density of hepatocytes is 0.4 M/mL to 147 M/mL. In some embodiments, the density of hepatocytes is 0.5 M/mL to 146 M/mL. In some embodiments, the density of hepatocytes is 1 M/mL to 145 M/mL. In some embodiments, the density of hepatocytes is 5 M/mL to 140 M/mL. In some embodiments, the density of hepatocytes is 10 M/mL to 100 M/mL. In some embodiments, the density of hepatocytes is 20 M/mL to 50 M/mL. In some embodiments, the density of hepatocytes is 30 M/mL to 40 M/mL. In some embodiments, the density of hepatocytes is 3 M/mL to 12 M/mL (e.g., 4 M/mL to 11 M/mL, 5 M/mL to 10 M/mL, 6 M/mL to 9 M/mL, or 7 M/mL to 8 M/mL). For example, in some embodiments, the density of hepatocytes is 4 M/mL to 11 M/mL. In some embodiments, the density of hepatocytes is 5 M/mL to 10 M/mL. In some embodiments, the density of hepatocytes is 6 M/mL to 9 M/mL. In some embodiments, the density of hepatocytes is 7 M/mL to 8 M/mL.

In some embodiments, the density of hepatocytes is 0.1 M/mL. In some embodiments, the density of hepatocytes is 0.2 M/mL. In some embodiments, the density of hepatocytes is 0.3 M/mL. In some embodiments, the density of hepatocytes is 0.4 M/mL. In some embodiments, the density of hepatocytes is 0.5 M/mL. In some embodiments, the density of hepatocytes is 0.6 M/mL. In some embodiments, the density of hepatocytes is 0.7 M/mL. In some embodiments, the density of hepatocytes is 0.8 M/mL. In some embodiments, the density of hepatocytes is 0.9 M/mL. In some embodiments, the density of hepatocytes is 1 M/mL. In some embodiments, the density of hepatocytes is 2 M/mL. In some embodiments, the density of hepatocytes is 3 M/mL. In some embodiments, the density of hepatocytes is 4 M/mL. In some embodiments, the density of hepatocytes is 5 M/mL. In some embodiments, the density of hepatocytes is 6 M/mL. In some embodiments, the density of hepatocytes is 7 M/mL. In some embodiments, the density of hepatocytes is 8 M/mL. In some embodiments, the density of hepatocytes is 9 M/mL. In some embodiments, the density of hepatocytes is 10 M/mL. In some embodiments, the density of hepatocytes is 11 M/mL. In some embodiments, the density of hepatocytes is 12 M/mL. In some embodiments, the density of hepatocytes is 13 M/mL. In some embodiments, the density of hepatocytes is 14 M/mL. In some embodiments, the density of hepatocytes is 15 M/mL. In some embodiments, the density of hepatocytes is 16 M/mL. In some embodiments, the density of hepatocytes is 17 M/mL. In some embodiments, the density of hepatocytes is 18 M/mL. In some embodiments, the density of hepatocytes is 19 M/mL. In some embodiments, the density of hepatocytes is 20 M/mL. In some embodiments, the density of hepatocytes is 21 M/mL. In some embodiments, the density of hepatocytes is 22 M/mL. In some embodiments, the density of hepatocytes is 23 M/mL. In some embodiments, the density of hepatocytes is 24 M/mL. In some embodiments, the density of hepatocytes is 25 M/mL. In some embodiments, the density of hepatocytes is 26 M/mL. In some embodiments, the density of hepatocytes is 27 M/mL. In some embodiments, the density of hepatocytes is 28 M/mL. In some embodiments, the density of hepatocytes is 29 M/mL. In some embodiments, the density of hepatocytes is 30 M/mL. In some embodiments, the density of hepatocytes is 31 M/mL. In some embodiments, the density of hepatocytes is 32 M/mL. In some embodiments, the density of hepatocytes is 33 M/mL. In some embodiments, the density of hepatocytes is 34 M/mL. In some embodiments, the density of hepatocytes is 35 M/mL. In some embodiments, the density of hepatocytes is 36 M/mL. In some embodiments, the density of hepatocytes is 37 M/mL. In some embodiments, the density of hepatocytes is 38 M/mL. In some embodiments, the density of hepatocytes is 39 M/mL. In some embodiments, the density of hepatocytes is 40 M/mL. In some embodiments, the density of hepatocytes is 41 M/mL. In some embodiments, the density of hepatocytes is 42 M/mL. In some embodiments, the density of hepatocytes is 43 M/mL. In some embodiments, the density of hepatocytes is 44 M/mL. In some embodiments, the density of hepatocytes is 45 M/mL. In some embodiments, the density of hepatocytes is 46 M/mL. In some embodiments, the density of hepatocytes is 47 M/mL. In some embodiments, the density of hepatocytes is 48 M/mL. In some embodiments, the density of hepatocytes is 49 M/mL. In some embodiments, the density of hepatocytes is 50 M/mL. In some embodiments, the density of hepatocytes is 60 M/mL. In some embodiments, the density of hepatocytes is 70 M/mL. In some embodiments, the density of hepatocytes is 80 M/mL. In some embodiments, the density of hepatocytes is 90 M/mL. In some embodiments, the density of hepatocytes is 100 M/mL. In some embodiments, the density of hepatocytes is 150 M/mL.

In some embodiments, the hepatocytes have a quantifiable potency (e.g., an ability to decompose ammonia in a quantifiable range).

### I. Percentage of Hepatocytes Compared to Total Liver Mass

In some embodiments, the engineered tissue construct includes a population of hepatocytes in an amount that is equivalent to 0.5% to 30% (e.g., 0.5% to 30%, 0.6% to 30%, 0.7% to 30%, 0.8% to 30%, 0.9% to 30%, 1% to 30%, 2% to 30%, 3% to 30%, 4% to 30%, 5% to 30%, 10% to 30%, or 20% to 30%) of the total liver mass of the subject. For example, in some embodiments, the engineered tissue construct includes a population of hepatocytes in an amount that is equivalent to 0.6% to 30% of the total liver mass of the subject. In some embodiments, the engineered tissue construct includes a population of hepatocytes in an amount that is equivalent to 0.7% to 30% of the total liver mass of the subject. In some embodiments, the engineered tissue construct includes a population of hepatocytes in an amount that is equivalent to 0.8% to 30% of the total liver mass of the subject. In some embodiments, the engineered tissue construct includes a population of hepatocytes in an amount that is equivalent to 0.9% to 30% of the total liver mass of the subject. In some embodiments, the engineered tissue construct includes a population of hepatocytes in an amount that is equivalent to 1% to 30% of the total liver mass of the subject. In some embodiments, the engineered tissue construct includes a population of hepatocytes in an amount that is equivalent to 2% to 30% of the total liver mass of the subject. In some embodiments, the engineered tissue construct includes a population of hepatocytes in an amount that is equivalent to 3% to 30% of the total liver mass of the subject. In some embodiments, the engineered tissue construct includes a population of hepatocytes in an amount that is equivalent to 4% to 30% of the total liver mass of the subject. In some embodiments, the engineered tissue construct includes a population of hepatocytes in an amount that is equivalent to 5% to 30% of the total liver mass of the subject. In some embodiments, the engineered tissue construct includes a population of hepatocytes in an amount that is equivalent to 6% to 30% of the total liver mass of the subject. In some embodiments, the engineered tissue construct includes a population of hepatocytes in an amount that is equivalent to 7% to 30% of the total liver mass of the subject. In some embodiments, the engineered tissue construct includes a population of hepatocytes in an amount that is equivalent to 8% to 30% of the total liver mass of the subject. In some embodiments, the engineered tissue construct includes a population of hepatocytes in an amount that is equivalent to 9% to 30% of the total liver mass of the subject. In some embodiments, the engineered tissue construct includes a population of hepatocytes in an amount that is equivalent to 10% to 30% of the total liver mass of the subject. In some embodiments, the engineered tissue construct includes a population of hepatocytes in an amount that is equivalent to 11% to 30% of the total liver mass of the subject. In some embodiments, the engineered tissue construct includes a population of hepatocytes in an amount that is equivalent to 12% to 30% of the total liver mass of the subject. In some embodiments, the engineered tissue construct includes a population of hepatocytes in an amount that is equivalent to 13% to 30% of the total liver mass of the subject. In some embodiments, the engineered tissue construct includes a population of hepatocytes in an amount that is equivalent to 14% to 30% of the total liver mass of the subject. In some embodiments, the engineered tissue construct includes a population of hepatocytes in an amount that is equivalent to 15% to 30% of the total liver mass of the subject. In some embodiments, the engineered tissue construct includes a population of hepatocytes in an amount that is equivalent to 16% to 30% of the total liver mass of the subject. In some embodiments, the engineered tissue construct includes a population of hepatocytes in an amount that is equivalent to 17% to 30% of the total liver mass of the subject. In some embodiments, the engineered tissue construct includes a population of hepatocytes in an amount that is equivalent to 18% to 30% of the total liver mass of the subject. In some embodiments, the engineered tissue construct includes a population of hepatocytes in an amount that is equivalent to 19% to 30% of the total liver mass of the subject. In some embodiments, the engineered tissue construct includes a population of hepatocytes in an amount that is equivalent to 20% to 30% of the total liver mass of the subject. In some embodiments, the engineered tissue construct includes a population of hepatocytes in an amount that is equivalent to 21% to 30% of the total liver mass of the subject. In some embodiments, the engineered tissue construct includes a population of hepatocytes in an amount that is equivalent to 22% to 30% of the total liver mass of the subject. In some embodiments, the engineered tissue construct includes a population of hepatocytes in an amount that is equivalent to 23% to 30% of the total liver mass of the subject. In some embodiments, the engineered tissue construct includes a population of hepatocytes in an amount that is equivalent to 24% to 30% of the total liver mass of the subject. In some embodiments, the engineered tissue construct includes a population of hepatocytes in an amount that is equivalent to 25% to 30% of the total liver mass of the subject. In some embodiments, the engineered tissue construct includes a population of hepatocytes in an amount that is equivalent to 26% to 30% of the total liver mass of the subject. In some embodiments, the engineered tissue construct includes a population of hepatocytes in an amount that is equivalent to 27% to 30% of the total liver mass of the subject. In some embodiments, the engineered tissue construct includes a population of hepatocytes in an amount that is equivalent to 28% to 30% of the total liver mass of the subject. In some embodiments, the engineered tissue construct includes a population of hepatocytes in an amount that is equivalent to 29% to 30% of the total liver mass of the subject.

In some embodiments, the engineered tissue construct includes a population of hepatocytes in an amount that is equivalent to 0.5% of the total liver mass of the subject. For example, in some embodiments, the engineered tissue construct includes a population of hepatocytes in an amount that is equivalent to 0.6% of the total liver mass of the subject. In some embodiments, the engineered tissue construct includes a population of hepatocytes in an amount that is equivalent to 0.7% of the total liver mass of the subject. In some embodiments, the engineered tissue construct includes a population of hepatocytes in an amount that is equivalent to 0.8% of the total liver mass of the subject. In some embodiments, the engineered tissue construct includes a population of hepatocytes in an amount that is equivalent to 0.9% of the total liver mass of the subject. In some embodiments, the engineered tissue construct includes a population of hepatocytes in an amount that is equivalent to 1% of the total liver mass of the subject. In some embodiments, the engineered tissue construct includes a population of hepatocytes in an amount that is equivalent to 2% of the total liver mass of the subject. In some embodiments, the engineered tissue construct includes a population of hepatocytes in an amount that is equivalent to 3% of the total liver mass of the subject. In some embodiments, the engineered tissue construct includes a population of hepatocytes in an amount that is equivalent to 4% of the total liver mass of the subject. In some embodiments, the engineered tissue construct includes a population of hepatocytes in an amount that is equivalent to 5% of the total liver mass of the subject. In some embodiments, the engineered tissue construct includes a population of hepatocytes in an amount that is equivalent to 6% of the total liver mass of the subject. In some embodiments, the engineered tissue construct includes a population of hepatocytes in an amount that is equivalent to 7% of the total liver mass of the subject. In some embodiments, the engineered tissue construct includes a population of hepatocytes in an amount that is equivalent to 8% of the total liver mass of the subject. In some embodiments, the engineered tissue construct includes a population of hepatocytes in an amount that is equivalent to 9% of the total liver mass of the subject. In some embodiments, the engineered tissue construct includes a population of hepatocytes in an amount that is equivalent to 10% of the total liver mass of the subject. In some embodiments, the engineered tissue construct includes a population of hepatocytes in an amount that is equivalent to 11% of the total liver mass of the subject. In some embodiments, the engineered tissue construct includes a population of hepatocytes in an amount that is equivalent to 12% of the total liver mass of the subject. In some embodiments, the engineered tissue construct includes a population of hepatocytes in an amount that is equivalent to 13% of the total liver mass of the subject. In some embodiments, the engineered tissue construct includes a population of hepatocytes in an amount that is equivalent to 14% of the total liver mass of the subject. In some embodiments, the engineered tissue construct includes a population of hepatocytes in an amount that is equivalent to 15% of the total liver mass of the subject. In some embodiments, the engineered tissue construct includes a population of hepatocytes in an amount that is equivalent to 16% of the total liver mass of the subject. In some embodiments, the engineered tissue construct includes a population of hepatocytes in an amount that is equivalent to 17% of the total liver mass of the subject. In some embodiments, the engineered tissue construct includes a population of hepatocytes in an amount that is equivalent to 18% of the total liver mass of the subject. In some embodiments, the engineered tissue construct includes a population of hepatocytes in an amount that is equivalent to 19% of the total liver mass of the subject. In some embodiments, the engineered tissue construct includes a population of hepatocytes in an amount that is equivalent to 20% of the total liver mass of the subject. In some embodiments, the engineered tissue construct includes a population of hepatocytes in an amount that is equivalent to 21% of the total liver mass of the subject. In some embodiments, the engineered tissue construct includes a population of hepatocytes in an amount that is equivalent to 22% of the total liver mass of the subject. In some embodiments, the engineered tissue construct includes a population of hepatocytes in an amount that is equivalent to 23% of the total liver mass of the subject. In some embodiments, the engineered tissue construct includes a population of hepatocytes in an amount that is equivalent to 24% of the total liver mass of the subject. In some embodiments, the engineered tissue construct includes a population of hepatocytes in an amount that is equivalent to 25% of the total liver mass of the subject. In some embodiments, the engineered tissue construct includes a population of hepatocytes in an amount that is equivalent to 26% of the total liver mass of the subject. In some embodiments, the engineered tissue construct includes a population of hepatocytes in an amount that is equivalent to 27% of the total liver mass of the subject. In some embodiments, the engineered tissue construct includes a population of hepatocytes in an amount that is equivalent to 28% of the total liver mass of the subject. In some embodiments, the engineered tissue construct includes a population of hepatocytes in an amount that is equivalent to 29% of the total liver mass of the subject.

### II. Percentage of Hepatocytes Compared to Mass of Liver Reserve

In some embodiments, the engineered tissue construct includes a population of hepatocytes in an amount that is equivalent to 0.5% to 20% (e.g., 0.5% to 20%, 0.6% to 20%, 0.7% to 20%, 0.8% to 20%, 0.9% to 20%, 1% to 20%, 2% to 20%, 3% to 20%, 4% to 20%, 5% to 20%, or 10% to 20%) of the mass of the liver reserve of the subject. For example, in some embodiments, the engineered tissue construct includes a population of hepatocytes in an amount that is equivalent to 0.6% to 20% of the mass of the liver reserve of the subject. In some embodiments, the engineered tissue construct includes a population of hepatocytes in an amount that is equivalent to 0.7% to 20% of the mass of the liver reserve of the subject. In some embodiments, the engineered tissue construct includes a population of hepatocytes in an amount that is equivalent to 0.8% to 20% of the mass of the liver reserve of the subject.

In some embodiments, the engineered tissue construct includes a population of hepatocytes in an amount that is equivalent to 0.9% to 20% of the mass of the liver reserve of the subject. In some embodiments, the engineered tissue construct includes a population of hepatocytes in an amount that is equivalent to 1% to 20% of the mass of the liver reserve of the subject. In some embodiments, the engineered tissue construct includes a population of hepatocytes in an amount that is equivalent to 2% to 20% of the mass of the liver reserve of the subject. In some embodiments, the engineered tissue construct includes a population of hepatocytes in an amount that is equivalent to 3% to 20% of the mass of the liver reserve of the subject. In some embodiments, the engineered tissue construct includes a population of hepatocytes in an amount that is equivalent to 4% to 20% of the mass of the liver reserve of the subject. In some embodiments, the engineered tissue construct includes a population of hepatocytes in an amount that is equivalent to 5% to 20% of the mass of the liver reserve of the subject. In some embodiments, the engineered tissue construct includes a population of hepatocytes in an amount that is equivalent to 6% to 20% of the mass of the liver reserve of the subject. In some embodiments, the engineered tissue construct includes a population of hepatocytes in an amount that is equivalent to 7% to 20% of the mass of the liver reserve of the subject. In some embodiments, the engineered tissue construct includes a population of hepatocytes in an amount that is equivalent to 8% to 20% of the mass of the liver reserve of the subject. In some embodiments, the engineered tissue construct includes a population of hepatocytes in an amount that is equivalent to 9% to 20% of the mass of the liver reserve of the subject. In some embodiments, the engineered tissue construct includes a population of hepatocytes in an amount that is equivalent to 10% to 20% of the mass of the liver reserve of the subject. In some embodiments, the engineered tissue construct includes a population of hepatocytes in an amount that is equivalent to 11% to 20% of the mass of the liver reserve of the subject. In some embodiments, the engineered tissue construct includes a population of hepatocytes in an amount that is equivalent to 12% to 20% of the mass of the liver reserve of the subject. In some embodiments, the engineered tissue construct includes a population of hepatocytes in an amount that is equivalent to 13% to 20% of the mass of the liver reserve of the subject. In some embodiments, the engineered tissue construct includes a population of hepatocytes in an amount that is equivalent to 14% to 20% of the mass of the liver reserve of the subject. In some embodiments, the engineered tissue construct includes a population of hepatocytes in an amount that is equivalent to 15% to 20% of the mass of the liver reserve of the subject.

In some embodiments, the engineered tissue construct includes a population of hepatocytes in an amount that is equivalent to 16% to 20% of the mass of the liver reserve of the subject. In some embodiments, the engineered tissue construct includes a population of hepatocytes in an amount that is equivalent to 17% to 20% of the mass of the liver reserve of the subject. In some embodiments, the engineered tissue construct includes a population of hepatocytes in an amount that is equivalent to 18% to 20% of the mass of the liver reserve of the subject. In some embodiments, the engineered tissue construct includes a population of hepatocytes in an amount that is equivalent to 19% to 20% of the mass of the liver reserve of the subject.

In some embodiments, the engineered tissue construct includes a population of hepatocytes in an amount that is equivalent to 0.5% of the mass of the liver reserve of the subject. For example, in some embodiments, the engineered tissue construct includes a population of hepatocytes in an amount that is equivalent to 0.6% of the mass of the liver reserve of the subject. In some embodiments, the engineered tissue construct includes a population of hepatocytes in an amount that is equivalent to 0.7% of the mass of the liver reserve of the subject. In some embodiments, the engineered tissue construct includes a population of hepatocytes in an amount that is equivalent to 0.8% of the mass of the liver reserve of the subject. In some embodiments, the engineered tissue construct includes a population of hepatocytes in an amount that is equivalent to 0.9% of the mass of the liver reserve of the subject. In some embodiments, the engineered tissue construct includes a population of hepatocytes in an amount that is equivalent to 1% of the mass of the liver reserve of the subject. In some embodiments, the engineered tissue construct includes a population of hepatocytes in an amount that is equivalent to 2% of the mass of the liver reserve of the subject. In some embodiments, the engineered tissue construct includes a population of hepatocytes in an amount that is equivalent to 3% of the mass of the liver reserve of the subject. In some embodiments, the engineered tissue construct includes a population of hepatocytes in an amount that is equivalent to 4% of the mass of the liver reserve of the subject. In some embodiments, the engineered tissue construct includes a population of hepatocytes in an amount that is equivalent to 5% of the mass of the liver reserve of the subject. In some embodiments, the engineered tissue construct includes a population of hepatocytes in an amount that is equivalent to 6% of the mass of the liver reserve of the subject. In some embodiments, the engineered tissue construct includes a population of hepatocytes in an amount that is equivalent to 7% of the mass of the liver reserve of the subject. In some embodiments, the engineered tissue construct includes a population of hepatocytes in an amount that is equivalent to 8% of the mass of the liver reserve of the subject. In some embodiments, the engineered tissue construct includes a population of hepatocytes in an amount that is equivalent to 9% of the mass of the liver reserve of the subject. In some embodiments, the engineered tissue construct includes a population of hepatocytes in an amount that is equivalent to 10% of the mass of the liver reserve of the subject. In some embodiments, the engineered tissue construct includes a population of hepatocytes in an amount that is equivalent to 11% of the mass of the liver reserve of the subject. In some embodiments, the engineered tissue construct includes a population of hepatocytes in an amount that is equivalent to 12% of the mass of the liver reserve of the subject. In some embodiments, the engineered tissue construct includes a population of hepatocytes in an amount that is equivalent to 13% of the mass of the liver reserve of the subject. In some embodiments, the engineered tissue construct includes a population of hepatocytes in an amount that is equivalent to 14% of the mass of the liver reserve of the subject. In some embodiments, the engineered tissue construct includes a population of hepatocytes in an amount that is equivalent to 15% of the mass of the liver reserve of the subject. In some embodiments, the engineered tissue construct includes a population of hepatocytes in an amount that is equivalent to 16% of the mass of the liver reserve of the subject. In some embodiments, the engineered tissue construct includes a population of hepatocytes in an amount that is equivalent to 17% of the mass of the liver reserve of the subject. In some embodiments, the engineered tissue construct includes a population of hepatocytes in an amount that is equivalent to 18% of the mass of the liver reserve of the subject. In some embodiments, the engineered tissue construct includes a population of hepatocytes in an amount that is equivalent to 19% of the mass of the liver reserve of the subject. In some embodiments, the engineered tissue construct includes a population of hepatocytes in an amount that is equivalent to 20% of the mass of the liver reserve of the subject.

### III. Number of Hepatocytes in an Engineered Tissue Construct

In some embodiments, the engineered tissue construct includes a population of hepatocytes in an amount of from about 3×10⁵ to 1.8×10¹¹ (e.g., from about 4×10⁵ to about 1.8×10¹¹, from about 5×10⁵ to about 1.8×10¹¹, from about 6×10⁵ to about 1.8×10¹¹, from about 7×10⁵ to about 1.8×10¹¹, from about 8×10⁵ to about 1.8×10¹¹, from about 9×10⁵ to about 1.8×10¹¹, from about 1×10⁶ to about 1.8×10¹¹, from about 2×10⁶ to about 1.8×10¹¹, from about 3×10⁶ to about 1.8×10¹¹, from about 4×10⁶ to about 1.8×10¹¹, from about 5×10⁶ to about 1.8×10¹¹, from about 6×10⁶ to about 1.8×10¹¹, from about 7×10⁶ to about 1.8×10¹¹, from about 8×10⁶ to about 1.8×10¹¹, from about 9×10⁶ to about 1.8×10¹¹, from about 1×10⁷ to about 1.8×10¹¹, from about 2×10⁷ to about 1.8×10¹¹, from about 3×10⁷ to about 1.8×10¹¹, from about 4×10⁷ to about 1.8×10¹¹, from about 5×10⁷ to about 1.8×10¹¹, from about 6×10⁷ to about 1.8×10¹¹, from about 7×10⁷ to about 1.8×10¹¹, from about 8×10⁷ to about 1.8×10¹¹, from about 9×10⁷ to about 1.8×10¹¹, from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8×10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ toto at 1.8×10¹¹, from about 7×10⁹ to about 1.8×10¹¹, from about 8×10⁹ to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes. For example, in some embodiments, the engineered tissue construct includes a population of hepatocytes in an amount of from about 4×10⁵ to about 1.8×10¹¹ hepatocytes. In some embodiments, the engineered tissue construct includes a population of hepatocytes in an amount of from about 5×10⁵ to about 1.8×10¹¹ hepatocytes. In some embodiments, the engineered tissue construct includes a population of hepatocytes in an amount of from about 6×10⁵ to about 1.8×10¹¹ hepatocytes. In some embodiments, the engineered tissue construct includes a population of hepatocytes in an amount of from about 7×10⁵ to about 1.8×10¹¹ hepatocytes. In some embodiments, the engineered tissue construct includes a population of hepatocytes in an amount of from about 8×10⁵ to about 1.8×10¹¹ hepatocytes. In some embodiments, the engineered tissue construct includes a population of hepatocytes in an amount of from about 9×10⁵ to about 1.8×10¹¹ hepatocytes. In some embodiments, the engineered tissue construct includes a population of hepatocytes in an amount of from about 1×10⁶ to about 1.8×10¹¹ hepatocytes.

In some embodiments, the engineered tissue construct includes a population of hepatocytes in an amount of from about 2×10⁶ to about 1.8×10¹¹ hepatocytes. In some embodiments, the engineered tissue construct includes a population of hepatocytes in an amount of from about 3×10⁶ to about 1.8×10¹¹ hepatocytes. In some embodiments, the engineered tissue construct includes a population of hepatocytes in an amount of from about 4×10⁶ to about 1.8×10¹¹ hepatocytes. In some embodiments, the engineered tissue construct includes a population of hepatocytes in an amount of from about 5×10⁶ to about 1.8×10¹¹ hepatocytes. In some embodiments, the engineered tissue construct includes a population of hepatocytes in an amount of from about 6×10⁶ to about 1.8×10¹¹ hepatocytes. In some embodiments, the engineered tissue construct includes a population of hepatocytes in an amount of from about 7×10⁶ to about 1.8×10¹¹ hepatocytes. In some embodiments, the engineered tissue construct includes a population of hepatocytes in an amount of from about 8×10⁶ to about 1.8×10¹¹ hepatocytes. In some embodiments, the engineered tissue construct includes a population of hepatocytes in an amount of from about 9×10⁶ to about 1.8×10¹¹ hepatocytes. In some embodiments, the engineered tissue construct includes a population of hepatocytes in an amount of from about 1×10⁷ to about 1.8×10¹¹ hepatocytes. In some embodiments, the engineered tissue construct includes a population of hepatocytes in an amount of from about 2×10⁷ to about 1.8×10¹¹ hepatocytes. In some embodiments, the engineered tissue construct includes a population of hepatocytes in an amount of from about 3×10⁷ to about 1.8×10¹¹ hepatocytes. In some embodiments, the engineered tissue construct includes a population of hepatocytes in an amount of from about 4×10⁷ to about 1.8×10¹¹ hepatocytes. In some embodiments, the engineered tissue construct includes a population of hepatocytes in an amount of from about 5×10⁷ to about 1.8×10¹¹ hepatocytes. In some embodiments, the engineered tissue construct includes a population of hepatocytes in an amount of from about 6×10⁷ to about 1.8×10¹¹ hepatocytes. In some embodiments, the engineered tissue construct includes a population of hepatocytes in an amount of from about 7×10⁷ to about 1.8×10¹¹ hepatocytes. In some embodiments, the engineered tissue construct includes a population of hepatocytes in an amount of from about 8×10⁷ to about 1.8×10¹¹ hepatocytes. In some embodiments, the engineered tissue construct includes a population of hepatocytes in an amount of from about 9×10⁷ to about 1.8×10¹¹ hepatocytes. In some embodiments, the engineered tissue construct includes a population of hepatocytes in an amount of from about 1×10⁸ to about 1.8×10¹¹ hepatocytes. In some embodiments, the engineered tissue construct includes a population of hepatocytes in an amount of from about 2×10⁸ to about 1.8×10¹¹ hepatocytes. In some embodiments, the engineered tissue construct includes a population of hepatocytes in an amount of from about 3×10⁸ to about 1.8×10¹¹ hepatocytes. In some embodiments, the engineered tissue construct includes a population of hepatocytes in an amount of from about 4×10⁸ to about 1.8×10¹¹ hepatocytes. In some embodiments, the engineered tissue construct includes a population of hepatocytes in an amount of from about 5×10⁸ to about 1.8×10¹¹ hepatocytes. In some embodiments, the engineered tissue construct includes a population of hepatocytes in an amount of from about 6×10⁸ to about 1.8×10¹¹ hepatocytes. In some embodiments, the engineered tissue construct includes a population of hepatocytes in an amount of from about 7×10⁸ to about 1.8×10¹¹ hepatocytes. In some embodiments, the engineered tissue construct includes a population of hepatocytes in an amount of from about 8×10⁸ to about 1.8×10¹¹ hepatocytes. In some embodiments, the engineered tissue construct includes a population of hepatocytes in an amount of from about 9×10⁸ to about 1.8×10¹¹ hepatocytes. In some embodiments, the engineered tissue construct includes a population of hepatocytes in an amount of from about 1×10⁹ to about 1.8×10¹¹ hepatocytes. In some embodiments, the engineered tissue construct includes a population of hepatocytes in an amount of from about 2×10⁹ to about 1.8×10¹¹ hepatocytes. In some embodiments, the engineered tissue construct includes a population of hepatocytes in an amount of from about 3×10⁹ to about 1.8×10¹¹ hepatocytes. In some embodiments, the engineered tissue construct includes a population of hepatocytes in an amount of from about 4×10⁹ to about 1.8×10¹¹ hepatocytes. In some embodiments, the engineered tissue construct includes a population of hepatocytes in an amount of from about 5×10⁹ to about 1.8×10¹¹ hepatocytes. In some embodiments, the engineered tissue construct includes a population of hepatocytes in an amount of from about 6×10⁹ to about 1.8×10¹¹ hepatocytes. In some embodiments, the engineered tissue construct includes a population of hepatocytes in an amount of from about 7×10⁹ to about 1.8×10¹¹ hepatocytes. In some embodiments, the engineered tissue construct includes a population of hepatocytes in an amount of from about 8×10⁹ to about 1.8×10¹¹ hepatocytes. In some embodiments, the engineered tissue construct includes a population of hepatocytes in an amount of from about 9×10⁹ to about 1.8×10¹¹ hepatocytes. In some embodiments, the engineered tissue construct includes a population of hepatocytes in an amount of from about 1×10¹⁰ to about 1.8×10¹¹ hepatocytes. In some embodiments, the engineered tissue construct includes a population of hepatocytes in an amount of from about 2×10¹⁰ to about 1.8×10¹¹ hepatocytes. In some embodiments, the engineered tissue construct includes a population of hepatocytes in an amount of from about 3×10¹⁰ to about 1.8×10¹¹ hepatocytes. In some embodiments, the engineered tissue construct includes a population of hepatocytes in an amount of from about 4×10¹⁰ to about 1.8×10¹¹ hepatocytes. In some embodiments, the engineered tissue construct includes a population of hepatocytes in an amount of from about 5×10¹⁰ to about 1.8×10¹¹ hepatocytes. In some embodiments, the engineered tissue construct includes a population of hepatocytes in an amount of from about 6×10¹⁰ to about 1.8×10¹¹ hepatocytes. In some embodiments, the engineered tissue construct includes a population of hepatocytes in an amount of from about 7×10¹⁰ to about 1.8×10¹¹ hepatocytes. In some embodiments, the engineered tissue construct includes a population of hepatocytes in an amount of from about 8×10¹⁰ to about 1.8×10¹¹ hepatocytes. In some embodiments, the engineered tissue construct includes a population of hepatocytes in an amount of from about 9×10¹⁰ to about 1.8×10¹¹ hepatocytes. In some embodiments, the engineered tissue construct includes a population of hepatocytes in an amount of from about 1×10¹¹ to about 1.8×10¹¹ hepatocytes.

In some embodiments, the engineered tissue construct includes a population of hepatocytes in an amount of about 3×10⁵ hepatocytes. In some embodiments, the engineered tissue construct includes a population of hepatocytes in an amount of about 4×10⁵ hepatocytes. In some embodiments, the engineered tissue construct includes a population of hepatocytes in an amount of about 5×10⁵ hepatocytes. In some embodiments, the engineered tissue construct includes a population of hepatocytes in an amount of about 6×10⁵ hepatocytes. In some embodiments, the engineered tissue construct includes a population of hepatocytes in an amount of about 7×10⁵ hepatocytes. In some embodiments, the engineered tissue construct includes a population of hepatocytes in an amount of about 8×10⁵ hepatocytes. In some embodiments, the engineered tissue construct includes a population of hepatocytes in an amount of about 9×10⁵ hepatocytes. In some embodiments, the engineered tissue construct includes a population of hepatocytes in an amount of about 1×10⁶ hepatocytes. In some embodiments, the engineered tissue construct includes a population of hepatocytes in an amount of about 2×10⁶ hepatocytes. In some embodiments, the engineered tissue construct includes a population of hepatocytes in an amount of about 3×10⁶ hepatocytes. In some embodiments, the engineered tissue construct includes a population of hepatocytes in an amount of about 4×10⁶ hepatocytes. In some embodiments, the engineered tissue construct includes a population of hepatocytes in an amount of about 5×10⁶ hepatocytes. In some embodiments, the engineered tissue construct includes a population of hepatocytes in an amount of about 6×10⁶ hepatocytes. In some embodiments, the engineered tissue construct includes a population of hepatocytes in an amount of about 7×10⁶ hepatocytes. In some embodiments, the engineered tissue construct includes a population of hepatocytes in an amount of about 8×10⁶ hepatocytes. In some embodiments, the engineered tissue construct includes a population of hepatocytes in an amount of about 9×10⁶ hepatocytes. In some embodiments, the engineered tissue construct includes a population of hepatocytes in an amount of about 1×10⁷ hepatocytes. In some embodiments, the engineered tissue construct includes a population of hepatocytes in an amount of about 2×10⁷ hepatocytes. In some embodiments, the engineered tissue construct includes a population of hepatocytes in an amount of about 3×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.8×10¹¹ hepatocytes.

IIIa. Age-Dependent Number of Hepatocytes in an Engineered Tissue Construct
IIIai. Neonate (e.g., 0-30 Days of Age)

In some embodiments, the engineered tissue construct is implanted in a neonate and includes a population of hepatocytes in an amount of from about 3×10⁵ to about 3×10¹⁰ (e.g., from about 4×10⁵ to about 3×10¹⁰, from about 5×10⁵ to about 3×10¹⁰, from about 6×10⁵ to about 3×10¹⁰, from about 7×10⁵ to about 3×10¹⁰, from about 8×10⁵ to about 3×10¹⁰, from about 9×10⁵ to about 3×10¹⁰, from about 1×10⁶ to about 3×10¹⁰, from about 2×10⁶ to about 3×10¹⁰, from about 3×10⁶ to about 3×10¹⁰, from about 4×10⁶ to about 3×10¹⁰, from about 5×10⁶ to about 3×10¹⁰, from about 6×10⁶ to about 3×10¹⁰, from about 7×10⁶ to about 3×10¹⁰, from about 8×10⁶ to about 3×10¹⁰, from about 9×10⁶ to about 3×10¹⁰, from about 1×10⁷ to about 3×10¹⁰, from about 2×10⁷ to about 3×10¹⁰, from about 3×10⁷ to about 3×10¹⁰, from about 4×10⁷ to about 3×10¹⁰, from about 5×10⁷ to about 3×10¹⁰, from about 6×10⁷ to about 3×10¹⁰, from about 7×10⁷ to about 3×10¹⁰, from about 8×10⁷ to about 3×10¹⁰, from about 9×10⁷ to about 3×10¹⁰, from about 1×10⁸ to about 3×10¹⁰, from about 2×10⁸ to about 3×10¹⁰, from about 3×10⁸ to about 3×10¹⁰, from about 4×10⁸ to about 3×10¹⁰, from about 5×10⁸ to about 3×10¹⁰, from about 6×10⁸ to about 3×10¹⁰, from about 7×10⁸ to about 3×10¹⁰, from about 8×10⁸ to about 3×10¹⁰, from about 9×10⁸ to about 3×10¹⁰, from about 1×10⁹ to about 3×10¹⁰, from about 2×10⁹ to about 3×10¹⁰, from about 3×10⁹ to about 3×10¹⁰, from about 4×10⁹ to about 3×10¹⁰, from about 5×10⁹ to about 3×10¹⁰, from about 6×10⁹ to about 3×10¹⁰, from about 7×10⁹ to about 3×10¹⁰, from about 8×10⁹ to about 3×10¹⁰, from about 9×10⁹ to about 3×10¹⁰, from about 1×10¹⁰ to about 3×10¹⁰, or from about 2×10¹⁰ to about 3×10¹⁰) hepatocytes. For example, in some embodiments, the engineered tissue construct includes a population of hepatocytes in an amount of from about 4×10⁵ to about 3×10¹⁰ hepatocytes. In some embodiments, the engineered tissue construct includes a population of hepatocytes in an amount of from about 5×10⁵ to about 3×10¹⁰ hepatocytes. In some embodiments, the engineered tissue construct includes a population of hepatocytes in an amount of from about 6×10⁵ to about 3×10¹⁰ hepatocytes. In some embodiments, the engineered tissue construct includes a population of hepatocytes in an amount of from about 7×10⁵ to about 3×10¹⁰ hepatocytes. In some embodiments, the engineered tissue construct includes a population of hepatocytes in an amount of from about 8×10⁵ to about 3×10¹⁰ hepatocytes. In some embodiments, the engineered tissue construct includes a population of hepatocytes in an amount of from about 9×10⁵ to about 3×10¹⁰ hepatocytes. In some embodiments, the engineered tissue construct includes a population of hepatocytes in an amount of from about 1×10⁶ to about 3×10¹⁰ hepatocytes. In some embodiments, the engineered tissue construct includes a population of hepatocytes in an amount of from about 2×10⁶ to about 3×10¹⁰ hepatocytes. In some embodiments, the engineered tissue construct includes a population of hepatocytes in an amount of from about 3×10⁶ to about 3×10¹⁰, from about 4×10⁶ to about 3×10¹⁰ hepatocytes. In some embodiments, the engineered tissue construct includes a population of hepatocytes in an amount of from about 5×10⁶ to about 3×10¹⁰ hepatocytes. In some embodiments, the engineered tissue construct includes a population of hepatocytes in an amount of from about 6×10⁶ to about 3×10¹⁰ hepatocytes. In some embodiments, the engineered tissue construct includes a population of hepatocytes in an amount of from about 7×10⁶ to about 3×10¹⁰ hepatocytes. In some embodiments, the engineered tissue construct includes a population of hepatocytes in an amount of from about 8×10⁶ to about 3×10¹⁰ hepatocytes. In some embodiments, the engineered tissue construct includes a population of hepatocytes in an amount of from about 9×10⁶ to about 3×10¹⁰ hepatocytes. In some embodiments, the engineered tissue construct includes a population of hepatocytes in an amount of 1×10⁷ to about 3×10¹⁰ hepatocytes. In some embodiments, the engineered tissue construct includes a population of hepatocytes in an amount of from about 2×10⁷ to about 3×10¹⁰ hepatocytes. In some embodiments, the engineered tissue construct includes a population of hepatocytes in an amount of from about 3×10⁷ to about 3×10¹⁰ hepatocytes. In some embodiments, the engineered tissue construct includes a population of hepatocytes in an amount of from about 4×10⁷ to about 3×10¹⁰ hepatocytes. In some embodiments, the engineered tissue construct includes a population of hepatocytes in an amount of from about 5×10⁷ to about 3×10¹⁰ hepatocytes. In some embodiments, the engineered tissue construct includes a population of hepatocytes in an amount of from about 6×10⁷ to about 3×10¹⁰ hepatocytes. In some embodiments, the engineered tissue construct includes a population of hepatocytes in an amount of from about 7×10⁷ to about 3×10¹⁰ hepatocytes. In some embodiments, the engineered tissue construct includes a population of hepatocytes in an amount of from about 8×10⁷ to about 3×10¹⁰ hepatocytes. In some embodiments, the engineered tissue construct includes a population of hepatocytes in an amount of from about 9×10⁷ to about 3×10¹⁰ hepatocytes. In some embodiments, the engineered tissue construct includes a population of hepatocytes in an amount of from about 1×10⁸ to about 3×10¹⁰ hepatocytes. In some embodiments, the engineered tissue construct includes a population of hepatocytes in an amount of from about 2×10⁸ to about 3×10¹⁰ hepatocytes. In some embodiments, the engineered tissue construct includes a population of hepatocytes in an amount of from about 3×10⁸ to about 3×10¹⁰ hepatocytes. In some embodiments, the engineered tissue construct includes a population of hepatocytes in an amount of from about 4×10⁸ to about 3×10¹⁰ hepatocytes. In some embodiments, the engineered tissue construct includes a population of hepatocytes in an amount of from about 5×10⁸ to about 3×10¹⁰ hepatocytes. In some embodiments, the engineered tissue construct includes a population of hepatocytes in an amount of from about 6×10⁸ to about 3×10¹⁰ hepatocytes. In some embodiments, the engineered tissue construct includes a population of hepatocytes in an amount of from about 7×10⁸ to about 3×10¹⁰ hepatocytes. In some embodiments, the engineered tissue construct includes a population of hepatocytes in an amount of from about 8×10⁸ to about 3×10¹⁰ hepatocytes. In some embodiments, the engineered tissue construct includes a population of hepatocytes in an amount of from about 9×10⁸ to about 3×10¹⁰ hepatocytes. In some embodiments, the engineered tissue construct includes a population of hepatocytes in an amount of from about 1×10⁹ to about 3×10¹⁰ hepatocytes. In some embodiments, the engineered tissue construct includes a population of hepatocytes in an amount of from about 2×10⁹ to about 3×10¹⁰ hepatocytes. In some embodiments, the engineered tissue construct includes a population of hepatocytes in an amount of from about 3×10⁹ to about 3×10¹⁰ hepatocytes. In some embodiments, the engineered tissue construct includes a population of hepatocytes in an amount of from about 4×10⁹ to about 3×10¹⁰ hepatocytes. In some embodiments, the engineered tissue construct includes a population of hepatocytes in an amount of from about 5×10⁹ to about 3×10¹⁰ hepatocytes. In some embodiments, the engineered tissue construct includes a population of hepatocytes in an amount of from about 6×10⁹ to about 3×10¹⁰ hepatocytes. In some embodiments, the engineered tissue construct includes a population of hepatocytes in an amount of from about 7×10⁹ to about 3×10¹⁰ hepatocytes. In some embodiments, the engineered tissue construct includes a population of hepatocytes in an amount of from about 8×10⁹ to about 3×10¹⁰ hepatocytes. In some embodiments, the engineered tissue construct includes a population of hepatocytes in an amount of from about 9×10⁹ to about 3×10¹⁰ hepatocytes. In some embodiments, the engineered tissue construct includes a population of hepatocytes in an amount of from about 1×10¹⁰ to about 3×10¹⁰ hepatocytes. In some embodiments, the engineered tissue construct includes a population of hepatocytes in an amount of from about 2×10¹⁰ to about 3×10¹⁰ hepatocytes.

In some embodiments, the engineered tissue construct includes a population of hepatocytes in an amount of about 3×10⁵ hepatocytes. In some embodiments, the engineered tissue construct includes a population of hepatocytes in an amount of about 4×10⁵ hepatocytes. In some embodiments, the engineered tissue construct includes a population of hepatocytes in an amount of about 5×10⁵ hepatocytes. In some embodiments, the engineered tissue construct includes a population of hepatocytes in an amount of about 6×10⁵ hepatocytes. In some embodiments, the engineered tissue construct includes a population of hepatocytes in an amount of about 7×10⁵ hepatocytes. In some embodiments, the engineered tissue construct includes a population of hepatocytes in an amount of about 8×10⁵ hepatocytes. In some embodiments, the engineered tissue construct includes a population of hepatocytes in an amount of about 9×10⁵ hepatocytes. In some embodiments, the engineered tissue construct includes a population of hepatocytes in an amount of about 1×10⁶ hepatocytes. In some embodiments, the engineered tissue construct includes a population of hepatocytes in an amount of about 2×10⁶ hepatocytes. In some embodiments, the engineered tissue construct includes a population of hepatocytes in an amount of about 3×10⁶ hepatocytes. In some embodiments, the engineered tissue construct includes a population of hepatocytes in an amount of about 4×10⁶ hepatocytes. In some embodiments, the engineered tissue construct includes a population of hepatocytes in an amount of about 5×10⁶ hepatocytes. In some embodiments, the engineered tissue construct includes a population of hepatocytes in an amount of about 6×10⁶ hepatocytes. In some embodiments, the engineered tissue construct includes a population of hepatocytes in an amount of about 7×10⁶ hepatocytes. In some embodiments, the engineered tissue construct includes a population of hepatocytes in an amount of about 8×10⁶ hepatocytes. In some embodiments, the engineered tissue construct includes a population of hepatocytes in an amount of about 9×10⁶ hepatocytes. In some embodiments, the engineered tissue construct includes a population of hepatocytes in an amount of about 1×10⁷ hepatocytes. In some embodiments, the engineered tissue construct includes a population of hepatocytes in an amount of about 2×10⁷ hepatocytes. In some embodiments, the engineered tissue construct includes a population of hepatocytes in an amount of about 3×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes.

IIIaii. Infant (e.g., 1 Year of Age)

In some embodiments, the engineered tissue construct is implanted in an infant and includes a population of hepatocytes in an amount of from about 2×10⁷ to about 6×10¹⁰ (e.g., from about 3×10⁷ to about 6×10¹⁰, from about 4×10⁷ to about 6×10¹⁰, from about 5×10⁷ to about 6×10¹⁰, from about 6×10⁷ to about 6×10¹⁰, from about 7×10⁷ to about 6×10¹⁰, from about 8×10⁷ to about 6×10¹⁰, from about 9×10⁷ to about 6×10¹⁰, from about 1×10⁸ to about 6×10¹⁰, from about 2×10⁸ to about 6×10¹⁰, from about 3×10⁸ to about 6×10¹⁰, from about 4×10⁸ to about 6×10¹⁰, from about 5×10⁸ to about 6×10¹⁰, from about 6×10⁸ to about 6×10¹⁰, from about 7×10⁸ to about 6×10¹⁰, from about 8×10⁸ to about 6×10¹⁰, from about 9×10⁸ to about 6×10¹⁰, from about 1×10⁹ to about 6×10¹⁰, from about 2×10⁹ to about 6×10¹⁰, from about 3×10⁹ to about 6×10¹⁰, from about 4×10⁹ to about 6×10¹⁰, from about 5×10⁹ to about 6×10¹⁰, from about 6×10⁹ to about 6×10¹⁰, from about 7×10⁹ to about 6×10¹⁰, from about 8×10⁹ to about 6×10¹⁰, from about 9×10⁹ to about 6×10¹⁰, from about 1×10¹⁰ to about 6×10¹⁰, from about 2×10¹⁰ to about 6×10¹⁰, from about 3×10¹⁰ to about 6×10¹⁰, from about 4×10¹⁰ to about 6×10¹⁰, or from about 5×10¹⁰ to about 6×10¹⁰) hepatocytes. For example, in some embodiments, the engineered tissue construct includes a population of hepatocytes in an amount of from about 3×10⁷ to about 6×10¹⁰ hepatocytes. In some embodiments, the engineered tissue construct includes a population of hepatocytes in an amount of from about 4×10⁷ to about 6×10¹⁰ hepatocytes. In some embodiments, the engineered tissue construct includes a population of hepatocytes in an amount of from about 5×10⁷ to about 6×10¹⁰ hepatocytes. In some embodiments, the engineered tissue construct includes a population of hepatocytes in an amount of from about 6×10⁷ to about 6×10¹⁰ hepatocytes. In some embodiments, the engineered tissue construct includes a population of hepatocytes in an amount of from about 7×10⁷ to about 6×10¹⁰ hepatocytes. In some embodiments, the engineered tissue construct includes a population of hepatocytes in an amount of from about 8×10⁷ to about 6×10¹⁰ hepatocytes. In some embodiments, the engineered tissue construct includes a population of hepatocytes in an amount of from about 9×10⁷ to about 6×10¹⁰ hepatocytes. In some embodiments, the engineered tissue construct includes a population of hepatocytes in an amount of from about 1×10⁸ to about 6×10¹⁰ hepatocytes. In some embodiments, the engineered tissue construct includes a population of hepatocytes in an amount of from about 2×10⁸ to about 6×10¹⁰ hepatocytes. In some embodiments, the engineered tissue construct includes a population of hepatocytes in an amount of from about 3×10⁸ to about 6×10¹⁰ hepatocytes. In some embodiments, the engineered tissue construct includes a population of hepatocytes in an amount of from about 4×10⁸ to about 6×10¹⁰ hepatocytes. In some embodiments, the engineered tissue construct includes a population of hepatocytes in an amount of from about 5×10⁸ to about 6×10¹⁰ hepatocytes. In some embodiments, the engineered tissue construct includes a population of hepatocytes in an amount of from about 6×10⁸ to about 6×10¹⁰ hepatocytes. In some embodiments, the engineered tissue construct includes a population of hepatocytes in an amount of from about 7×10⁸ to about 6×10¹⁰ hepatocytes. In some embodiments, the engineered tissue construct includes a population of hepatocytes in an amount of from about 8×10⁸ to about 6×10¹⁰ hepatocytes. In some embodiments, the engineered tissue construct includes a population of hepatocytes in an amount of from about 9×10⁸ to about 6×10¹⁰ hepatocytes. In some embodiments, the engineered tissue construct includes a population of hepatocytes in an amount of from about 1×10⁹ to about 6×10¹⁰ hepatocytes. In some embodiments, the engineered tissue construct includes a population of hepatocytes in an amount of from about 2×10⁹ to about 6×10¹⁰ hepatocytes. In some embodiments, the engineered tissue construct includes a population of hepatocytes in an amount of from about 3×10⁹ to about 6×10¹⁰ hepatocytes. In some embodiments, the engineered tissue construct includes a population of hepatocytes in an amount of from about 4×10⁹ to about 6×10¹⁰ hepatocytes. In some embodiments, the engineered tissue construct includes a population of hepatocytes in an amount of from about 5×10⁹ to about 6×10¹⁰ hepatocytes. In some embodiments, the engineered tissue construct includes a population of hepatocytes in an amount of from about 6×10⁹ to about 6×10¹⁰ hepatocytes. In some embodiments, the engineered tissue construct includes a population of hepatocytes in an amount of from about 7×10⁹ to about 6×10¹⁰ hepatocytes. In some embodiments, the engineered tissue construct includes a population of hepatocytes in an amount of from about 8×10⁹ to about 6×10¹⁰ hepatocytes. In some embodiments, the engineered tissue construct includes a population of hepatocytes in an amount of from about 9×10⁹ to about 6×10¹⁰ hepatocytes. In some embodiments, the engineered tissue construct includes a population of hepatocytes in an amount of from about 1×10¹⁰ to about 6×10¹⁰ hepatocytes. In some embodiments, the engineered tissue construct includes a population of hepatocytes in an amount of from about 2×10¹⁰ to about 6×10¹⁰ hepatocytes. In some embodiments, the engineered tissue construct includes a population of hepatocytes in an amount of from about 3×10¹⁰ to about 6×10¹⁰ hepatocytes. In some embodiments, the engineered tissue construct includes a population of hepatocytes in an amount of from about 4×10¹⁰ to about 6×10¹⁰ hepatocytes. In some embodiments, the engineered tissue construct includes a population of hepatocytes in an amount of from about 5×10¹⁰ to about 6×10¹⁰ hepatocytes.

In some embodiments, the engineered tissue construct includes a population of hepatocytes in an amount of about 2×10⁷ hepatocytes. In some embodiments, the engineered tissue construct includes a population of hepatocytes in an amount of about 3×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes.

IIIaiii. Child (e.g., 5 Years of Age)

In some embodiments, the engineered tissue construct is implanted in a child and includes a population of hepatocytes in an amount of from about 3.5×10⁷ to about 1.05×10¹¹ (e.g., from about 4×10⁷ to about 1.05×10¹¹, from about 5×10⁷ to about 1.05×10¹¹, from about 6×10⁷ to about 1.05×10¹¹, from about 7×10⁷ to about 1.05×10¹¹, from about 8×10⁷ to about 1.05×10¹¹, from about 9×10⁷ to about 1.05×10¹¹, from about 1×10⁸ to about 1.05×10¹¹, from about 2×10⁸ to about 1.05×10¹¹, from about 3×10⁸ to about 1.05×10¹¹, from about 4×10⁸ to about 1.05×10¹¹, from about 5×10⁸ to about 1.05×10¹¹, from about 6×10⁸ to about 1.05×10¹¹, from about 7×10⁸ to about 1.05×10¹¹, from about 8×10⁸ to about 1.05×10¹¹, from about 9×10⁸ to about 1.05×10¹¹, from about 1×10⁹ to about 1.05×10¹¹, from about 2×10⁹ to about 1.05×10¹¹, from about 3×10⁹ to about 1.05×10¹¹, from about 4×10⁹ to about 1.05×10¹¹, from about 5×10⁹ to about 1.05×10¹¹, from about 6×10⁹ to about 1.05×10¹¹, from about 7×10⁹ to about 1.05×10¹¹, from about 8×10⁹ to about 1.05×10¹¹, from about 9×10⁹ to about 1.05×10¹¹, from about 1×10¹⁰ to about 1.05×10¹¹, from about 2×10¹⁰ to about 1.05×10¹¹, from about 3×10¹⁰ to about 1.05×10¹¹, from about 4×10¹⁰ to about 1.05×10¹¹, from about 5×10¹⁰ to about 1.05×10¹¹, from about 6×10¹⁰ to about 1.05×10¹¹, from about 7×10¹⁰ to about 1.05×10¹¹, from about 8×10¹⁰ to about 1.05×10¹¹, from about 9×10¹⁰ to about 1.05×10¹¹, from about 1×10¹¹ to about 1.05×10¹¹) hepatocytes. For example, in some embodiments, the engineered tissue construct includes a population of hepatocytes in an amount of from about 4×10⁷ to about 1.05×10¹¹ hepatocytes. In some embodiments, the engineered tissue construct includes a population of hepatocytes in an amount of from about 5×10⁷ to about 1.05×10¹¹ hepatocytes. In some embodiments, the engineered tissue construct includes a population of hepatocytes in an amount of from about 6×10⁷ to about 1.05×10¹¹ hepatocytes.

In some embodiments, the engineered tissue construct includes a population of hepatocytes in an amount of from about 7×10⁷ to about 1.05×10¹¹. In some embodiments, the engineered tissue construct includes a population of hepatocytes in an amount of from about 8×10⁷ to about 1.05×10¹¹ hepatocytes. In some embodiments, the engineered tissue construct includes a population of hepatocytes in an amount of from about 9×10⁷ to about 1.05×10¹¹ hepatocytes. In some embodiments, the engineered tissue construct includes a population of hepatocytes in an amount of from about 1×10⁸ to about 1.05×10¹¹ hepatocytes. In some embodiments, the engineered tissue construct includes a population of hepatocytes in an amount of from about 2×10⁸ to about 1.05×10¹¹ hepatocytes. In some embodiments, the engineered tissue construct includes a population of hepatocytes in an amount of from about 3×10⁸ to about 1.05×10¹¹ hepatocytes. In some embodiments, the engineered tissue construct includes a population of hepatocytes in an amount of from about 4×10⁸ to about 1.05×10¹¹ hepatocytes. In some embodiments, the engineered tissue construct includes a population of hepatocytes in an amount of from about 5×10⁸ to about 1.05×10¹¹ hepatocytes. In some embodiments, the engineered tissue construct includes a population of hepatocytes in an amount of from about 6×10⁸ to about 1.05×10¹¹ hepatocytes. In some embodiments, the engineered tissue construct includes a population of hepatocytes in an amount of from about 7×10⁸ to about 1.05×10¹¹. In some embodiments, the engineered tissue construct includes a population of hepatocytes in an amount of from about 8×10⁸ to about 1.05×10¹¹ hepatocytes. In some embodiments, the engineered tissue construct includes a population of hepatocytes in an amount of from about 9×10⁸ to about 1.05×10¹¹ hepatocytes. In some embodiments, the engineered tissue construct includes a population of hepatocytes in an amount of from about 1×10⁹ to about 1.05×10¹¹ hepatocytes. In some embodiments, the engineered tissue construct includes a population of hepatocytes in an amount of from about 2×10⁹ to about 1.05×10¹¹ hepatocytes. In some embodiments, the engineered tissue construct includes a population of hepatocytes in an amount of from about 3×10⁹ to about 1.05×10¹¹ hepatocytes. In some embodiments, the engineered tissue construct includes a population of hepatocytes in an amount of from about 4×10⁹ to about 1.05×10¹¹ hepatocytes. In some embodiments, the engineered tissue construct includes a population of hepatocytes in an amount of from about 5×10⁹ to about 1.05×10¹¹ hepatocytes. In some embodiments, the engineered tissue construct includes a population of hepatocytes in an amount of from about 6×10⁹ to about 1.05×10¹¹ hepatocytes. In some embodiments, the engineered tissue construct includes a population of hepatocytes in an amount of from about 7×10⁹ to about 1.05×10¹¹. In some embodiments, the engineered tissue construct includes a population of hepatocytes in an amount of from about 8×10⁹ to about 1.05×10¹¹ hepatocytes. In some embodiments, the engineered tissue construct includes a population of hepatocytes in an amount of from about 9×10⁹ to about 1.05×10¹¹ hepatocytes. In some embodiments, the engineered tissue construct includes a population of hepatocytes in an amount of from about 1×10¹⁰ to about 1.05×10¹¹ hepatocytes. In some embodiments, the engineered tissue construct includes a population of hepatocytes in an amount of from about 2×10¹⁰ to about 1.05×10¹¹ hepatocytes. In some embodiments, the engineered tissue construct includes a population of hepatocytes in an amount of from about 3×10¹⁰ to about 1.05×10¹¹ hepatocytes. In some embodiments, the engineered tissue construct includes a population of hepatocytes in an amount of from about 4×10¹⁰ to about 1.05×10¹¹ hepatocytes. In some embodiments, the engineered tissue construct includes a population of hepatocytes in an amount of from about 5×10¹⁰ to about 1.05×10¹¹ hepatocytes. In some embodiments, the engineered tissue construct includes a population of hepatocytes in an amount of from about 6×10¹⁰ to about 1.05×10¹¹ hepatocytes. In some embodiments, the engineered tissue construct includes a population of hepatocytes in an amount of from about 7×10¹⁰ to about 1.05×10¹¹. In some embodiments, the engineered tissue construct includes a population of hepatocytes in an amount of from about 8×10¹⁰ to about 1.05×10¹¹ hepatocytes. In some embodiments, the engineered tissue construct includes a population of hepatocytes in an amount of from about 9×10¹⁰ to about 1.05×10¹¹ hepatocytes. In some embodiments, the engineered tissue construct includes a population of hepatocytes in an amount of from about 1×10¹¹ to about 1.05×10¹¹ hepatocytes.

In some embodiments, the engineered tissue construct includes a population of hepatocytes in an amount of about 3.5×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.05×10¹¹ hepatocytes.

IIIaiv. Child (e.g., 9 Years of Age)

In some embodiments, the engineered tissue construct is implanted in a child and includes a population of hepatocytes in an amount of from about 4.5×10⁷ to about 1.35×10¹¹ (e.g., from about 5×10⁷ to about 1.35×10¹¹, from about 6×10⁷ to about 1.35×10¹¹, from about 7×10⁷ to about 1.35×10¹¹, from about 8×10⁸ to about 1.35×10¹¹, from about 9×10⁸ to about 1.35×10¹¹, from about 1×10⁸ to about 1.35×10¹¹, from about 2×10⁸ to about 1.35×10¹¹, from about 3×10⁸ to about 1.35×10¹¹, from about 4×10⁸ to about 1.35×10¹¹, from about 5×10⁸ to about 1.35×10¹¹, from about 6×10⁸ to about 1.35×10¹¹, from about 7×10⁸ to about 1.35×10¹¹, from about 8×10⁸ to about 1.35×10¹¹, from about 9×10⁸ to about 1.35×10¹¹, from about 1×10⁹ to about 1.35×10¹¹, from about 2×10⁹ to about 1.35×10¹¹, from about 3×10⁹ to about 1.35×10¹¹, from about 4×10⁹ to about 1.35×10¹¹, from about 5×10⁹ to about 1.35×10¹¹, from about 6×10⁹ to about 1.35×10¹¹, from about 7×10⁹ to about 1.35×10¹¹, from about 8×10⁹ to about 1.35×10¹¹, from about 9×10⁹ to about 1.35×10¹¹, from about 1×10¹⁰ to about 1.35×10¹¹, from about 2×10¹⁰ to about 1.35×10¹¹, from about 3×10¹⁰ to about 1.35×10¹¹, from about 4×10¹⁰ to about 1.35×10¹¹, from about 5×10¹⁰ to about 1.35×10¹¹, from about 6×10¹⁰ to about 1.35×10¹¹, from about 7×10¹⁰ to about 1.35×10¹¹, from about 8×10¹⁰ to about 1.35×10¹¹, from about 9×10¹⁰ to about 1.35×10¹¹, from about 1×10¹¹ to about 1.35×10¹¹) hepatocytes. For example, in some embodiments, the engineered tissue construct includes a population of hepatocytes in an amount of from about 4×10⁷ to about 1.35×10¹¹ hepatocytes. In some embodiments, the engineered tissue construct includes a population of hepatocytes in an amount of from about 5×10⁷ to about 1.35×10¹¹ hepatocytes. In some embodiments, the engineered tissue construct includes a population of hepatocytes in an amount of from about 6×10⁷ to about 1.35×10¹¹ hepatocytes. In some embodiments, the engineered tissue construct includes a population of hepatocytes in an amount of from about 7×10⁷ to about 1.35×10¹¹. In some embodiments, the engineered tissue construct includes a population of hepatocytes in an amount of from about 8×10⁷ to about 1.35×10¹¹ hepatocytes. In some embodiments, the engineered tissue construct includes a population of hepatocytes in an amount of from about 9×10⁷ to about 1.35×10¹¹ hepatocytes. In some embodiments, the engineered tissue construct includes a population of hepatocytes in an amount of from about 1×10⁸ to about 1.35×10¹¹ hepatocytes. In some embodiments, the engineered tissue construct includes a population of hepatocytes in an amount of from about 2×10⁸ to about 1.35×10¹¹ hepatocytes. In some embodiments, the engineered tissue construct includes a population of hepatocytes in an amount of from about 3×10⁸ to about 1.35×10¹¹ hepatocytes. In some embodiments, the engineered tissue construct includes a population of hepatocytes in an amount of from about 4×10⁸ to about 1.35×10¹¹ hepatocytes. In some embodiments, the engineered tissue construct includes a population of hepatocytes in an amount of from about 5×10⁸ to about 1.35×10¹¹ hepatocytes. In some embodiments, the engineered tissue construct includes a population of hepatocytes in an amount of from about 6×10⁸ to about 1.35×10¹¹ hepatocytes. In some embodiments, the engineered tissue construct includes a population of hepatocytes in an amount of from about 7×10⁸ to about 1.35×10¹¹. In some embodiments, the engineered tissue construct includes a population of hepatocytes in an amount of from about 8×10⁸ to about 1.35×10¹¹ hepatocytes. In some embodiments, the engineered tissue construct includes a population of hepatocytes in an amount of from about 9×10⁸ to about 1.35×10¹¹ hepatocytes. In some embodiments, the engineered tissue construct includes a population of hepatocytes in an amount of from about 1×10⁹ to about 1.35×10¹¹ hepatocytes. In some embodiments, the engineered tissue construct includes a population of hepatocytes in an amount of from about 2×10⁹ to about 1.35×10¹¹ hepatocytes. In some embodiments, the engineered tissue construct includes a population of hepatocytes in an amount of from about 3×10⁹ to about 1.35×10¹¹ hepatocytes. In some embodiments, the engineered tissue construct includes a population of hepatocytes in an amount of from about 4×10⁹ to about 1.35×10¹¹ hepatocytes. In some embodiments, the engineered tissue construct includes a population of hepatocytes in an amount of from about 5×10⁹ to about 1.35×10¹¹ hepatocytes. In some embodiments, the engineered tissue construct includes a population of hepatocytes in an amount of from about 6×10⁹ to about 1.35×10¹¹ hepatocytes. In some embodiments, the engineered tissue construct includes a population of hepatocytes in an amount of from about 7×10⁹ to about 1.35×10¹¹. In some embodiments, the engineered tissue construct includes a population of hepatocytes in an amount of from about 8×10⁹ to about 1.35×10¹¹ hepatocytes. In some embodiments, the engineered tissue construct includes a population of hepatocytes in an amount of from about 9×10⁹ to about 1.35×10¹¹ hepatocytes. In some embodiments, the engineered tissue construct includes a population of hepatocytes in an amount of from about 1×10¹⁰ to about 1.35×10¹¹ hepatocytes. In some embodiments, the engineered tissue construct includes a population of hepatocytes in an amount of from about 2×10¹⁰ to about 1.35×10¹¹ hepatocytes. In some embodiments, the engineered tissue construct includes a population of hepatocytes in an amount of from about 3×10¹⁰ to about 1.35×10¹¹ hepatocytes. In some embodiments, the engineered tissue construct includes a population of hepatocytes in an amount of from about 4×10¹⁰ to about 1.35×10¹¹ hepatocytes. In some embodiments, the engineered tissue construct includes a population of hepatocytes in an amount of from about 5×10¹⁰ to about 1.35×10¹¹ hepatocytes. In some embodiments, the engineered tissue construct includes a population of hepatocytes in an amount of from about 6×10¹⁰ to about 1.35×10¹¹ hepatocytes. In some embodiments, the engineered tissue construct includes a population of hepatocytes in an amount of from about 7×10¹⁰ to about 1.35×10¹¹. In some embodiments, the engineered tissue construct includes a population of hepatocytes in an amount of from about 8×10¹⁰ to about 1.35×10¹¹ hepatocytes. In some embodiments, the engineered tissue construct includes a population of hepatocytes in an amount of from about 9×10¹⁰ to about 1.35×10¹¹ hepatocytes. In some embodiments, the engineered tissue construct includes a population of hepatocytes in an amount of from about 1×10¹¹ to about 1.35×10¹¹ hepatocytes.

In some embodiments, the engineered tissue construct includes a population of hepatocytes in an amount of about 3.5×10⁷ hepatocytes. In some embodiments, the engineered tissue construct includes a population of hepatocytes in an amount of about 4×10⁷ hepatocytes. In some embodiments, the engineered tissue construct includes a population of hepatocytes in an amount of about 5×10⁷ hepatocytes. In some embodiments, the engineered tissue construct includes a population of hepatocytes in an amount of about 6×10⁷ hepatocytes. In some embodiments, the engineered tissue construct includes a population of hepatocytes in an amount of about 7×10⁷ hepatocytes. In some embodiments, the engineered tissue construct includes a population of hepatocytes in an amount of about 8×10⁷ hepatocytes. In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.35×10¹¹ hepatocytes.

IIIav. Adult (e.g., 18 Years of Age and Older)

In some embodiments, the engineered tissue construct is implanted in an adult and includes a population of hepatocytes in an amount of from about 9×10⁷ to about 1.8×10¹¹ (e.g., from about 1×10⁸ to about 1.8×10¹¹, from about 2×10⁸ to about 1.8×10¹¹, from about 3×10⁸ to about 1.8×10¹¹, from about 4×10⁸ to about 1.8×10¹¹, from about 5×10⁸ to about 1.8×10¹¹, from about 6×10⁸ to about 1.8×10¹¹, from about 7×10⁸ to about 1.8×10¹¹, from about 8×10⁸ to about 1.8×10¹¹, from about 9×10⁸ to about 1.8×10¹¹, from about 1×10⁹ to about 1.8×10¹¹, from about 2×10⁹ to about 1.8×10¹¹, from about 3×10⁹ to about 1.8×10¹¹, from about 4×10⁹ to about 1.8×10¹¹, from about 5×10⁹ to about 1.8×10¹¹, from about 6×10⁹ to about 1.8×10¹¹, from about 7×10⁹ to about 1.8×10¹¹, from about 8×1 20 to about 1.8×10¹¹, from about 9×10⁹ to about 1.8×10¹¹, from about 1×10¹⁰ to about 1.8×10¹¹, from about 2×10¹⁰ to about 1.8×10¹¹, from about 3×10¹⁰ to about 1.8×10¹¹, from about 4×10¹⁰ to about 1.8×10¹¹, from about 5×10¹⁰ to about 1.8×10¹¹, from about 6×10¹⁰ to about 1.8×10¹¹, from about 7×10¹⁰ to about 1.8×10¹¹, from about 8×10¹⁰ to about 1.8×10¹¹, from about 9×10¹⁰ to about 1.8×10¹¹, or from about 1×10¹¹ to about 1.8×10¹¹) hepatocytes. For example, in some embodiments, the engineered tissue construct includes a population of hepatocytes in an amount of from about 4×10⁷ to about 1.8×10¹¹ hepatocytes. In some embodiments, the engineered tissue construct includes a population of hepatocytes in an amount of from about 5×10⁷ to about 1.8×10¹¹ hepatocytes. In some embodiments, the engineered tissue construct includes a population of hepatocytes in an amount of from about 6×10⁷ to about 1.8×10¹¹ hepatocytes. In some embodiments, the engineered tissue construct includes a population of hepatocytes in an amount of from about 7×10⁷ to about 1.8×10¹¹. In some embodiments, the engineered tissue construct includes a population of hepatocytes in an amount of from about 8×10⁷ to about 1.8×10¹¹ hepatocytes. In some embodiments, the engineered tissue construct includes a population of hepatocytes in an amount of from about 9×10⁷ to about 1.8×10¹¹ hepatocytes. In some embodiments, the engineered tissue construct includes a population of hepatocytes in an amount of from about 1×10⁸ to about 1.8×10¹¹ hepatocytes. In some embodiments, the engineered tissue construct includes a population of hepatocytes in an amount of from about 2×10⁸ to about 1.8×10¹¹ hepatocytes. In some embodiments, the engineered tissue construct includes a population of hepatocytes in an amount of from about 3×10⁸ to about 1.8×10¹¹ hepatocytes. In some embodiments, the engineered tissue construct includes a population of hepatocytes in an amount of from about 4×10⁸ to about 1.8×10¹¹ hepatocytes. In some embodiments, the engineered tissue construct includes a population of hepatocytes in an amount of from about 5×10⁸ to about 1.8×10¹¹ hepatocytes. In some embodiments, the engineered tissue construct includes a population of hepatocytes in an amount of from about 6×10⁸ to about 1.8×10¹¹ hepatocytes. In some embodiments, the engineered tissue construct includes a population of hepatocytes in an amount of from about 7×10⁸ to about 1.8×10¹¹. In some embodiments, the engineered tissue construct includes a population of hepatocytes in an amount of from about 8×10⁸ to about 1.8×10¹¹ hepatocytes. In some embodiments, the engineered tissue construct includes a population of hepatocytes in an amount of from about 9×10⁸ to about 1.8×10¹¹ hepatocytes. In some embodiments, the engineered tissue construct includes a population of hepatocytes in an amount of from about 1×10⁹ to about 1.8×10¹¹ hepatocytes. In some embodiments, the engineered tissue construct includes a population of hepatocytes in an amount of from about 2×10⁹ to about 1.8×10¹¹ hepatocytes. In some embodiments, the engineered tissue construct includes a population of hepatocytes in an amount of from about 3×10⁹ to about 1.8×10¹¹ hepatocytes. In some embodiments, the engineered tissue construct includes a population of hepatocytes in an amount of from about 4×10⁹ to about 1.8×10¹¹ hepatocytes. In some embodiments, the engineered tissue construct includes a population of hepatocytes in an amount of from about 5×10⁹ to about 1.8×10¹¹ hepatocytes. In some embodiments, the engineered tissue construct includes a population of hepatocytes in an amount of from about 6×10⁹ to about 1.8×10¹¹ hepatocytes. In some embodiments, the engineered tissue construct includes a population of hepatocytes in an amount of from about 7×10⁹ to about 1.8×10¹¹. In some embodiments, the engineered tissue construct includes a population of hepatocytes in an amount of from about 8×10⁹ to about 1.8×10¹¹ hepatocytes. In some embodiments, the engineered tissue construct includes a population of hepatocytes in an amount of from about 9×10⁹ to about 1.8×10¹¹ hepatocytes. In some embodiments, the engineered tissue construct includes a population of hepatocytes in an amount of from about 1×10¹⁰ to about 1.8×10¹¹ hepatocytes. In some embodiments, the engineered tissue construct includes a population of hepatocytes in an amount of from about 2×10¹⁰ to about 1.8×10¹¹ hepatocytes. In some embodiments, the engineered tissue construct includes a population of hepatocytes in an amount of from about 3×10¹⁰ to about 1.8×10¹¹ hepatocytes. In some embodiments, the engineered tissue construct includes a population of hepatocytes in an amount of from about 4×10¹⁰ to about 1.8×10¹¹ hepatocytes. In some embodiments, the engineered tissue construct includes a population of hepatocytes in an amount of from about 5×10¹⁰ to about 1.8×10¹¹ hepatocytes. In some embodiments, the engineered tissue construct includes a population of hepatocytes in an amount of from about 6×10¹⁰ to about 1.8×10¹¹ hepatocytes. In some embodiments, the engineered tissue construct includes a population of hepatocytes in an amount of from about 7×10¹⁰ to about 1.8×10¹¹. In some embodiments, the engineered tissue construct includes a population of hepatocytes in an amount of from about 8×10¹⁰ to about 1.8×10¹¹ hepatocytes. In some embodiments, the engineered tissue construct includes a population of hepatocytes in an amount of from about 9×10¹⁰ to about 1.8×10¹¹ hepatocytes. In some embodiments, the engineered tissue construct includes a population of hepatocytes in an amount of from about 1×10¹¹ to about 1.8×10¹¹ hepatocytes.

In some embodiments, the engineered tissue construct includes a population of hepatocytes in an amount of about 9×10⁷ hepatocytes. In some embodiments, the engineered tissue construct includes a population of hepatocytes in an amount of about 1×10⁸ hepatocytes. In some embodiments, the engineered tissue construct includes a population of hepatocytes in an amount of about 2×10⁸ hepatocytes. In some embodiments, the engineered tissue construct includes a population of hepatocytes in an amount of about 3×10⁸ hepatocytes. In some embodiments, the engineered tissue construct includes a population of hepatocytes in an amount of about 4×10⁸ hepatocytes. In some embodiments, the engineered tissue construct includes a population of hepatocytes in an amount of about 5×10⁸ hepatocytes. In some embodiments, the engineered tissue construct includes a population of hepatocytes in an amount of about 6×10⁸ hepatocytes. In some embodiments, the engineered tissue construct includes a population of hepatocytes in an amount of about 7×10⁸ hepatocytes. In some embodiments, the engineered tissue construct includes a population of hepatocytes in an amount of about 8×10⁸ hepatocytes. In some embodiments, the engineered tissue construct includes a population of hepatocytes in an amount of about 9×10⁸ hepatocytes. In some embodiments, the engineered tissue construct includes a population of hepatocytes in an amount of about 1×10⁹ hepatocytes. In some embodiments, the engineered tissue construct includes a population of hepatocytes in an amount of about 2×10⁹ hepatocytes. In some embodiments, the engineered tissue construct includes a population of hepatocytes in an amount of about 3×10⁹ hepatocytes. In some embodiments, the engineered tissue construct includes a population of hepatocytes in an amount of about 4×10⁹ hepatocytes. In some embodiments, the engineered tissue construct includes a population of hepatocytes in an amount of about 5×10⁹ hepatocytes. In some embodiments, the engineered tissue construct includes a population of hepatocytes in an amount of about 6×10⁹ hepatocytes. In some embodiments, the engineered tissue construct includes a population of hepatocytes in an amount of about 7×10⁹ hepatocytes. In some embodiments, the engineered tissue construct includes a population of hepatocytes in an amount of about 8×10⁹ hepatocytes. In some embodiments, the engineered tissue construct includes a population of hepatocytes in an amount of about 9×10⁹ hepatocytes. In some embodiments, the engineered tissue construct includes a population of hepatocytes in an amount of about 1×10¹⁰ hepatocytes. In some embodiments, the engineered tissue construct includes a population of hepatocytes in an amount of about 2×10¹⁰ hepatocytes. In some embodiments, the engineered tissue construct includes a population of hepatocytes in an amount of about 3×10¹⁰ hepatocytes. In some embodiments, the engineered tissue construct includes a population of hepatocytes in an amount of about 4×10¹⁰ hepatocytes. In some embodiments, the engineered tissue construct includes a population of hepatocytes in an amount of about 5×10¹⁰ hepatocytes. In some embodiments, the engineered tissue construct includes a population of hepatocytes in an amount of about 6×10¹⁰ hepatocytes. In some embodiments, the engineered tissue construct includes a population of hepatocytes in an amount of about 7×10¹⁰ hepatocytes. In some embodiments, the engineered tissue construct includes a population of hepatocytes in an amount of about 8×10¹⁰ hepatocytes. In some embodiments, the engineered tissue construct includes a population of hepatocytes in an amount of about 9×10¹⁰ hepatocytes. In some embodiments, the engineered tissue construct includes a population of hepatocytes in an amount of about 1×10¹¹ hepatocytes. In some embodiments, the engineered tissue construct includes a population of hepatocytes in an amount of about 1.8×10¹¹ hepatocytes.

### Stromal Cells

The engineered tissue constructs described herein optionally include stromal cells (e.g., fibroblasts). In some embodiments, the stromal cells are fibroblasts. In some embodiments, the fibroblasts are human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts, human vas deferens fibroblasts). In some embodiments, the fibroblasts are human dermal fibroblasts. In some embodiments, the fibroblasts are normal human dermal fibroblasts. In some embodiments, the fibroblasts are neonatal foreskin fibroblasts. In some embodiments, the fibroblasts human lung fibroblasts. In some embodiments, the fibroblasts are human ventricular cardiac fibroblasts. In some embodiments, the fibroblasts are human atrial cardiac fibroblasts. In some embodiments, the fibroblasts are human uterine fibroblasts. In some embodiments, the fibroblasts are human bladder fibroblasts. In some embodiments, the fibroblasts are human gingival fibroblasts. In some embodiments, the fibroblasts are human pericardial fibroblasts. In some embodiments, the fibroblasts are human gall bladder fibroblasts. In some embodiments, the fibroblasts are human portal vein fibroblasts. In some embodiments, the fibroblasts are vas deferens fibroblasts.

In some embodiments the engineered tissue construct includes an optional population of stromal cells (e.g., fibroblasts) in an amount that is effective to treat hyperammonemia in a subject.

In some embodiments the engineered tissue construct includes an optional population of stromal cells (e.g., fibroblasts) in an amount that is effective to reduce ammonia levels in the subject.

In some embodiments, the population of stromal cells (e.g., fibroblasts) is up to 1.8×10¹² (e.g., from about 1 to about 1.8×10¹², from about 10 to about 1.8×10¹², from about 100 to about 1.8×10¹², from about 1×10³ to about 1.8×10¹², from about 2×10³ to about 1.8×10¹², from about 3×10³ to about 1.8×10¹², from about 4×10³ to about 1.8×10¹², from about 5×10³ to about 1.8×10¹², from about 6×10³ to about 1.8×10¹², from about 7×10³ to about 1.8×10¹², from about 8×10³ to about 1.8×10¹², from about 9×10³ to about 1.8×10¹², from about 1×10⁴ to about 1.8×10¹², from about 2×10⁴ to about 1.8×10¹², from about 3×10⁴ to about 1.8×10¹², from about 4×10⁴ to about 1.8×10¹², from about 5×10⁴ to about 1.8×10¹², from about 6×10⁴ to about 1.8×10¹², from about 7×10⁴ to about 1.8×10¹², from about 8×10⁴ to about 1.8×10¹², from about 9×10⁴ to about 1.8×10¹², from about 1×10⁵ to about 1.8×10¹², from about 2×10⁵ to about 1.8×10¹², from about 3×10⁵ to about 1.8×10¹², from about 4×10⁵ to about 1.8×10¹², from about 5×10⁵ to about 1.8×10¹², from about 6×10⁵ to about 1.8×10¹², from about 7×10⁵ to about 1.8×10¹², from about 8×10⁵ to about 1.8×10¹², from about 9×10⁵ to about 1.8×10¹², from about 1×10⁶ to about 1.8×10¹², from about 2×10⁶ to about 1.8×10¹², 3×10⁶ to about 1.8×10¹², 4×10⁶ to about 1.8×10¹², 5×10⁶ to about 1.8×10¹², 6×10⁶ to about 1.8×10¹², 7×10⁶ to about 1.8×10¹², 8×10⁶ to about 1.8×10¹², 9×10⁶ to about 1.8×10¹², from about 1×10⁷ to about 1.8×10¹², from about 2×10⁷ to about 1.8×10¹², from about 3×10⁷ to about 1.8×10¹², from about 4×10⁷ to about 1.8×10¹², from about 5×10⁷ to about 1.8×10¹², from about 6×10⁷ to about 1.8×10¹², from about 7×10⁷ to about 1.8×10¹², from about 8×10⁷ to about 1.8×10¹², from about 9×10⁷ to about 1.8×10¹², from about 1×10⁸ to about 1.8×10¹², from about 2×10⁸ to about 1.8×10¹², from about 3×10⁸ to about 1.8×10¹², from about 4×10⁸ to about 1.8×10¹², from about 5×10⁸ to about 1.8×10¹², from about 6×10⁸ to about 1.8×10¹², from about 7×10⁸ to about 1.8×10¹², from about 8×10⁸ to about 1.8×10¹², from about 9×10⁸ to about 1.8×10¹², from about 1×10⁹ to about 1.8×10¹², from about 2×10⁹ to about 1.8×10¹², from about 3×10⁹ to about 1.8×10¹², from about 4×10⁹ to about 1.8×10¹², from about 5×10⁹ to about 1.8×10¹², from about 6×10⁹ to about 1.8×10¹², from about 7×10⁹ to about 1.8×10¹², from about 8×10⁹ to about 1.8×10¹², from about 9×10⁹ to about 1.8×10¹², from about 1×10¹⁰ to about 1.8×10¹², from about 2×10¹⁰ to about 1.8×10¹², from about 3×10¹⁰ to about 1.8×10¹², from about 4×10¹⁰ to about 1.8×10¹², from about 5×10¹⁰ to about 1.8×10¹², from about 6×10¹⁰ to about 1.8×10¹², from about 7×10¹⁰ to about 1.8×10¹², from about 8×10¹⁰ to about 1.8×10¹², from about 9×10¹⁰ to about 1.8×10¹², from about 1×10¹¹ to about 1.8×10¹², from about 2×10¹¹ to about 1.8×10¹², from about 3×10¹¹ to about 1.8×10¹², from about 4×10¹¹ to about 1.8×10¹², from about 5×10¹¹ to about 1.8×10¹², from about 6×10¹¹ to about 1.8×10¹², from about 7×10¹¹ to about 1.8×10¹², from about 8×10¹¹ to about 1.8×10¹², from about 9×10¹¹ to about 1.8×10¹², or from about 1×10¹² to about 1.8×10¹²) stromal cells (e.g., fibroblasts). For example, in some embodiments, the engineered tissue construct includes a population of stromal cells (e.g., fibroblasts) in an amount of from about 1 to about 1.8×10¹² stromal cells (e.g., fibroblasts). In some embodiments, the engineered tissue construct includes a population of stromal cells (e.g., fibroblasts) in an amount of from about 10 to about 1.8×10¹² stromal cells (e.g., fibroblasts). In some embodiments, the engineered tissue construct includes a population of stromal cells (e.g., fibroblasts) in an amount of from about 100 to about 1.8×10¹² stromal cells (e.g., fibroblasts). In some embodiments, the engineered tissue construct includes a population of stromal cells (e.g., fibroblasts) in an amount of from about 1×10³ to about 1.8×10¹² stromal cells (e.g., fibroblasts). In some embodiments, the engineered tissue construct includes a population of stromal cells (e.g., fibroblasts) in an amount from about 2×10³ to about 1.8×10¹² stromal cells (e.g., fibroblasts). In some embodiments, the engineered tissue construct includes a population of stromal cells (e.g., fibroblasts) in an amount of from about 3×10³ to about 1.8×10¹² stromal cells (e.g., fibroblasts). In some embodiments, the engineered tissue construct includes a population of stromal cells (e.g., fibroblasts) in an amount of from about 4×10³ to about 1.8×10¹² stromal cells (e.g., fibroblasts). In some embodiments, the engineered tissue construct includes a population of stromal cells (e.g., fibroblasts) in an amount of from about 5×10³ to about 1.8×10¹² stromal cells (e.g., fibroblasts). In some embodiments, the engineered tissue construct includes a population of stromal cells (e.g., fibroblasts) in an amount of from about 6×10³ to about 1.8×10¹² stromal cells (e.g., fibroblasts). In some embodiments, the engineered tissue construct includes a population of stromal cells (e.g., fibroblasts) in an amount of from about 7×10³ to about 1.8×10¹² stromal cells (e.g., fibroblasts). In some embodiments, the engineered tissue construct includes a population of stromal cells (e.g., fibroblasts) in an amount of from about 8×10³ to about 1.8×10¹² stromal cells (e.g., fibroblasts). In some embodiments, the engineered tissue construct includes a population of stromal cells (e.g., fibroblasts) in an amount of from about 9×10³ to about 1.8×10¹² stromal cells (e.g., fibroblasts). In some embodiments, the engineered tissue construct includes a population of stromal cells (e.g., fibroblasts) in an amount of from about 1×10⁴ to about 1.8×10¹² stromal cells (e.g., fibroblasts). In some embodiments, the engineered tissue construct includes a population of stromal cells (e.g., fibroblasts) in an amount of from about 2×10⁴ to about 1.8×10¹² stromal cells (e.g., fibroblasts). In some embodiments, the engineered tissue construct includes a population of stromal cells (e.g., fibroblasts) in an amount of from about 3×10⁴ to about 1.8×10¹² stromal cells (e.g., fibroblasts). In some embodiments, the engineered tissue construct includes a population of stromal cells (e.g., fibroblasts) in an amount of from about 4×10⁴ to about 1.8×10¹² stromal cells (e.g., fibroblasts). In some embodiments, the engineered tissue construct includes a population of stromal cells (e.g., fibroblasts) in an amount of from about 5×10⁴ to about 1.8×10¹² stromal cells (e.g., fibroblasts). In some embodiments, the engineered tissue construct includes a population of stromal cells (e.g., fibroblasts) in an amount of from about 6×10⁴ to about 1.8×10¹² stromal cells (e.g., fibroblasts). In some embodiments, the engineered tissue construct includes a population of stromal cells (e.g., fibroblasts) in an amount of from about 7×10⁴ to about 1.8×10¹² stromal cells (e.g., fibroblasts). In some embodiments, the engineered tissue construct includes a population of stromal cells (e.g., fibroblasts) in an amount of from about 8×10⁴ to about 1.8×10¹² stromal cells (e.g., fibroblasts). In some embodiments, the engineered tissue construct includes a population of stromal cells (e.g., fibroblasts) in an amount of from about 9×10⁴ to about 1.8×10¹² stromal cells (e.g., fibroblasts). In some embodiments, the engineered tissue construct includes a population of stromal cells (e.g., fibroblasts) in an amount of from about 1×10⁵ to about 1.8×10¹² stromal cells (e.g., fibroblasts). In some embodiments, the engineered tissue construct includes a population of stromal cells (e.g., fibroblasts) in an amount of from about 2×10⁵ to about 1.8×10¹² stromal cells (e.g., fibroblasts). In some embodiments, the engineered tissue construct includes a population of stromal cells (e.g., fibroblasts) in an amount of from about 3×10⁵ to about 1.8×10¹² stromal cells (e.g., fibroblasts). In some embodiments, the engineered tissue construct includes a population of stromal cells (e.g., fibroblasts) in an amount of from about 4×10⁵ to about 1.8×10¹² stromal cells (e.g., fibroblasts). In some embodiments, the engineered tissue construct includes a population of stromal cells (e.g., fibroblasts) in an amount of from about 5×10⁵ to about 1.8×10¹² stromal cells (e.g., fibroblasts). In some embodiments, the engineered tissue construct includes a population of stromal cells (e.g., fibroblasts) in an amount of from about 6×10⁵ to about 1.8×10¹² stromal cells (e.g., fibroblasts). In some embodiments, the engineered tissue construct includes a population of stromal cells (e.g., fibroblasts) in an amount of from about 7×10⁵ to about 1.8×10¹² stromal cells (e.g., fibroblasts). In some embodiments, the engineered tissue construct includes a population of stromal cells (e.g., fibroblasts) in an amount of from about 8×10⁵ to about 1.8×10¹² stromal cells (e.g., fibroblasts). In some embodiments, the engineered tissue construct includes a population of stromal cells (e.g., fibroblasts) in an amount of from about 9×10⁵ to about 1.8×10¹² stromal cells (e.g., fibroblasts). In some embodiments, the engineered tissue construct includes a population of stromal cells (e.g., fibroblasts) in an amount of from about 1×10⁶ to about 1.8×10¹² stromal cells (e.g., fibroblasts). In some embodiments, the engineered tissue construct includes a population of stromal cells (e.g., fibroblasts) in an amount of from about 2×10⁶ to about 1.8×10¹² stromal cells (e.g., fibroblasts). In some embodiments, the engineered tissue construct includes a population of stromal cells (e.g., fibroblasts) in an amount of from about 3×10⁶ to about 1.8×10¹² stromal cells (e.g., fibroblasts). In some embodiments, the engineered tissue construct includes a population of stromal cells (e.g., fibroblasts) in an amount of from about 4×10⁶ to about 1.8×10¹² stromal cells (e.g., fibroblasts). In some embodiments, the engineered tissue construct includes a population of stromal cells (e.g., fibroblasts) in an amount of from about 5×10⁶ to about 1.8×10¹² stromal cells (e.g., fibroblasts). In some embodiments, the engineered tissue construct includes a population of stromal cells (e.g., fibroblasts) in an amount of from about 6×10⁶ to about 1.8×10¹² stromal cells (e.g., fibroblasts). In some embodiments, the engineered tissue construct includes a population of stromal cells (e.g., fibroblasts) in an amount of from about 7×10⁶ to about 1.8×10¹² stromal cells (e.g., fibroblasts). In some embodiments, the engineered tissue construct includes a population of stromal cells (e.g., fibroblasts) in an amount of from about 8×10⁶ to about 1.8×10¹² stromal cells (e.g., fibroblasts). In some embodiments, the engineered tissue construct includes a population of stromal cells (e.g., fibroblasts) in an amount of from about 9×10⁶ to about 1.8×10¹² stromal cells (e.g., fibroblasts). In some embodiments, the engineered tissue construct includes a population of stromal cells (e.g., fibroblasts) in an amount of from about 1×10⁷ to about 1.8×10¹² stromal cells (e.g., fibroblasts). In some embodiments, the engineered tissue construct includes a population of stromal cells (e.g., fibroblasts) in an amount of from about 2×10⁷ to about 1.8×10¹² stromal cells (e.g., fibroblasts). In some embodiments, the engineered tissue construct includes a population of stromal cells (e.g., fibroblasts) in an amount of from about 3×10⁷ to about 1.8×10¹² stromal cells (e.g., fibroblasts). In some embodiments, the engineered tissue construct includes a population of stromal cells (e.g., fibroblasts) in an amount of from about 4×10⁷ to about 1.8×10¹² stromal cells (e.g., fibroblasts). In some embodiments, the engineered tissue construct includes a population of stromal cells (e.g., fibroblasts) in an amount of from about 5×10⁷ to about 1.8×10¹² stromal cells (e.g., fibroblasts). In some embodiments, the engineered tissue construct includes a population of stromal cells (e.g., fibroblasts) in an amount of from about 6×10⁷ to about 1.8×10¹² stromal cells (e.g., fibroblasts). In some embodiments, the engineered tissue construct includes a population of stromal cells (e.g., fibroblasts) in an amount of from about 7×10⁷ to about 1.8×10¹² stromal cells (e.g., fibroblasts). In some embodiments, the engineered tissue construct includes a population of stromal cells (e.g., fibroblasts) in an amount of from about 8×10⁷ to about 1.8×10¹² stromal cells (e.g., fibroblasts). In some embodiments, the engineered tissue construct includes a population of stromal cells (e.g., fibroblasts) in an amount of from about 9×10⁷ to about 1.8×10¹² stromal cells (e.g., fibroblasts). In some embodiments, the engineered tissue construct includes a population of stromal cells (e.g., fibroblasts) in an amount of from about 1×10⁸ to about 1.8×10¹² stromal cells (e.g., fibroblasts). In some embodiments, the engineered tissue construct includes a population of stromal cells (e.g., fibroblasts) in an amount of from about 2×10⁸ to about 1.8×10¹² stromal cells (e.g., fibroblasts). In some embodiments, the engineered tissue construct includes a population of stromal cells (e.g., fibroblasts) in an amount of from about 3×10⁸ to about 1.8×10¹² stromal cells (e.g., fibroblasts). In some embodiments, the engineered tissue construct includes a population of stromal cells (e.g., fibroblasts) in an amount of from about 4×10⁸ to about 1.8×10¹² stromal cells (e.g., fibroblasts). In some embodiments, the engineered tissue construct includes a population of stromal cells (e.g., fibroblasts) in an amount of from about 5×10⁸ to about 1.8×10¹² stromal cells (e.g., fibroblasts). In some embodiments, the engineered tissue construct includes a population of stromal cells (e.g., fibroblasts) in an amount of from about 6×10⁸ to about 1.8×10¹² stromal cells (e.g., fibroblasts). In some embodiments, the engineered tissue construct includes a population of stromal cells (e.g., fibroblasts) in an amount of from about 7×10⁸ to about 1.8×10¹² stromal cells (e.g., fibroblasts). In some embodiments, the engineered tissue construct includes a population of stromal cells (e.g., fibroblasts) in an amount of from about 8×10⁸ to about 1.8×10¹² stromal cells (e.g., fibroblasts). In some embodiments, the engineered tissue construct includes a population of stromal cells (e.g., fibroblasts) in an amount of from about 9×10⁸ to about 1.8×10¹² stromal cells (e.g., fibroblasts). In some embodiments, the engineered tissue construct includes a population of stromal cells (e.g., fibroblasts) in an amount of from about 1×10⁹ to about 1.8×10¹² stromal cells (e.g., fibroblasts). In some embodiments, the engineered tissue construct includes a population of stromal cells (e.g., fibroblasts) in an amount of from about 2×10⁹ to about 1.8×10¹² stromal cells (e.g., fibroblasts). In some embodiments, the engineered tissue construct includes a population of stromal cells (e.g., fibroblasts) in an amount of from about 3×10⁹ to about 1.8×10¹² stromal cells (e.g., fibroblasts). In some embodiments, the engineered tissue construct includes a population of stromal cells (e.g., fibroblasts) in an amount of from about 4×10⁹ to about 1.8×10¹² stromal cells (e.g., fibroblasts). In some embodiments, the engineered tissue construct includes a population of stromal cells (e.g., fibroblasts) in an amount of from about 5×10⁹ to about 1.8×10¹² stromal cells (e.g., fibroblasts). In some embodiments, the engineered tissue construct includes a population of stromal cells (e.g., fibroblasts) in an amount of from about 6×10⁹ to about 1.8×10¹² stromal cells (e.g., fibroblasts). In some embodiments, the engineered tissue construct includes a population of stromal cells (e.g., fibroblasts) in an amount of from about 7×10⁹ to about 1.8×10¹² stromal cells (e.g., fibroblasts). In some embodiments, the engineered tissue construct includes a population of stromal cells (e.g., fibroblasts) in an amount of from about 8×10⁹ to about 1.8×10¹² stromal cells (e.g., fibroblasts). In some embodiments, the engineered tissue construct includes a population of stromal cells (e.g., fibroblasts) in an amount of from about 9×10⁹ to about 1.8×10¹² stromal cells (e.g., fibroblasts). In some embodiments, the engineered tissue construct includes a population of stromal cells (e.g., fibroblasts) in an amount of from about 1×10¹⁰ to about 1.8×10¹² stromal cells (e.g., fibroblasts). In some embodiments, the engineered tissue construct includes a population of stromal cells (e.g., fibroblasts) in an amount of from about 2×10¹⁰ to about 1.8×10¹² stromal cells (e.g., fibroblasts). In some embodiments, the engineered tissue construct includes a population of stromal cells (e.g., fibroblasts) in an amount of from about 3×10¹⁰ to about 1.8×10¹² stromal cells (e.g., fibroblasts). In some embodiments, the engineered tissue construct includes a population of stromal cells (e.g., fibroblasts) in an amount of from about 4×10¹⁰ to about 1.8×10¹² stromal cells (e.g., fibroblasts). In some embodiments, the engineered tissue construct includes a population of stromal cells (e.g., fibroblasts) in an amount of from about 5×10¹⁰ to about 1.8×10¹² stromal cells (e.g., fibroblasts). In some embodiments, the engineered tissue construct includes a population of stromal cells (e.g., fibroblasts) in an amount of from about 6×10¹⁰ to about 1.8×10¹² stromal cells (e.g., fibroblasts). In some embodiments, the engineered tissue construct includes a population of stromal cells (e.g., fibroblasts) in an amount of from about 7×10¹⁰ to about 1.8×10¹² stromal cells (e.g., fibroblasts). In some embodiments, the engineered tissue construct includes a population of stromal cells (e.g., fibroblasts) in an amount of from about 8×10¹⁰ to about 1.8×10¹² stromal cells (e.g., fibroblasts). In some embodiments, the engineered tissue construct includes a population of stromal cells (e.g., fibroblasts) in an amount of from about 9×10¹⁰ to about 1.8×10¹² stromal cells (e.g., fibroblasts). In some embodiments, the engineered tissue construct includes a population of stromal cells (e.g., fibroblasts) in an amount of from about 1×10¹¹ to about 1.8×10¹² stromal cells (e.g., fibroblasts). In some embodiments, the engineered tissue construct includes a population of stromal cells (e.g., fibroblasts) in an amount of from about 2×10¹¹ to about 1.8×10¹² stromal cells (e.g., fibroblasts). In some embodiments, the engineered tissue construct includes a population of stromal cells (e.g., fibroblasts) in an amount of from about 3×10¹¹ to about 1.8×10¹² stromal cells (e.g., fibroblasts). In some embodiments, the engineered tissue construct includes a population of stromal cells (e.g., fibroblasts) in an amount of from about 4×10¹¹ to about 1.8×10¹² stromal cells (e.g., fibroblasts). In some embodiments, the engineered tissue construct includes a population of stromal cells (e.g., fibroblasts) in an amount of from about 5×10¹¹ to about 1.8×10¹² stromal cells (e.g., fibroblasts). In some embodiments, the engineered tissue construct includes a population of stromal cells (e.g., fibroblasts) in an amount of from about 6×10¹¹ to about 1.8×10¹² stromal cells (e.g., fibroblasts). In some embodiments, the engineered tissue construct includes a population of stromal cells (e.g., fibroblasts) in an amount of from about 7×10¹¹ to about 1.8×10¹² stromal cells (e.g., fibroblasts). In some embodiments, the engineered tissue construct includes a population of stromal cells (e.g., fibroblasts) in an amount of from about 8×10¹¹ to about 1.8×10¹² stromal cells (e.g., fibroblasts). In some embodiments, the engineered tissue construct includes a population of stromal cells (e.g., fibroblasts) in an amount of from about 9×10¹¹ to about 1.8×10¹² stromal cells (e.g., fibroblasts). In some embodiments, the engineered tissue construct includes a population of stromal cells (e.g., fibroblasts) in an amount of from about 1×10¹² to about 1.8×10¹² stromal cells (e.g., fibroblasts).

In some embodiments, the engineered tissue construct includes 0 stromal cells (e.g., fibroblasts). In some embodiments, the engineered tissue construct includes a population of stromal cells (e.g., fibroblasts) in an amount of about 1 stromal cell (e.g., fibroblast). In some embodiments, the engineered tissue construct includes a population of stromal cells (e.g., fibroblasts) in an amount of about 10 stromal cells (e.g., fibroblasts). In some embodiments, the engineered tissue construct includes a population of stromal cells (e.g., fibroblasts) in an amount of about 100 stromal cells (e.g., fibroblasts). In some embodiments, the engineered tissue construct includes a population of stromal cells (e.g., fibroblasts) in an amount of about 1×10³ stromal cells (e.g., fibroblasts). In some embodiments, the engineered tissue construct includes a population of stromal cells (e.g., fibroblasts) in an amount about 2×10³ stromal cells (e.g., fibroblasts). In some embodiments, the engineered tissue construct includes a population of stromal cells (e.g., fibroblasts) in an amount of about 3×10³ stromal cells (e.g., fibroblasts). In some embodiments, the engineered tissue construct includes a population of stromal cells (e.g., fibroblasts) in an amount of about 4×10³ stromal cells (e.g., fibroblasts). In some embodiments, the engineered tissue construct includes a population of stromal cells (e.g., fibroblasts) in an amount of about 5×10³ stromal cells (e.g., fibroblasts). In some embodiments, the engineered tissue construct includes a population of stromal cells (e.g., fibroblasts) in an amount of about 6×10³ stromal cells (e.g., fibroblasts). In some embodiments, the engineered tissue construct includes a population of stromal cells (e.g., fibroblasts) in an amount of about 7×10³ stromal cells (e.g., fibroblasts). In some embodiments, the engineered tissue construct includes a population of stromal cells (e.g., fibroblasts) in an amount of about 8×10³ stromal cells (e.g., fibroblasts). In some embodiments, the engineered tissue construct includes a population of stromal cells (e.g., fibroblasts) in an amount of about 9×10³ stromal cells (e.g., fibroblasts). In some embodiments, the engineered tissue construct includes a population of stromal cells (e.g., fibroblasts) in an amount of about 1×10⁴ stromal cells (e.g., fibroblasts). In some embodiments, the engineered tissue construct includes a population of stromal cells (e.g., fibroblasts) in an amount of about 2×10⁴ stromal cells (e.g., fibroblasts). In some embodiments, the engineered tissue construct includes a population of stromal cells (e.g., fibroblasts) in an amount of about 3×10⁴ stromal cells (e.g., fibroblasts). In some embodiments, the engineered tissue construct includes a population of stromal cells (e.g., fibroblasts) in an amount of about 4×10⁴ stromal cells (e.g., fibroblasts). In some embodiments, the engineered tissue construct includes a population of stromal cells (e.g., fibroblasts) in an amount of about 5×10⁴ stromal cells (e.g., fibroblasts). In some embodiments, the engineered tissue construct includes a population of stromal cells (e.g., fibroblasts) in an amount of about 6×10⁴ stromal cells (e.g., fibroblasts). In some embodiments, the engineered tissue construct includes a population of stromal cells (e.g., fibroblasts) in an amount of about 7×10⁴ stromal cells (e.g., fibroblasts). In some embodiments, the engineered tissue construct includes a population of stromal cells (e.g., fibroblasts) in an amount of about 8×10⁴ stromal cells (e.g., fibroblasts). In some embodiments, the engineered tissue construct includes a population of stromal cells (e.g., fibroblasts) in an amount of about 9×10⁴ stromal cells (e.g., fibroblasts). In some embodiments, the engineered tissue construct includes a population of stromal cells (e.g., fibroblasts) in an amount of about 1×10⁵ stromal cells (e.g., fibroblasts). In some embodiments, the engineered tissue construct includes a population of stromal cells (e.g., fibroblasts) in an amount of about 2×10⁵ stromal cells (e.g., fibroblasts). In some embodiments, the engineered tissue construct includes a population of stromal cells (e.g., fibroblasts) in an amount of about 3×10⁵ stromal cells (e.g., fibroblasts). In some embodiments, the engineered tissue construct includes a population of stromal cells (e.g., fibroblasts) in an amount of about 4×10⁵ stromal cells (e.g., fibroblasts). In some embodiments, the engineered tissue construct includes a population of stromal cells (e.g., fibroblasts) in an amount of about 5×10⁵ stromal cells (e.g., fibroblasts). In some embodiments, the engineered tissue construct includes a population of stromal cells (e.g., fibroblasts) in an amount of about 6×10⁵ stromal cells (e.g., fibroblasts). In some embodiments, the engineered tissue construct includes a population of stromal cells (e.g., fibroblasts) in an amount of about 7×10⁵ stromal cells (e.g., fibroblasts). In some embodiments, the engineered tissue construct includes a population of stromal cells (e.g., fibroblasts) in an amount of about 8×10⁵ stromal cells (e.g., fibroblasts). In some embodiments, the engineered tissue construct includes a population of stromal cells (e.g., fibroblasts) in an amount of about 9×10⁵ stromal cells (e.g., fibroblasts). In some embodiments, the engineered tissue construct includes a population of stromal cells (e.g., fibroblasts) in an amount of about 1×10⁶ stromal cells (e.g., fibroblasts). In some embodiments, the engineered tissue construct includes a population of stromal cells (e.g., fibroblasts) in an amount of about 2×10⁶ stromal cells (e.g., fibroblasts). In some embodiments, the engineered tissue construct includes a population of stromal cells (e.g., fibroblasts) in an amount of about 3×10⁶ stromal cells (e.g., fibroblasts). In some embodiments, the engineered tissue construct includes a population of stromal cells (e.g., fibroblasts) in an amount of about 4×10⁶ stromal cells (e.g., fibroblasts). In some embodiments, the engineered tissue construct includes a population of stromal cells (e.g., fibroblasts) in an amount of about 5×10⁶ stromal cells (e.g., fibroblasts). In some embodiments, the engineered tissue construct includes a population of stromal cells (e.g., fibroblasts) in an amount of about 6×10⁶ stromal cells (e.g., fibroblasts). In some embodiments, the engineered tissue construct includes a population of stromal cells (e.g., fibroblasts) in an amount of about 7×10⁶ stromal cells (e.g., fibroblasts). In some embodiments, the engineered tissue construct includes a population of stromal cells (e.g., fibroblasts) in an amount of about 8×10⁶ stromal cells (e.g., fibroblasts). In some embodiments, the engineered tissue construct includes a population of stromal cells (e.g., fibroblasts) in an amount of about 9×10⁶ stromal cells (e.g., fibroblasts). In some embodiments, the engineered tissue construct includes a population of stromal cells (e.g., fibroblasts) in an amount of about 1×10⁷ stromal cells (e.g., fibroblasts). In some embodiments, the engineered tissue construct includes a population of stromal cells (e.g., fibroblasts) in an amount of about 2×10⁷ stromal cells (e.g., fibroblasts). In some embodiments, the engineered tissue construct includes a population of stromal cells (e.g., fibroblasts) in an amount of about 3×10⁷ stromal cells (e.g., fibroblasts). In some embodiments, the engineered tissue construct includes a population of stromal cells (e.g., fibroblasts) in an amount of about 4×10⁷ stromal cells (e.g., fibroblasts). In some embodiments, the engineered tissue construct includes a population of stromal cells (e.g., fibroblasts) in an amount of about 5×10⁷ stromal cells (e.g., fibroblasts). In some embodiments, the engineered tissue construct includes a population of stromal cells (e.g., fibroblasts) in an amount of about 6×10⁷ stromal cells (e.g., fibroblasts). In some embodiments, the engineered tissue construct includes a population of stromal cells (e.g., fibroblasts) in an amount of about 7×10⁷ stromal cells (e.g., fibroblasts). In some embodiments, the engineered tissue construct includes a population of stromal cells (e.g., fibroblasts) in an amount of about 8×10⁷ stromal cells (e.g., fibroblasts). In some embodiments, the engineered tissue construct includes a population of stromal cells (e.g., fibroblasts) in an amount of about 9×10⁷ stromal cells (e.g., fibroblasts). In some embodiments, the engineered tissue construct includes a population of stromal cells (e.g., fibroblasts) in an amount of about 1×10⁸ stromal cells (e.g., fibroblasts). In some embodiments, the engineered tissue construct includes a population of stromal cells (e.g., fibroblasts) in an amount of about 2×10⁸ stromal cells (e.g., fibroblasts). In some embodiments, the engineered tissue construct includes a population of stromal cells (e.g., fibroblasts) in an amount of about 3×10⁸ stromal cells (e.g., fibroblasts). In some embodiments, the engineered tissue construct includes a population of stromal cells (e.g., fibroblasts) in an amount of about 4×10⁸ stromal cells (e.g., fibroblasts). In some embodiments, the engineered tissue construct includes a population of stromal cells (e.g., fibroblasts) in an amount of about 5×10⁸ stromal cells (e.g., fibroblasts). In some embodiments, the engineered tissue construct includes a population of stromal cells (e.g., fibroblasts) in an amount of about 6×10⁸ stromal cells (e.g., fibroblasts). In some embodiments, the engineered tissue construct includes a population of stromal cells (e.g., fibroblasts) in an amount of about 7×10⁸ stromal cells (e.g., fibroblasts). In some embodiments, the engineered tissue construct includes a population of stromal cells (e.g., fibroblasts) in an amount of about 8×10⁸ stromal cells (e.g., fibroblasts). In some embodiments, the engineered tissue construct includes a population of stromal cells (e.g., fibroblasts) in an amount of about 9×10⁸ stromal cells (e.g., fibroblasts). In some embodiments, the engineered tissue construct includes a population of stromal cells (e.g., fibroblasts) in an amount of about 1×10⁹ stromal cells (e.g., fibroblasts). In some embodiments, the engineered tissue construct includes a population of stromal cells (e.g., fibroblasts) in an amount of about 2×10⁹ stromal cells (e.g., fibroblasts). In some embodiments, the engineered tissue construct includes a population of stromal cells (e.g., fibroblasts) in an amount of about 3×10⁹ stromal cells (e.g., fibroblasts). In some embodiments, the engineered tissue construct includes a population of stromal cells (e.g., fibroblasts) in an amount of about 4×10⁹ stromal cells (e.g., fibroblasts). In some embodiments, the engineered tissue construct includes a population of stromal cells (e.g., fibroblasts) in an amount of about 5×10⁹ stromal cells (e.g., fibroblasts). In some embodiments, the engineered tissue construct includes a population of stromal cells (e.g., fibroblasts) in an amount of about 6×10⁹ stromal cells (e.g., fibroblasts). In some embodiments, the engineered tissue construct includes a population of stromal cells (e.g., fibroblasts) in an amount of about 7×10⁹ stromal cells (e.g., fibroblasts). In some embodiments, the engineered tissue construct includes a population of stromal cells (e.g., fibroblasts) in an amount of about 8×10⁹ stromal cells (e.g., fibroblasts). In some embodiments, the engineered tissue construct includes a population of stromal cells (e.g., fibroblasts) in an amount of about 9×10⁹ stromal cells (e.g., fibroblasts). In some embodiments, the engineered tissue construct includes a population of stromal cells (e.g., fibroblasts) in an amount of about 1×10¹⁰ stromal cells (e.g., fibroblasts). In some embodiments, the engineered tissue construct includes a population of stromal cells (e.g., fibroblasts) in an amount of about 2×10¹⁰ stromal cells (e.g., fibroblasts). In some embodiments, the engineered tissue construct includes a population of stromal cells (e.g., fibroblasts) in an amount of about 3×10¹⁰ stromal cells (e.g., fibroblasts). In some embodiments, the engineered tissue construct includes a population of stromal cells (e.g., fibroblasts) in an amount of about 4×10¹⁰ stromal cells (e.g., fibroblasts). In some embodiments, the engineered tissue construct includes a population of stromal cells (e.g., fibroblasts) in an amount of about 5×10¹⁰ stromal cells (e.g., fibroblasts). In some embodiments, the engineered tissue construct includes a population of stromal cells (e.g., fibroblasts) in an amount of about 6×10¹⁰ stromal cells (e.g., fibroblasts). In some embodiments, the engineered tissue construct includes a population of stromal cells (e.g., fibroblasts) in an amount of about 7×10¹⁰ stromal cells (e.g., fibroblasts). In some embodiments, the engineered tissue construct includes a population of stromal cells (e.g., fibroblasts) in an amount of about 8×10¹⁰ stromal cells (e.g., fibroblasts). In some embodiments, the engineered tissue construct includes a population of stromal cells (e.g., fibroblasts) in an amount of about 9×10¹⁰ stromal cells (e.g., fibroblasts). In some embodiments, the engineered tissue construct includes a population of stromal cells (e.g., fibroblasts) in an amount of about 1×10¹¹ stromal cells (e.g., fibroblasts). In some embodiments, the engineered tissue construct includes a population of stromal cells (e.g., fibroblasts) in an amount of about 2×10¹¹ stromal cells (e.g., fibroblasts). In some embodiments, the engineered tissue construct includes a population of stromal cells (e.g., fibroblasts) in an amount of about 3×10¹¹ stromal cells (e.g., fibroblasts). In some embodiments, the engineered tissue construct includes a population of stromal cells (e.g., fibroblasts) in an amount of about 4×10¹¹ stromal cells (e.g., fibroblasts). In some embodiments, the engineered tissue construct includes a population of stromal cells (e.g., fibroblasts) in an amount of about 5×10¹¹ stromal cells (e.g., fibroblasts). In some embodiments, the engineered tissue construct includes a population of stromal cells (e.g., fibroblasts) in an amount of about 6×10¹¹ stromal cells (e.g., fibroblasts). In some embodiments, the engineered tissue construct includes a population of stromal cells (e.g., fibroblasts) in an amount of about 7×10¹¹ stromal cells (e.g., fibroblasts). In some embodiments, the engineered tissue construct includes a population of stromal cells (e.g., fibroblasts) in an amount of about 8×10¹¹ stromal cells (e.g., fibroblasts). In some embodiments, the engineered tissue construct includes a population of stromal cells (e.g., fibroblasts) in an amount of about 9×10¹¹ stromal cells (e.g., fibroblasts). In some embodiments, the engineered tissue construct includes a population of stromal cells (e.g., fibroblasts) in an amount of about 1×10¹² stromal cells (e.g., fibroblasts). In some embodiments, the engineered tissue construct includes a population of stromal cells (e.g., fibroblasts) in an amount of about 1.8×10¹² stromal cells (e.g., fibroblasts).

### Combination of Hepatocytes and Stromal Cells

The cellular compositions disclosed herein can be provided as a suspension containing the hepatocytes and optionally the stromal cells (e.g., fibroblasts).

In some embodiments, the ratio of hepatocytes to stromal cells (e.g., fibroblasts) is between 1:10 and 4:1 (e.g., 1:10 and 4:1, 1:10 and 3:1, 1:10 and 2:1, 1:10 and 1:1, 1:9 and 4:1, 1:9 and 3:1, 1:9 and 2:1, 1:9 and 1:1, 1:8 and 4:1, 1:8 and 3:1, 1:8 and 2:1, 1:8 and 1:1, 1:7 and 4:1, 1:7 and 3:1, 1:7 and 2:1, 1:7 and 1:1, 1:6 and 4:1, 1:6 and 3:1, 1:6 and 2:1, 1:6 and 1:1, 1:5 and 4:1, 1:5 and 3:1, 1:5 and 2:1, 1:5 and 1:1, 1:4 and 4:1, 1:4 and 3:1, 1:4 and 2:1, 1:4 and 1:1, 1:3 and 4:1, 1:3 and 3:1, 1:3 and 2:1, 1:3 and 1:1, 1:2 and 4:1, 1:2 and 3:1, 1:2 and 2:1, 1:2 and 1:1, 1:1 and 4:1, 1:1 and 3:1, 1:1 and 2:1, and 1:0 and 1:1).

For example, in some embodiments, the ratio of hepatocytes to stromal cells (e.g., fibroblasts) is between 1:9 and 4:1. In some embodiments, the ratio of hepatocytes to stromal cells (e.g., fibroblasts) is between 1:8 and 4:1. In some embodiments, the ratio of hepatocytes to stromal cells (e.g., fibroblasts) is between 1:7 and 4:1. In some embodiments, the ratio of hepatocytes to stromal cells (e.g., fibroblasts) is between 1:6 and 4:1. In some embodiments, the ratio of hepatocytes to stromal cells (e.g., fibroblasts) is between 1:5 and 4:1. In some embodiments, the ratio of hepatocytes to stromal cells (e.g., fibroblasts) is between 1:4 and 4:1. In some embodiments, the ratio of hepatocytes to stromal cells (e.g., fibroblasts) is between 1:3 and 4:1. In some embodiments, the ratio of hepatocytes to stromal cells (e.g., fibroblasts) is between 1:2 and 4:1. In some embodiments, the ratio of hepatocytes to stromal cells (e.g., fibroblasts) is between 1:1 and 4:1. In some embodiments, the ratio of hepatocytes to stromal cells (e.g., fibroblasts) is between 1:0 and 4:1.

In some embodiments, the ratio of hepatocytes to stromal cells (e.g., fibroblasts) is between 1:10 and 3:1. In some embodiments, the ratio of hepatocytes to stromal cells (e.g., fibroblasts) is between 1:9 and 3:1. In some embodiments, the ratio of hepatocytes to stromal cells (e.g., fibroblasts) is between 1:8 and 3:1. In some embodiments, the ratio of hepatocytes to stromal cells (e.g., fibroblasts) is between 1:7 and 3:1. In some embodiments, the ratio of hepatocytes to stromal cells (e.g., fibroblasts) is between 1:6 and 3:1. In some embodiments, the ratio of hepatocytes to stromal cells (e.g., fibroblasts) is between 1:5 and 3:1. In some embodiments, the ratio of hepatocytes to stromal cells (e.g., fibroblasts) is between 1:4 and 3:1. In some embodiments, the ratio of hepatocytes to stromal cells (e.g., fibroblasts) is between 1:3 and 3:1. In some embodiments, the ratio of hepatocytes to stromal cells (e.g., fibroblasts) is between 1:2 and 3:1. In some embodiments, the ratio of hepatocytes to stromal cells (e.g., fibroblasts) is between 1:1 and 3:1. In some embodiments, the ratio of hepatocytes to stromal cells (e.g., fibroblasts) is between 1:0 and 3:1.

In some embodiments, the ratio of hepatocytes to stromal cells (e.g., fibroblasts) is between 1:10 and 2:1. In some embodiments, the ratio of hepatocytes to stromal cells (e.g., fibroblasts) is between 1:9 and 2:1. In some embodiments, the ratio of hepatocytes to stromal cells (e.g., fibroblasts) is between 1:8 and 2:1. In some embodiments, the ratio of hepatocytes to stromal cells (e.g., fibroblasts) is between 1:7 and 2:1. In some embodiments, the ratio of hepatocytes to stromal cells (e.g., fibroblasts) is between 1:6 and 2:1. In some embodiments, the ratio of hepatocytes to stromal cells (e.g., fibroblasts) is between 1:5 and 2:1. In some embodiments, the ratio of hepatocytes to stromal cells (e.g., fibroblasts) is between 1:4 and 2:1. In some embodiments, the ratio of hepatocytes to stromal cells (e.g., fibroblasts) is between 1:3 and 2:1. In some embodiments, the ratio of hepatocytes to stromal cells (e.g., fibroblasts) is between 1:2 and 2:1. In some embodiments, the ratio of hepatocytes to stromal cells (e.g., fibroblasts) is between 1:1 and 2:1. In some embodiments, the ratio of hepatocytes to stromal cells (e.g., fibroblasts) is between 1:0 and 2:1.

In some embodiments, the ratio of hepatocytes to stromal cells (e.g., fibroblasts) is between 1:10 and 1:1. In some embodiments, the ratio of hepatocytes to stromal cells (e.g., fibroblasts) is between 1:9 and 1:1. In some embodiments, the ratio of hepatocytes to stromal cells (e.g., fibroblasts) is between 1:8 and 1:1. In some embodiments, the ratio of hepatocytes to stromal cells (e.g., fibroblasts) is between 1:7 and 1:1. In some embodiments, the ratio of hepatocytes to stromal cells (e.g., fibroblasts) is between 1:6 and 1:1. In some embodiments, the ratio of hepatocytes to stromal cells (e.g., fibroblasts) is between 1:5 and 1:1. In some embodiments, the ratio of hepatocytes to stromal cells (e.g., fibroblasts) is between 1:4 and 1:1. In some embodiments, the ratio of hepatocytes to stromal cells (e.g., fibroblasts) is between 1:3 and 1:1. In some embodiments, the ratio of hepatocytes to stromal cells (e.g., fibroblasts) is between 1:2 and 1:1. In some embodiments, the ratio of hepatocytes to stromal cells (e.g., fibroblasts) is between 1:1 and 1:1. In some embodiments, the ratio of hepatocytes to stromal cells (e.g., fibroblasts) is between 1:0 and 1:1.

In some embodiments, the ratio of hepatocytes to stromal cells (e.g., fibroblasts) is between 1:10 and 1:0. In some embodiments, the ratio of hepatocytes to stromal cells (e.g., fibroblasts) is between 1:9 and 1:0. In some embodiments, the ratio of hepatocytes to stromal cells (e.g., fibroblasts) is between 1:8 and 1:0. In some embodiments, the ratio of hepatocytes to stromal cells (e.g., fibroblasts) is between 1:7 and 1:0. In some embodiments, the ratio of hepatocytes to stromal cells (e.g., fibroblasts) is between 1:6 and 1:0. In some embodiments, the ratio of hepatocytes to stromal cells (e.g., fibroblasts) is between 1:5 and 1:0. In some embodiments, the ratio of hepatocytes to stromal cells (e.g., fibroblasts) is between 1:4 and 1:0. In some embodiments, the ratio of hepatocytes to stromal cells (e.g., fibroblasts) is between 1:3 and 1:0. In some embodiments, the ratio of hepatocytes to stromal cells (e.g., fibroblasts) is between 1:2 and 1:0. In some embodiments, the ratio of hepatocytes to stromal cells (e.g., fibroblasts) is between 1:1 and 1:0.

### Biocompatible Hydrogel Scaffolds

The engineered tissue constructs may include a biocompatible scaffold or matrix. The biocompatible scaffold may be liquid, gel, semi-solid, or solid at room temperature (e.g., 25° C.). The biocompatible scaffold may be biodegradable or non-biodegradable. In some embodiments, the scaffold is bioresorbable or bioreplaceable. Exemplary biocompatible scaffolds include polymers and hydrogels including collagen, fibrin, chitosan, MATRIGEL™, dextrans including chemically cross-linkable or photo-cross-linkable dextrans, processed tissue matrix such as submucosal tissue, PEG hydrogels (e.g., heparin-conjugated PEG hydrogels), poly(lactic-co-glycolic acid) (PLGA), hydroxyethyl methacrylate (HEMA), gelatin, alginate, agarose, polysaccharides, hyaluronic acid (HA), peptide-based self-assembling gels, thermo-responsive poly(NIPAAm). A number of biopolymers are known to those skilled in the art (Bryant and Anseth, J. Biomed. Mater. Res. (2002) 59(1):63-72; Mann et al., Biomaterials (2001) 22 (22): 3045-3051; Mann et al., Biomaterials (2001) 22 (5):439-444, and Peppas et al., Eur. J. Pharm. Biopharm. (2000) 50(1), 27-46; all incorporated by reference). In other embodiments, the biocompatible scaffold may contain a biopolymer having any of a number of growth factors, adhesion molecules, degradation sites or bioactive agents to enhance cell viability or for any of a number of other reasons. Such molecules are well known to those skilled in the art.

In some embodiments, the PEG hydrogel may be chemically cross-linkable and/or modified with bifunctional groups.

In certain embodiments, biocompatible scaffold includes allogeneic components, autologous components, or both allogeneic components and autologous components. In certain embodiments, the biocompatible scaffold includes synthetic or semi-synthetic materials. In certain embodiments, the biocompatible scaffold includes a framework or support, such as a fibrin-derived scaffold.

Biocompatible hydrogel scaffolds suitable for use include any polymer that is gellable in situ, e.g., one that does not require chemicals or conditions (e.g., temperature or pH) that are not cytocompatible. This includes both stable and biodegradable biopolymers.

Polymers for use herein are preferably crosslinked, for example, ionically crosslinked. In certain embodiments, the methods and constructs described herein use polymers in which polymerization can be promoted photochemically (i.e., photo crosslinked), by exposure to an appropriate wavelength of light (i.e., photopolymerizable) or a polymer which is weakened or rendered soluble by light exposure or other stimulus. Although some of the polymers listed above are not inherently light sensitive (e.g., collagen, HA), they may be made light sensitive by the addition of acrylate or other photosensitive groups.

In certain embodiments, the method utilizes a photoinitiator. A photoinitiator is a molecule that is capable of promoting polymerization of hydrogels upon exposure to an appropriate wavelength of light as defined by the reactive groups on the molecule. In the context of the disclosure, photoinitiators are cytocompatible. A number of photoinitiators are known that can be used with different wavelengths of light. For example, 2,2-dimethoxy-2-phenyl-acetophenone, HPK 1-hydroxycyclohexyl-phenyl ketone and Irgacure 2959 (hydroxyl-1-[4-(hydroxyethoxy)phenyl]-2methyl-1 propanone) are all activated with UV light (365 nm). Other crosslinking agents activated by wavelengths of light that are cytocompatible (e.g., blue light) can also be used with the methods described herein.

In other embodiments, the method involves the use of polymers bearing non-photochemically polymerizable moieties. In certain embodiments, the non-photochemically polymerizable moieties are Michael acceptors. Non-limiting examples of such Michael acceptor moieties include α,β-unsaturated ketones, esters, amides, sulfones, sulfoxides, phosphonates. Additional non-limiting examples of Michael acceptors include quinines and vinyl pyridines. In some embodiments, the polymerization of Michael acceptors is promoted by a nucleophile. Suitable nucleophiles include, but are not limited to thiols, amines, alcohols and molecules possessing thiol, amine and alcohol moieties. In certain embodiments, the disclosure features use of thermally crosslinked polymers.

In some embodiments, the biocompatible scaffold includes a synthetic heparin mimetic. In particular, the synthetic polymer of the invention may include an amount of negative charge that, in some embodiments, is similar to the amount of negative charge present in heparin. Accordingly, the synthetic polymer of the disclosure can mimic the functional properties of heparin. For example, the synthetic polymer of the disclosure has the potential to bind various bioactive agents, e.g., growth factors, that naturally bind to heparin. Therefore, the synthetic polymer of the disclosure, as well as the hydrogel comprising the synthetic polymer described herein can bind various bioactive agents, e.g., growth factors, thereby preventing the bioactive agents from diffusing away and maintaining the bioactive agents at a high concentration locally, so that they can act on cells and promote various cell functions.

### Methods for Producing Engineered Tissue Constructs

The manufacturing involves two cell types: hepatocytes (e.g., primary human hepatocytes (PHH)) and optionally stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts) human vas deferens fibroblasts). Frozen master cell banks (MCB) were sourced through external suppliers and were received as cryopreserved cells. All cell types are terminally differentiated cells isolated from primary donors obtained with appropriate donor consent for therapeutic use. For example, hepatocytes (e.g., PHH) are obtained from cadaveric donors via collagenase perfusion, Percoll density gradient purification, and subsequent cryopreservation to create a MCB. Hepatocytes are stored cryopreserved until initiation of a manufacturing build. Prior to accepting the lot as a released MCB, release testing is conducted on hepatocyte (e.g., PHH) candidate MCBs to establish that their performance characteristics meet acceptance criteria for characterization, release, and stability. Stromal cells ((e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts) human vas deferens fibroblasts)) are, for example, isolated from a single donor of neonatal foreskin by physical separation of dermal and epidermal layers and sequential digestion with dispase and collagenase. After isolation, stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts) human vas deferens fibroblasts)) are minimally expanded and cryopreserved to create a frozen MCB. Frozen MCBs are shipped to the manufacturing site and stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts) human vas deferens fibroblasts)) are expanded to create working cell banks (WCB), which are then cryopreserved until initiation of a manufacturing build. These WCB are released based on specific acceptance criteria prior to use in the manufacturing process.

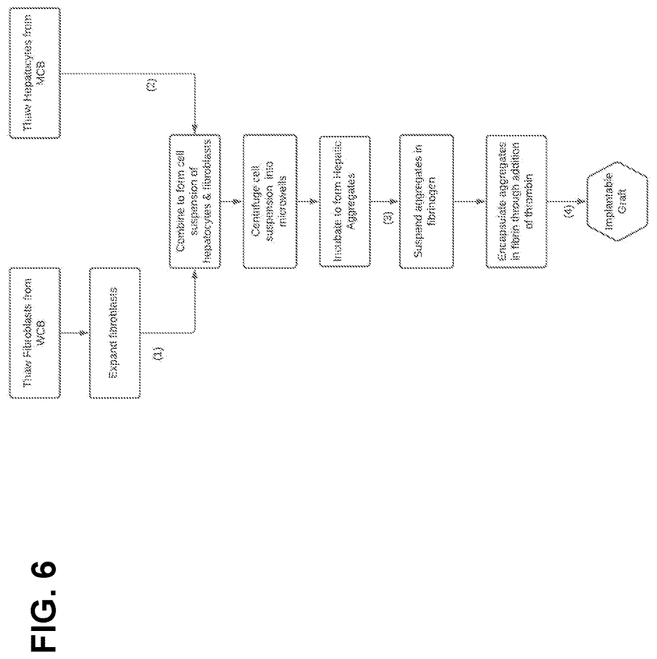

An overview of the continuous manufacturing process for a build is shown in FIG. 6. Upon initiation of a manufacturing build, stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts) human vas deferens fibroblasts)) are thawed from their respective WCB (FIG. 6; Step 1), expanded, and tested to measure viability and cell count. Hepatocytes (e.g., PHH) are thawed from the hepatocyte MCB and tested to measure viability and cell count (FIG. 6; Step 2) prior to optionally being combined at a ratio (e.g., 1:2) with stromal cells (e.g., fibroblasts e.g., human dermal fibroblasts (e.g., normal human dermal fibroblasts, neonatal foreskin fibroblasts, human lung fibroblasts, human ventricular cardiac fibroblasts, human atrial cardiac fibroblasts, human uterine fibroblasts, human bladder fibroblasts, human gingival fibroblasts, human pericardial fibroblasts, human gall bladder fibroblasts, human portal vein fibroblasts) human vas deferens fibroblasts)), centrifuged into arrays of microwells (e.g., pyramidal microwells), and incubated for 2-3 days to promote self-assembly of the cells into multicellular hepatic aggregates. Hepatic aggregates are deemed acceptable for encapsulation after microscopic confirmation of compaction.

The hepatocyte and optional stromal aggregates are then encapsulated with a solution (e.g., a fibrinogen solution) that is polymerized (e.g., with thrombin; FIG. 6; Step 6). These encapsulation steps occur within a mold (e.g., a cylindrical mold) that controls the overall dimensions of the engineered tissue construct to be about 2 mm in thickness and with an outer diameter of 6 mm to 100 cm (e.g., 7 mm to 999 mm, 8 mm to 998 mm, 9 mm to 997 mm, 10 mm to 996 mm, 20 mm to 995 mm, 30 mm to 990 mm, 40 mm to 980 mm, 60 mm to 960 mm, 90 mm to 930 mm, 100 mm to 900 mm, 200 mm to 800 mm, 300 mm to 700 mm, 400 mm to 600 mm, or 500 mm). The thickness is controlled by the volume of cell-hydrogel suspension and targeted to be about 2 mm in thickness.

The engineered tissue constructs of the present disclosure can be formed by a process described herein. In some embodiments, engineered tissue constructs with defined cellular configurations in a biocompatible hydrogel scaffold may be prepared by photopatterning PEG hydrogels containing the hepatocyte and fibroblast cell populations, resulting in a hydrogel network consisting of 3D cell hepatocytes and stromal cells (e.g., fibroblasts). Further control of cell orientation within these patterned domains may be achieved utilizing dielectrophoretic patterning techniques. Dielectrophoresis (DEP) can be used alone for patterning of cells in relatively homogeneous slabs of hydrogel or in conjunction with the photopolymerization method.

In some embodiments, organizing cells and material into spatial arrangements, such as engineered tissue constructs, can be accomplished by physically constraining the placement of cells/material by the use of wells or grooves, or injecting cells into microfluidic channels or oriented void spaces/pores. In certain embodiments, the cells can be organized by physically positioning cells with electric fields, magnetic tweezers, optical tweezers, ultrasound waves, pressure waves, or micromanipulators.

In certain embodiments, the method for fabricating engineered tissue constructs and embedding the constructs in extracellular matrix includes (1) generating 3D templates that have been defined with channels or trenches, (2) suspending the population of cells and the population of cells in liquid collagen and centrifuging these cells into the channels of the template, (3) removing excess cell/collagen suspension to allow aggregates including hepatocytes and stromal cells (e.g., fibroblasts) to form, and (4) removing aggregates from templates via encapsulation in an extracellular matrix scaffold.

In some embodiments, the method for fabricating the engineered tissue constructs includes (1) suspending the population of cells in a naturally-derived and/or synthetic scaffolding, (2) placing the suspended cells into the channels of a 3D template, and (3) allowing the cells to form one or more aggregates at least partially embedded in the naturally-derived and/or synthetic scaffolding. In some embodiments, the 3D template can be generated by molding, templating, photolithography, printing, deposition, sacrificial molding, stereolithography, or a combination thereof.

In some embodiments, an engineered tissue construct can be fabricated through the use a custom 3D printer technology to extrude lattices of carbohydrate glass filaments with predefined diameters, spacings and orientations. For example, in some embodiments, soluble (clinical-grade, sterile) fibrinogen and thrombin are combined and poured over the lattice. After the solution has polymerized into insoluble fibrin, the carbohydrate filaments are dissolved, leaving behind channels within the fibrin. The channels can then be filled with a suspension of cells in a naturally derived or synthetic scaffolding (e.g., soluble type I collagen) that subsequently is polymerized to trap the cells within the channels.

The methods allow for the formation of three-dimensional scaffolds from hundreds of micrometers to tens of centimeters in length and width, and tens of micrometers to hundreds of micrometers in height. A resolution of up to 100 micrometers in the photopolymerization method and possible single cell resolution (10 μm) in the DEP method is achievable. Photopolymerization apparatus, DEP apparatus, and other methods to produce 3-dimensional co-cultures are described in U.S. Pat. No. 8,906,684, which is incorporated herein by reference.

The cells can be cultured in vitro under various culture conditions. The cells can be expanded in culture, e.g., grown under conditions that promote their proliferation. Culture medium can be liquid or semi-solid, e.g., containing agar, methylcellulose, etc. The cell population can be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g., penicillin and streptomycin. The culture can contain growth factors to which the regulatory T cells are responsive. Growth factors, as defined herein, can be molecules capable of promoting survival, growth and/or differentiation of cells, either in culture or in the intact tissue, through specific effects on a transmembrane receptor. Growth factors include polypeptides and non-polypeptide factors.

The cells produced by the methods described herein can be used immediately. Alternatively, the cells can be frozen at liquid nitrogen temperatures and stored for long periods of time, being thawed and capable of being reused. For example, the cells can be frozen in 10% dimethylsulfoxide (DMSO), 50% serum, 40% buffered medium, or some other such solution as is commonly used in the art to preserve cells at such freezing temperatures and thawed in a manner as commonly known in the art for thawing frozen cultured cells.

### Implantation of Engineered Tissue Constructs

The engineered cellular compositions described herein can be implanted in a subject. Non-limiting examples of nonhuman subjects include non-human primates, dogs, cats, mice, rats, guinea pigs, rabbits, fowl, pigs, horses, cows, goats, or sheep. In certain embodiments, the subject can be any animal. In certain embodiments, the subject can be any mammal. In certain embodiments, the subject can be a human.

In some embodiments, the engineered tissue construct is implanted into the subject at an implantation site selected from the group consisting of the peritoneum (e.g., retroperitoneum), peritoneal cavity (e.g., omentum or mesentery), rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization. For example, in some embodiments, the peritoneum is the retroperitoneum. In some embodiments, the peritoneal cavity is the omentum. In some embodiments, the peritoneal cavity is the mesentery. In some embodiments, the omentum is the greater omentum or the omental bursa. In some embodiments, the mesentery is the small intestinal mesentery. In some embodiments, the engineered tissue construct is implanted into the subject as a pedicled omental wrap or an omental wrap.

The engineered tissue construct can be implanted in any suitable manner, often with pharmaceutically acceptable carriers. In some embodiments, the engineered tissue construct is implanted at the site of a tissue or organ. In some embodiments, the engineered tissue construct is implanted at an orthotopic site. In other embodiments, the engineered tissue construct is implanted at an ectopic site.

In some embodiments, the engineered tissue construct is implanted at any site that is suitable for neovascularization. In some embodiments, a site that it suitable for neovascularization includes a site with a microvessel density of from about 3.6 vessels/mm² to about 4500 vessels/mm² (e.g., 3.7 vessels/mm² to about 4000 vessels/mm², 3.8 vessels/mm² to about 3500 vessels/mm², 3.9 vessels/mm² to about 3000 vessels/mm², 4 vessels/mm² to about 2500 vessels/mm², 5 vessels/mm² to about 2000 vessels/mm², 10 vessels/mm² to about 1000 vessels/mm², or about 100 vessels/mm²).

In some embodiments, a site that is suitable for neovascularization may have an existing microvessel density of greater than about 3.7 vessels/mm². In some embodiments, a site that is suitable for neovascularization may have an existing microvessel density of greater than about 3.8 vessels/mm². In some embodiments, a site that is suitable for neovascularization may have an existing microvessel density of greater than about 3.9 vessels/mm². In some embodiments, a site that is suitable for neovascularization may have an existing microvessel density of greater than about 4 vessels/mm². In some embodiments, a site that is suitable for neovascularization may have an existing microvessel density of greater than about 4.1 vessels/mm². In some embodiments, a site that is suitable for neovascularization may have an existing microvessel density of greater than about 4.2 vessels/mm². In some embodiments, a site that is suitable for neovascularization may have an existing microvessel density of greater than about 4.3 vessels/mm². In some embodiments, a site that is suitable for neovascularization may have an existing microvessel density of greater than about 4.4 vessels/mm². In some embodiments, a site that is suitable for neovascularization may have an existing microvessel density of greater than about 4.5 vessels/mm². In some embodiments, a site that is suitable for neovascularization may have an existing microvessel density of greater than about 5 vessels/mm². In some embodiments, a site that is suitable for neovascularization may have an existing microvessel density of greater than about 6 vessels/mm². In some embodiments, a site that is suitable for neovascularization may have an existing microvessel density of greater than about 7 vessels/mm². In some embodiments, a site that is suitable for neovascularization may have an existing microvessel density of greater than about 8 vessels/mm². In some embodiments, a site that is suitable for neovascularization may have an existing microvessel density of greater than about 9 vessels/mm². In some embodiments, a site that is suitable for neovascularization may have an existing microvessel density of greater than about 10 vessels/mm². In some embodiments, a site that is suitable for neovascularization may have an existing microvessel density of greater than about 50 vessels/mm². In some embodiments, a site that is suitable for neovascularization may have an existing microvessel density of greater than about 100 vessels/mm². In some embodiments, a site that is suitable for neovascularization may have an existing microvessel density of greater than about 200 vessels/mm². In some embodiments, a site that is suitable for neovascularization may have an existing microvessel density of greater than about 300 vessels/mm². In some embodiments, a site that is suitable for neovascularization may have an existing microvessel density of greater than about 400 vessels/mm². In some embodiments, a site that is suitable for neovascularization may have an existing microvessel density of greater than about 500 vessels/mm². In some embodiments, a site that is suitable for neovascularization may have an existing microvessel density of greater than about 600 vessels/mm². In some embodiments, a site that is suitable for neovascularization may have an existing microvessel density of greater than about 700 vessels/mm². In some embodiments, a site that is suitable for neovascularization may have an existing microvessel density of greater than about 800 vessels/mm². In some embodiments, a site that is suitable for neovascularization may have an existing microvessel density of greater than about 900 vessels/mm². In some embodiments, a site that is suitable for neovascularization may have an existing microvessel density of greater than about 1000 vessels/mm². In some embodiments, a site that is suitable for neovascularization may have an existing microvessel density of greater than about 2000 vessels/mm². In some embodiments, a site that is suitable for neovascularization may have an existing microvessel density of greater than about 3000 vessels/mm². In some embodiments, a site that is suitable for neovascularization may have an existing microvessel density of greater than about 4000 vessels/mm². In some embodiments, a site that is suitable for neovascularization may have an existing microvessel density of greater than about 4100 vessels/mm². In some embodiments, a site that is suitable for neovascularization may have an existing microvessel density of greater than about 4200 vessels/mm². In some embodiments, a site that is suitable for neovascularization may have an existing microvessel density of greater than about 4300 vessels/mm². In some embodiments, a site that is suitable for neovascularization may have an existing microvessel density of greater than about 4400 vessels/mm². In some embodiments, a site that is suitable for neovascularization may have an existing microvessel density of greater than about 4500 vessels/mm². Autologous, allogenic or xenogenic cells may be used. The cells may be implanted in any physiologically acceptable medium. In one embodiment, the cells are cryopreserved in 5-20% DMSO, 5% dextrose and autologous serum. As is familiar to those of skill in the art, dosage of the cells of the present invention to be implanted in vivo is determined with reference to various parameters, including the species of the host, the age, weight, and disease status. Dosage also depends upon the location to be targeted within the subject. For example, implantation of the engineered tissue construct into the omentum may require different dosages than implantation to the mesentery. The dosage is preferably chosen so that implantation causes an effective result, which can be measured by molecular assays (e.g., a liver function test) or by monitoring a suitable symptom in the subject (e.g., symptoms of hyperammonemia).

In some embodiments, the method further includes administering an immunosuppressive or immunomodulatory drug to modulate an immune response. In some embodiments, the immune response is a humoral response or antibody-mediated response.

In some embodiments, the implantation method prevents graft rejection or promotes graft survival.

The engineered tissue constructs disclosed herein can be administered in combination with one or more additional immunosuppressive therapies including; but not limited to drugs which inhibit T-cell activation (e.g.; calcineurin inhibitors (CNI)); systemic immunosuppressants for universal transplant immunotolerance (corticosteroids such as methylprednisolone (MEDROL® or; SOLU-MEDROL®); prednisone or prednisolone); CNI such as tacrolimus (PROGRAF® or; ASTAFRAF®); cyclosporine (NEORAL®; SANDIMMUNE® or; GENGRAF®); co-stimulation blockade therapy such as Abatacept (ORENCIA®) and Belatacept (NULOJIX®); anti-metabolites such as Mycophenolate motefil (CELLCEPT® or; MYFORTIC®); Azathioprine (IMURAN®); mTORI such as Sirolimus (RAPAMUNE®); Everolimus (AFINITOR®); T-cell depleting monoclonal antibodies such as muromonab-CD3 (OKT3); Alemtuzumab (Campath® or; LEMTRADA®); ATG (THYMOBLOBULIN® or; ATGAM®); B-cell depleting monoclonal antibodies such as rituximab (RITUXAN®); proteasome inhibitors such as Bortezomib (VELCADE®); IL-2-Ra monoclonal antibodies such as daclizumab (ZENAPAX®); Basiliximab (SIMULECT®); lymphocyte integrin blockade monoclonal antibodies such as Natalizumab (TYSABRI®); N-Acetyl Cysteine (NAC); hepatitis B vaccine (HEPLISAV-B®); glecaprevir and pibrentasvir (MAVYRET®); sofosbuvir (VOSEVI®); obeticholic acid (OCALIVA®); elbasvir and grazoprevir (ZEPATIER®); cholic acid (CHOLBAM®); daclatasvir (DAKLINZA®); ombitasvir, paritaprevir and ritonavir (TECHNIVIE™); simeprevir (OLYSIO™); sofosbuvir (SOVALDI®); telaprevir (INCIVEK™); boceprevir (VICTRELIS™); tenofovir disoproxil fumarate (VIREAD®); telbivudine (TYZEKA™); entecavir (BARACLUDE™); adefovir (HEPSERA®); peginterferon alfa-2a (PEGASYS®); peginterferon alfa-2b (PEGINTRON®); or ribavirin and twinrix. Additional agents include gliltazones and vitamin E.

In some embodiments, the engineered tissue constructs disclosed herein can be administered in combination with one or more additional immunosuppressive therapies including but not limited to a PEGylated anti-CD28 monovalent monoclonal antibody fragment (e.g., anti-human CD28 FR104) or domain antibody such as lulizumab (BMS-931699), an IL-2Ra specific antibody for T_{reg} expansion (e.g., a Fc IL-2 mutein (e.g., AMG-592)), a PEGylated IL-2 antibody, a humanized IgG1 anti-CD40L antagonist (e.g., AT-1501), a bivalent anti-CD40L domain antibody such as letolizumab (BMS-986004), an Fc silent human IgG1 anti-CD40 antibody such as VIB4920 or iscalimab (CFZ533), imlifidase, or a human anti-IL6 monoclonal antibody such as clazakizumab (CSL300).

### Recommended Clinical Parameters for Monitoring Following Implantation of the Engineered Tissue Construct

Following implantation of the engineered tissue construct, the subject exhibits a change in one or more clinical parameters. For example, in some embodiments, following implantation of the engineered tissue construct, the subject exhibits a level of serum ammonia in the age-adjusted norm of less than or equal to about 80 μmol/L (e.g., less than about 79 μmol/L, 78 μmol/L, 77 μmol/L, 76 μmol/L, 75 μmol/L, 74 μmol/L, 73 μmol/L, 72 μmol/L, 71 μmol/L, 70 μmol/L, 69 μmol/L, 68 μmol/L, 67 μmol/L, 66 μmol/L, 65 μmol/L, 64 μmol/L, 63 μmol/L, 62 μmol/L, 61 μmol/L, 60 μmol/L, 50 μmol/L, 40 μmol/L, 30 μmol/L, 20 μmol/L, 25 μmol/L, or 10 μmol/L). Alternatively, for example, in some embodiments, following implantation of the engineered tissue construct, the subject exhibits a level of serum ammonia of less than or equal to about 500 μmol/L (e.g., less than about 499 μmol/L, 488 μmol/L, 487 μmol/L, 486 μmol/L, 485 μmol/L, 480 μmol/L, 470 μmol/L, 460 μmol/L, 450 μmol/L, 400 μmol/L, 300 μmol/L, 200 μmol/L, 100 μmol/L, 50 μmol/L, 40 μmol/L, 30 μmol/L, 20 μmol/L, or 10 μmol/L).

In some embodiments, following implantation of the engineered tissue construct, the subject exhibits an improvement in a test of gallbladder ejection fraction (e.g., a hepatobiliary iminodiacetic acid scan). As yet another example, in some embodiments, following implantation of the engineered tissue construct, the subject exhibits a change in one or more parameters in a blood test relative to a reference level.

### Blood Test

In some embodiments, following implantation of the engineered tissue construct, the subject exhibits a change in one or more parameters in a blood test (e.g., a liver function test (LFT), an ammonia test, or a bilirubin test). In some embodiments, the subject exhibits a change in one or more parameters (e.g., albumin, gamma-glutamyl transferase (GGT) level, alkaline phosphatase (ASP) level, aspartate aminotransferase (AST) level, alanine aminotransferase (ALT level), or bilirubin level) in a blood test relative to a reference level following implantation of the engineered tissue construct.

### Albumin

In some embodiments, following implantation of the engineered tissue construct, the subject exhibits a change in the level of albumin, which can be measured with a LFT. For example, in some embodiments, following implantation of the engineered tissue construct, the subject exhibits a change in the level of albumin, such that their albumin level is returned to the age-adjusted norm.

For example, if the subject was a human toddler (e.g., 6-12 months old), it would be determined that a subject exhibits a albumin level that is returned to the age-adjusted norm when the subject's albumin level is within the normal range of about 30-55 U/L (e.g., 31-55 U/L, 32-55 U/L, 33-55 U/L, 34-55 U/L, 35-55 U/L, 36-55 U/L, 37-55 U/L, 38-55 U/L, 39-55 U/L, 40-55 U/L, 41-55 U/L, 42-55 U/L, 43-55 U/L, 44-55 U/L, 45-55 U/L, 46-55 U/L, 47-55 U/L, 48-55 U/L, 49-55 U/L, 50-55 U/L, 51-55 U/L, 52-55 U/L, 53-55 U/L, or 54-55 U/L).

Alternatively, for example, if the subject was a human aged 1-45 years old, it would be determined that a subject exhibits a albumin level that is returned to the age-adjusted norm when the subject's albumin level is within the normal range of about 40-50 U/L (e.g., about 41-50 U/L, 42-50 U/L, 43-50 U/L, 44-50 U/L, 45-50 U/L, 46-50 U/L, 47-50 U/L, 48-50 U/L, or 49-50 U/L).

If the subject was a human aged 46-90 years old, it would be determined that a subject exhibits an albumin level that is returned to the age-adjusted norm when the subject's albumin level is within the normal range of about 35-50 U/L (e.g., about 36-50 U/L, 37-50 U/L, 38-50 U/L, 39-50 U/L, 40-50 U/L, 41-50 U/L, 42-50 U/L, 43-50 U/L, 44-50 U/L, 45-50 U/L, 46-50 U/L, 47-50 U/L, 48-50 U/L, or 49-50 U/L).

### Gamma-Glutamyl Transferase

In some embodiments, following implantation of the engineered tissue construct, the subject exhibits a change in the level of GGT, which can be measured with a LFT. For example, in some embodiments, following implantation of the engineered tissue construct, the subject exhibits a change in the level of GGT, such that their GGT level is returned to the age-adjusted norm.

For example, if the subject was a human toddler (e.g., 6-12 months old), it would be determined that a subject exhibits a GGT level that is returned to the age-adjusted norm when the subject's GGT level is within the normal range of about 1-39 U/L (e.g., 2-39 U/L, 3-39 U/L, 4-39 U/L, 5-39 U/L, 6-39 U/L, 7-39 U/L, 8-39 U/L, 9-39 U/L, 10-39 U/L, 11-39 U/L, 12-39 U/L, 13-39 U/L, 14-39 U/L, 15-39 U/L, 16-39 U/L, 17-39 U/L, 18-39 U/L, 19-39 U/L, 20-39 U/L, 21-39 U/L, 22-39 U/L, 23-39 U/L, 24-39 U/L, 25-39 U/L, 26-39 U/L, 27-39 U/L, 28-39 U/L, 29-39 U/L, 30-39 U/L, 31-39 U/L, 32-39 U/L, 33-39 U/L, 34-39 U/L, 35-39 U/L, 36-39 U/L, 37-39 U/L, or 38-39 U/L).

Alternatively, for example, if the subject was a human child aged 1-5 years old, it would be determined that a subject exhibits a GGT level that is returned to the age-adjusted norm when the subject's GGT level is within the normal range of about 3-22 U/L (e.g., about 3-22 U/L, 4-22 U/L, 5-22 U/L, 6-22 U/L, 7-22 U/L, 8-22 U/L, 9-22 U/L, 10-22 U/L, 11-22 U/L, 12-22 U/L, 13-22 U/L, 14-22 U/L, 15-22 U/L, 16-22 U/L, 17-22 U/L, 18-22 U/L, 19-22 U/L, 20-22 U/L, and 21-22 U/L).

### Alkaline Phosphatase

In some embodiments, following implantation of the engineered tissue construct, the subject exhibits a change in the level of ASP, which can be measured with a LFT. For example, in some embodiments, following implantation of the engineered tissue construct, the subject exhibits a change in the level of ASP, such that their ASP level is returned to the age-adjusted norm.

For example, if the subject was a human, it would be determined that a subject exhibits an ASP level that is returned to the age-adjusted norm when the subject's ASP level is within the normal range of about 50 to 300 U/L e.g., about 51 to 300 U/L, about 52 to U/L, about 53 to 300 U/L, about 54 to 300 U/L, about 55 to 300 U/L, about 56 to 300 U/L, about 57 to 300 U/L, about 58 to 300 U/L, about 59 to 300 U/L, about 60 to 300 U/L, about 65 to 300 U/L, about 70 to 300 U/L, about 80 to 300 U/L, about 90 to 300 U/L, about 100 to 300 U/L, about 125 to 300 U/L, about 150 to 300 U/L, about 175 to 300 U/L, about 200 to 300 U/L, about 225 to 300 U/L, about 250 to 300 U/L, or about 275 to 300 U/L).

### Aspartate Aminotransferase

In some embodiments, following implantation of the engineered tissue construct, the subject exhibits a change in the level of AST, which can be measured with a LFT. For example, in some embodiments, following implantation of the engineered tissue construct, the subject exhibits a change in the level of AST, such that their AST level is returned to the age-adjusted norm.

For example, if the subject was a human, it would be determined that a subject exhibits a AST level that is returned to the age-adjusted norm when the subject's AST level is within the normal range of less than 50 U/L (e.g., less than 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Alanine Aminotransferase

In some embodiments, following implantation of the engineered tissue construct, the subject exhibits a change in the level of ALT, which can be measured with an LFT. For example, in some embodiments, following implantation of the engineered tissue construct, the subject exhibits a change in the level of ALT, such that their ALT level is returned to the age-adjusted norm.

For example, if the subject was a human, it would be determined that a subject exhibits a ALT level that is returned to the age-adjusted norm when the subject's ALT level is within the normal range of less than 50 U/L (e.g., less than 51 U/L, 52 U/L, 53 U/L, 54 U/L, 55 U/L, 56 U/L, 57 U/L, 58 U/L, 59 U/L, 60 U/L, 61 U/L, 62 U/L, 63 U/L, 64 U/L, 65 U/L, 66 U/L, 67 U/L, 68 U/L, 69 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Bilirubin

In some embodiments, following implantation of the engineered tissue construct, the subject exhibits a change in the level of total bilirubin, which can be measured with a bilirubin test. For example, in some embodiments, following implantation of the engineered tissue construct, the subject exhibits a change in the level of total bilirubin, such that their total bilirubin level is returned to the age-adjusted norm of less than about 1.2 mg/dL (e.g., less than about 1.2 mg/dL, 1.1 mg/dL, 1 mg/dL, 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL).

In some embodiments, following implantation of the engineered tissue construct, the subject exhibits a change in the level of direct bilirubin, which can be measured with a bilirubin test. For example, in some embodiments, following implantation of the engineered tissue construct, the subject exhibits a change in the level of direct bilirubin, such that their direct bilirubin level is returned to the age-adjusted norm of less than about 1.7 mg/dL (e.g., less than about 1.6 mg/dL, 1.5 mg/dL, 1.4 mg/dL, 1.3 mg/dL, 1.2 mg/dL, 1.1 mg/dL, 1 mg/dL, 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL).

In some embodiments, following implantation of the engineered tissue construct, the subject exhibits a change in the level of bilirubin, which can be measured with a bilirubin test. For example, in some embodiments, following implantation of the engineered tissue construct, the subject exhibits a change in the level of bilirubin, such that their bilirubin level is returned to the age-adjusted norm of less than about 1 mg/dL (less than about 0.9 mg/dL, 0.8 mg/dL, 0.7 mg/dL, 0.6 mg/dL, 0.5 mg/dL, 0.4 mg/dL, 0.3 mg/dL, 0.2 mg/dL, or 0.1 mg/dL).

### Kits

The compositions described herein can be provided in a kit for use in treating hyperammonemia (e.g., in a subject having a urea cycle disorder, organic acidemia, congenital lactic acidosis, fatty acid oxidation defect, dibasic amino acid deficiencies, transient hyperammonemia, Reye syndrome, severe perinatal asphyxia, virus-associated hyperammonemia, infection, drug-induced hyperammonemia, liver disease, acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder, gastric bypass surgery, gastrointestinal bleeding, malnutrition, generalized seizures, organ transplantation, and/or renal failure). The kit may include one or more engineered tissue construct as described herein. The kit can include a package insert that instructs a user of the kit, such as a physician, to perform any one of the methods described herein. The kit may optionally include surgical equipment or another device for administering the composition. In some embodiments, the kit may include one or more additional therapeutic agents.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. In Vivo Evaluation of Implanted Engineered Tissue Constructs

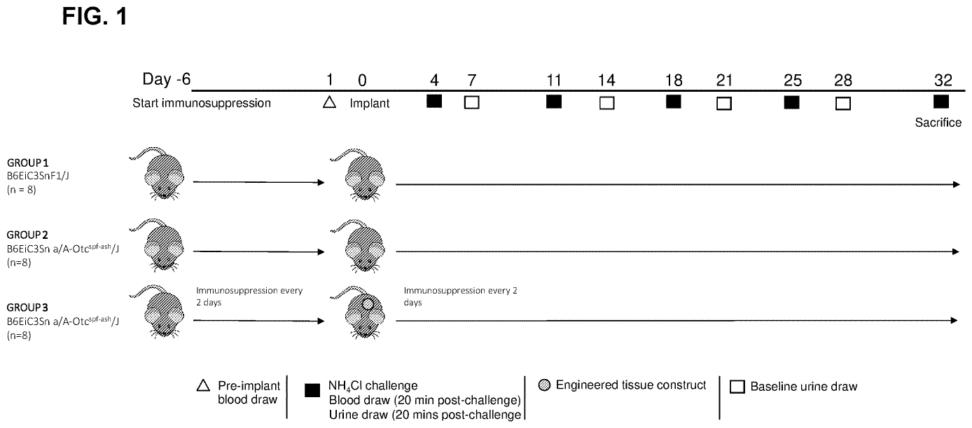

To determine the effectiveness of engineered tissue constructs in reducing ammonia levels a study was performed with three groups of mice. All groups included eight female mice (N=8) of 4-8 weeks of age. Group 1 consisted of control (B6EiC3SnF1/J) mice. Group 2 consisted of hypomorph transgenic mice having the sparse fur-abnormal skin and hair mutation (Otc^{spf-ash}) on the X chromosome. The (Otc^{spf-ash}) mutation results in the reduction of ornithine transcarbamylase (OTC), a critical enzyme in the urea cycle, activity in the liver. The reduction of hepatic OTC activity in this mouse model of chronic hyperammonemia is usually from 5-10% compared to wild-type mice. This decreased OTC activity results in elevated concentration of ammonia in blood plasma. Group 1 (healthy controls) and Group 2 (unhealthy controls) mice were not subjected to surgery. Group 3 consisted of (Otc^{spf-ash}) transgenic mice, as in Group 2, but Group 3 were administered an immunosuppressant composition every two days (FIG. 1). The immunosuppressant composition was administered every two days for six days prior to Day 1 of the study. One day before the start of the study, a blood draw was performed on all three groups to obtain a baseline plasma ammonia concentration measurement. At Day 0, Group 3 was implanted with two engineered tissue constructs, each consisting of 1.41×10⁶ primary human hepatocytes and 2.82×10⁶ normal human dermal fibroblasts. At Day 4, the first of five NH₄Cl challenges was performed. 7.5 mmol/kg of NH₄Cl was administered to all three groups via intraperitoneal route. The day prior to each NH₄Cl challenge, all animals had their bladders emptied, the urine was discarded, and an overnight fast was initiated. Blood samples (50 μl) were collected before each NH₄Cl challenge, 20 minutes after, and 40 minutes after each NH₄Cl challenge. The other four NH₄Cl challenges were performed on Days 11, 18, 25, and 32. On Days 7, 14, 21, and 28 a baseline urine draw was collected. Mice were euthanized on Day 32 (Day 1). Following euthanasia, the whole liver was collected for each animal, half of the liver was fixed for histological analyses, and the other half was snap frozen in liquid nitrogen for evaluation of OTC activity.

### Results

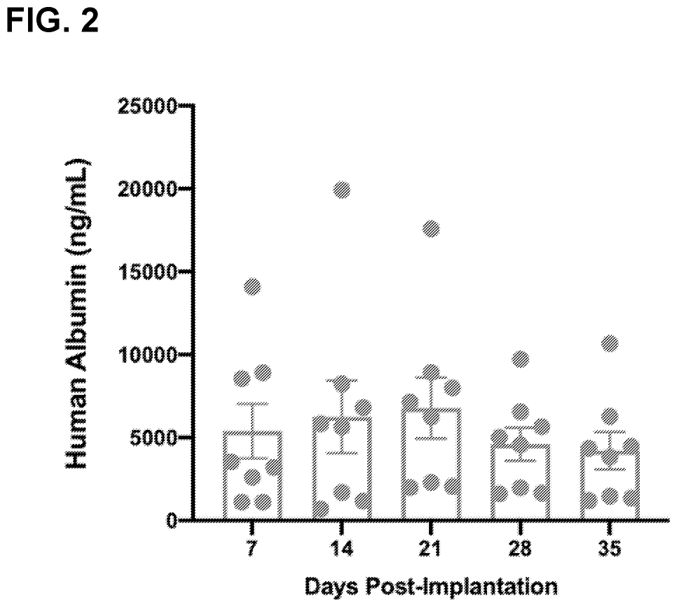

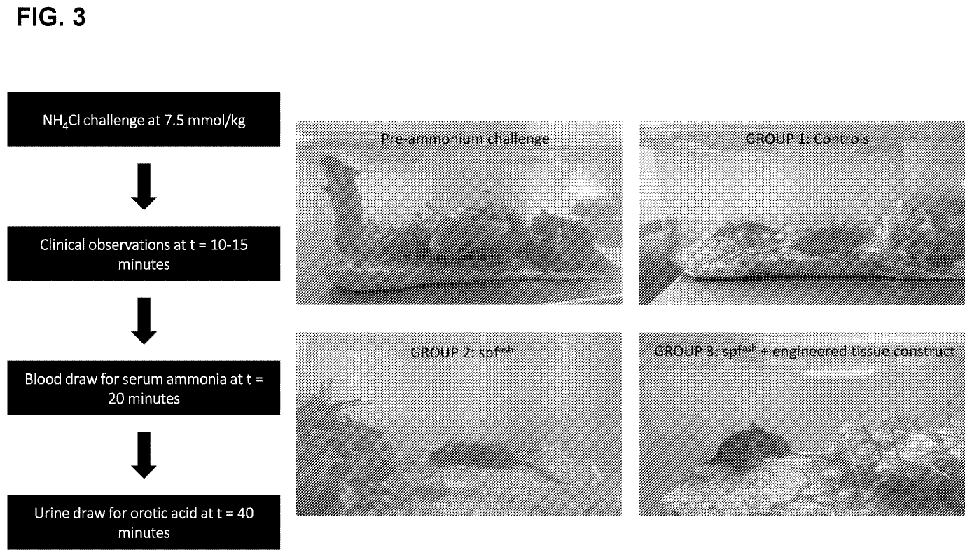

Over time, mice implanted with engineered tissue constructs showed levels of human albumin (ng/mL) (FIG. 2). Clinical observation was performed for all animals starting on Day 1 before ammonia challenge administration, after ammonia challenge administration, once weekly thereafter, and prior to anesthesia (FIG. 3).

Group 3 mice (B6EiC3Sn a/A-Otc^{spf-ash}/J (spf^{ash})) that were implanted with the engineered tissue construct showed significant resiliency to the ammonia challenges compared to Group 1 healthy no-surgery wild-type control (B6EiC3SnF1/J) mice and Group 2 unhealthy no-surgery transgenic B6EiC3Sn a/A-Otc^{spf-ash}/J (spf^{ash}) mice (FIG. 3). Quantitative assessment of behavior for all animals was performed following the behavioral scoring system shown in Table 1. Behavioral scores were assessed for 5 minutes starting 15 minutes after an intraperitonal injection of 7.5 mmol/kg NH₄Cl. The scoring system was based on the appearance of ataxia (A), seizures (S), and abnormal response to sound (R). Ataxia was determined by gently pulling on the mouse's tail and observing gait. A score of 2 was assigned if the mouse was able to ambulate normally; a score of 1 was assigned if the mouse staggered away; a score of 0 signified the mouse was unable to walk. Seizures were categorized as spontaneous myoclonus or tonic-clonic movements. Finally, hyperresponsiveness to sound was determined by ringing a 100-db bell 5-6 times in series and observing mouse behavior.

Based on this scoring system, a normal mouse would be expected to receive a score of seven (A₂S₂R₃). Severely affected mice would receive a score of one (A₀S₁R₀). Any mice that died during the challenge automatically received a score of zero. Mice that exhibited tonic-clonic seizures always died after the seizures. Scoring was performed by two different observers who were blinded to treatment. The inter-rater reliability of this scoring system was examined in preliminary studies by Spearman correlation, correlation=0.95, p<0.0001, n=17 observations.

TABLE 1

| Category | Score | Description of behavior |
|---|---|---|
| Ataxia | 2 | Normal gait |
| | 1 | Abnormal gait |
| | 0 | Unable to support itself; often lying on side |
| Intensity | 2 | No seizures |
| of Seizure | 1 | Myoclonus; spontaneous jerking |
| | 0 | Tonic-clonic seizures; rigid extension of all limbs |
| Response | 3 | No response to sound; normal behavior |
| to sound | 2 | Twitching of extremities |
| | 1 | Jumping |
| | 0 | Moribund; unable to right itself |

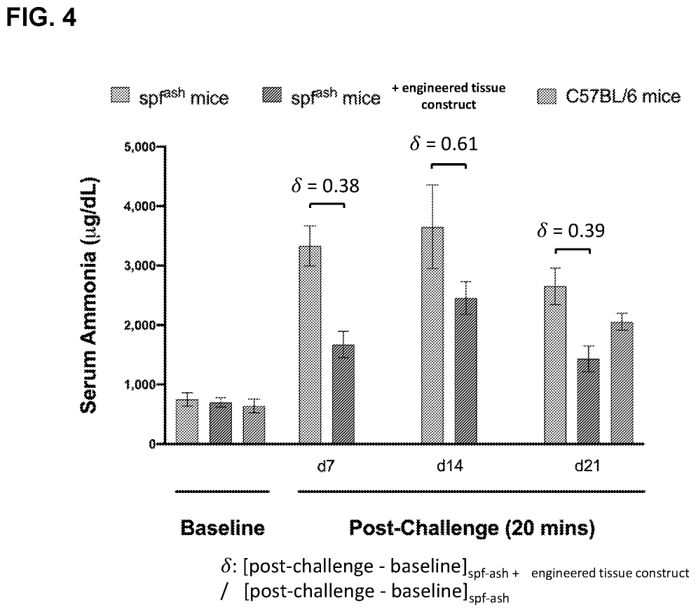

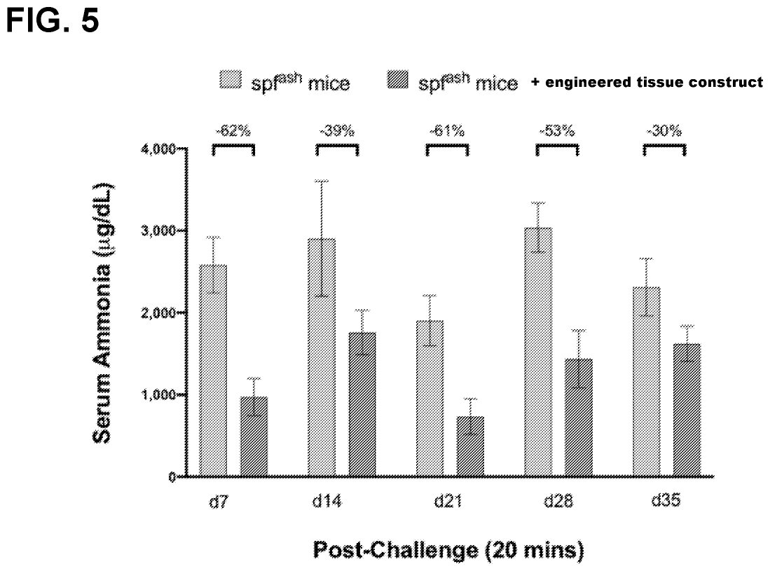

Evaluating the concentration of ammonia in blood serum indicated that Group 3 mice implanted with the engineered tissue construct had significantly lower levels of ammonia 20 minutes after each challenge compared to wild-type Group 1 healthy no-surgery control mice and Group 2 transgenic unhealthy no-surgery control mice (FIG. 4), with the reduction of ammonia persisting up to day 35 (FIG. 5).

### Example 2. Treatment of Hyperammonemia in a Human Patient by Implanting an Engineered Tissue Construct Including Hepatocytes and Stromal Cells

A pediatric patient (age of 1 year old) having hyperammonemia is treated using an engineered tissue construct. After receiving a regimen of immunosuppressants, the patient is administered an engineered tissue construct that includes from about 2×10⁷ to about 6×10¹⁰ hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement gallbladder ejection fraction.

### Example 3. Treatment of Urea Cycle Disorder in a Human Patient by Implanting an Engineered Tissue Construct Including Hepatocytes and Stromal Cells

A pediatric patient (age of 1 year old) having urea cycle disorder is treated using an engineered tissue construct. After receiving a regimen of immunosuppressants, the patient is administered an engineered tissue construct that includes an amount of hepatocytes (e.g., primary human hepatocytes) that is equivalent to 0.5% to 30% of the mass of the liver preserve of the subject and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. The patient shows significant improvement in liver function based on improved blood levels of one or more of gamma-glutamyl transferase, alkaline phosphatase, aspartate aminotransferase, alanine aminotransferase, or albumin.

### Example 4. Treatment of Organic Acidemia in a Human Patient by Implanting an Engineered Tissue Construct Including Hepatocytes and Stromal Cells

A neonate patient (age of 1 day old) having organic acidemia is treated using an engineered tissue construct. After receiving a regimen of immunosuppressants, the patient is administered an engineered tissue construct that includes from about 3×10⁵ to about 3×10¹⁰ (e.g., 1×10⁶ to about 1×10¹⁰, or 1×10⁷ to about 1×10⁹, or about 1×10⁸) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the omental bursa. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 5. Treatment of Congenital Lactic Acidosis in a Human Patient by Implanting an Engineered Tissue Construct Including Hepatocytes and Stromal Cells

A patient (age of 18 years old) having congenital lactic acidosis is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 6. Treatment of Fatty Acid Oxidation Defect in a Human Patient by Implanting an Engineered Tissue Construct Including Hepatocytes and Stromal Cells

A pediatric patient (age of 9 years old) having fatty acid oxidation defect is treated using an engineered tissue construct. After receiving a regimen of immunosuppressants, the patient is administered an engineered tissue construct that includes from about 4.5×10⁷ to about 1.35×10¹¹ (e.g., 5×10⁷ to about 1×10¹¹, 1×10⁸ to about 1×10¹⁰, or about 1×10⁹) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 7. Treatment of Dibasic Amino Acid Deficiencies in a Human Patient by Implanting an Engineered Tissue Construct Including Hepatocytes and Stromal Cells

A patient (age of 60 years old) having dibasic amino acid deficiency is treated using an engineered tissue construct. After receiving a regimen of immunosuppressants, the patient is administered an engineered tissue construct that includes an amount of hepatocytes e.g., primary human hepatocytes) that is equivalent to 0.5% to 30% of the total liver mass of the subject and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 8. Treatment of Transient Hyperammonemia in a Human Patient by Implanting an Engineered Tissue Construct Including Hepatocytes and Stromal Cells

A patient (age of 30 years old) having transient hyperammonemia is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw was performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 9. Treatment of Reye Syndrome in a Human Patient by Implanting an Engineered Tissue Construct Including Hepatocytes and Stromal Cells

A patient (age of 40 years old) having Reye syndrome is treated using an engineered tissue construct. After receiving a regimen of immunosuppressants, the patient is administered an engineered tissue construct that includes an amount of hepatocytes (e.g., primary human hepatocytes) that is equivalent to 0.5% to 20% of the mass of the liver preserve of the subject and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 10. Treatment of Severe Perinatal Asphyxia in a Human Patient by Implanting an Engineered Tissue Construct Including Hepatocytes and Stromal Cells

A pediatric patient (age of 1 year old) having perinatal asphyxia is treated using an engineered tissue construct. After receiving a regimen of immunosuppressants, the patient is administered an engineered tissue construct that includes from about 2×10⁷ to about 6×10¹⁰ (e.g., 1×10⁸ to about 1×10¹⁰, or about 1×10⁹) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 11. Treatment of Virus-Associated Hyperammonemia in a Human Patient by Implanting an Engineered Tissue Construct Including Hepatocytes and Stromal Cells

A patient (age of 55 years old) having virus-associated hyperammonemia is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 12. Treatment of Drug-Induced Hyperammonemia in a Human Patient by Implanting an Engineered Tissue Construct Including Hepatocytes and Stromal Cells

A patient (age of 25 years old) having drug-induced hyperammonemia is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the small intestinal mesentery. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 13. Treatment of Liver Disease in a Human Patient by Implanting an Engineered Tissue Construct Including Hepatocytes and Stromal Cells

A patient (age of 70 years old) having liver disease is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the omental bursa. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 14. Treatment of Acute Liver Failure in a Human Patient by Implanting an Engineered Tissue Construct Including Hepatocytes and Stromal Cells

A patient (age of 45 years old) having acute liver failure is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the omental bursa. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 15. Treatment of Acute-On-Chronic Liver Failure in a Human Patient by Implanting an Engineered Tissue Construct Including Hepatocytes and Stromal Cells

A patient (age of 75 years old) having acute-on-chronic liver failure is treated using an engineered tissue construct. After receiving a regimen of immunosuppressants, the patient is administered an engineered tissue construct that includes an amount of hepatocytes e.g., primary human hepatocytes) that is equivalent to 0.5% to 30% of the total liver mass of the subject and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the omental bursa. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 16. Treatment of Transjugular Intrahepatic Portosystemic Shunt-Induced Hyperammonemia in a Human Patient by Implanting an Engineered Tissue Construct Including Hepatocytes and Stromal Cells

A patient (age of 80 years old) having transjugular intrahepatic portosystemic shunt-induced hyperammonemia is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 17. Treatment of Hepatic Encephalopathy in a Human Patient by Implanting an Engineered Tissue Construct Including Hepatocytes and Stromal Cells

A pediatric patient (age of 9 years old) having hepatic encephalopathy is treated using an engineered tissue construct. After receiving a regimen of immunosuppressants, the patient is administered an engineered tissue construct that includes from about 4.5×10⁷ to about 1.35×10¹¹ (e.g., 5×10⁷ to about 1×10¹¹, 1×10⁸ to about 1×10¹⁰, or about 1×10⁹) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 18. Treatment of Liver Cirrhosis in a Human Patient by Implanting an Engineered Tissue Construct Including Hepatocytes and Stromal Cells

A patient (age of 70 years old) having liver cirrhosis is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### Example 19. Treatment of End-Stage Liver Disease in a Human Patient by Implanting an Engineered Tissue Construct Including Hepatocytes and Stromal Cells

A patient (age of 40 years old) having end-stage liver disease is treated using an engineered tissue construct. After receiving a regimen of immunosuppressants, the patient is administered an engineered tissue construct that includes from about 9×10⁷ to about 1.8×10¹¹ (e.g., 1×10⁸ to about 1×10¹¹ or 1×10⁹ to about 1×10¹⁰) hepatocytes (e.g., 1×10⁸ to about 1×10¹¹ or 1×10⁹ to about 1×10¹⁰) hepatocytes (e.g., primary human hepatocytes) and stromal cells (e.g., fibroblasts) (e.g., human dermal fibroblasts or neonatal foreskin fibroblasts), wherein the ratio of hepatocytes to stromal cells (e.g., fibroblasts) is between 1:10 and 4:1. The engineered tissue construct is implanted in the greater omentum. One month after the implant is introduced, the patient is evaluated. A blood draw is performed and the level of ammonia in the serum is measured to be less than about 50 μmol/L (e.g., less than about 49 μmol/L, 48 μmol/L, 47 μmol/L, 46 μmol/L, 45 μmol/L, 44 μmol/L, 43 μmol/L, 42 μmol/L, 41 μmol/L, 40 μmol/L, 39 μmol/L, 38 μmol/L, 37 μmol/L, 36 μmol/L, 35 μmol/L, 34 μmol/L, 33 μmol/L, 32 μmol/L, 31 μmol/L, 30 μmol/L, 29 μmol/L, 28 μmol/L, 27 μmol/L, 26 μmol/L, 25 μmol/L, 24 μmol/L, 23 μmol/L, 22 μmol/L, 21 μmol/L, 20 μmol/L, 19 μmol/L, 18 μmol/L, 17 μmol/L, 16 μmol/L, 15 μmol/L, 14 μmol/L, 13 μmol/L, 12 μmol/L, 11 μmol/L, or 10 μmol/L). Furthermore, the patient shows significant improvement in liver function based on improved blood levels of one or more of gamma-glutamyl transferase, alkaline phosphatase, aspartate aminotransferase, alanine aminotransferase, or albumin.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating hyperammonemia in a subject having a urea cycle disorder, organic acidemia, congenital lactic acidosis, fatty acid oxidation defect, dibasic amino acid deficiencies, transient hyperammonemia, Reye syndrome, severe perinatal asphyxia, virus-associated hyperammonemia, infection, drug-induced hyperammonemia, liver disease, acute liver failure, acute-on-chronic liver failure, transjugular intrahepatic portosystemic shunt-induced hyperammonemia, hepatic encephalopathy, liver cirrhosis, end-stage liver disease, parenteral hyperalimentation, thyroid disease, Hashimoto encephalopathy, hematologic disorder, gastric bypass surgery, gastrointestinal bleeding, malnutrition, generalized seizures, organ transplantation, or renal failure, the method comprising implanting one or more engineered tissue construct comprising a population of hepatocytes and a population of stromal cells in amounts effective to treat hyperammonemia in the subject.

2. A method of reducing ammonia levels in a subject in need thereof, the method comprising implanting one or more engineered tissue construct comprising a population of hepatocytes and a population of stromal cells in amounts effective to reduce ammonia levels in the subject.

3. The method of claim 1 or 2, wherein the population of hepatocytes comprises an amount of hepatocytes that is equivalent to 0.5% to 30% of the total liver mass of the subject.

4. The method of claim 1 or 2, wherein the population of hepatocytes comprises an amount of hepatocytes that is equivalent to 0.5% to 20% of the mass of the liver reserve of the subject.

5. The method of claim 1 or 2, wherein the population of hepatocytes comprises 3×10⁵ to 1.8×10¹¹ hepatocytes.

6. The method of claim 1 or 2, wherein the population of stromal cells comprises up to 1.8×10¹² stromal cells.

7. The method of any one of claims 1-6, wherein the hepatocytes are primary human hepatocytes.

8. The method of any one of claims 1-7, wherein the stromal cells are fibroblasts.

9. The method of claim 8, wherein the fibroblasts are selected from the group consisting of normal human dermal fibroblasts and neonatal foreskin fibroblasts.

10. The method of claim 9, wherein the fibroblasts are neonatal foreskin fibroblasts.

11. The method of any one of claims 1-10, wherein the ratio of hepatocytes to stromal cells is between 1:10 and 4:1.

12. The method of claim 1 or 2, wherein the engineered tissue construct is from 0.1 mL to 5 L in volume.

13. The method of claim 1 or 2, wherein the density of hepatocytes is 0.1 M/mL to 150 M/mL.

14. The method of any one of claims 1-13, wherein the engineered tissue construct further comprises a biocompatible hydrogel scaffold.

15. The method of claim 14, wherein the biocompatible scaffold comprises fibrin.

16. The method of any one of claims 1-15, wherein the engineered tissue construct is implanted into the subject at an implantation site selected from the group consisting of the peritoneum, peritoneal cavity, rectus abdominis muscle, abdominal oblique muscle, quadriceps femoris muscle, extraperitoneal fat, and renal capsule; an extraperitoneal site, a site on the surface of the liver, or an extrapleural site; or a site that is suitable for neovascularization.

17. The method of claim 16, wherein the peritoneum is the retroperitoneum.

18. The method of claim 16, wherein the peritoneal cavity is the omentum or the mesentery.

19. The method of claim 18, wherein the omentum is the greater omentum or the omental bursa.

20. The method of claim 18, wherein the mesentery is the small intestinal mesentery.

21. The method of claim 16, wherein the implantation site is an extraperitoneal site.

22. The method of claim 16, wherein the implantation site is a site on the surface of the liver.

23. The method of claim 16, wherein the implantation site is an extrapleural site.

24. The method of claim 16, wherein the implantation site is a site that is suitable for neovascularization.

25. The method of any one of claims 1-24, wherein the engineered tissue construct is implanted into the subject at an implantation site that has a microvessel density of greater than about 3.6 vessels/mm².

26. The method of any one of claims 1-25, wherein the subject is a human.

27. The method of any one of claims 1-26, wherein following implantation of the engineered tissue construct, the subject exhibits a level of serum ammonia of less than or equal to about 50 μmol/L.

28. The method of any one of claims 1-27, wherein following implantation of the engineered tissue construct, the subject exhibits a change in one or more parameters in a blood test relative to a reference level.

29. The method of claim 28, wherein the blood test is a liver function test.

30. The method of claim 28 or 29, wherein the one or more parameters comprises the level of gamma-glutamyl transferase, alkaline phosphatase, aspartate aminotransferase, alanine aminotransferase, or albumin.

31. The method of any one of claims 1-30, wherein following implantation of the engineered tissue construct, the subject exhibits an improvement in a test of gallbladder ejection fraction.

32. The method of claim 31, wherein the test is a hepatobiliary iminodiacetic acid scan.

33. The method of claim 1, wherein the infection is a urinary tract infection or an infection caused by Proteus mirabilis, E. coli, or Klebsiella.

34. The method of claim 1, wherein the drug-induced hyperammonemia is caused by the drug valproate acid, topiramate, carbamazepine, salicylate, sulfadiazine, a carbonic anhydrase inhibitor, a carbonic anhydrase inhibitor with valproate acid, or a chemotherapy.

35. The method of claim 1, wherein the liver disease is biliary atresia, alpha-1 antitrypsin deficiency, Wilson disease, cystic fibrosis, galactosemia, or tyrosinemia.

36. The method of claim 1, wherein the hematologic disorder is multiple myeloma or acute leukemia.

37. A kit comprising an engineered tissue construct, wherein the kit further comprises a package insert instructing a user of the kit to implant the engineered tissue construct to the subject in accordance with the method of any one of claims 1-36.
